(19) United States
(12) Patent Application Publication
(10) Pub. No.: US 2021/0292711 A1
(43) Pub. Date: Sep. 23, 2021

(54) METHODS AND COMPOSITIONS FOR ADOPTIVE T CELL THERAPY INCORPORATING INDUCED NOTCH SIGNALING

(71) Applicant: FRED HUTCHINSON CANCER RESEARCH CENTER, Seattle, WA (US)

(72) Inventors: Stanley R. Riddell, Seattle, WA (US); Irwin D. Bernstein, Seattle, WA (US); Margot J. Pont, Seattle, WA (US)

(73) Assignee: FRED HUTCHINSON CANCER RESEARCH CENTER, Seattle, WA (US)

(21) Appl. No.: 17/272,117

(22) PCT Filed: Aug. 28, 2019
(86) PCT No.: PCT/US2019/048574

§ 371 Date: Feb. 26, 2021

Related U.S. Application Data
(60) Provisional application No. 62/723,936, filed on Aug. 28, 2018.

(51) Int. Cl. C12N 5/0783; C07K 14/725; A61K 35/17

(52) U.S. Cl. CPC C12N 5/0636; C12N 2501/42; A61K 35/17; C07K 14/7051

(57) ABSTRACT

The disclosure provides in some aspect methods and compositions related to culturing and engineering T cells that maintain less differentiated state. The T cells are cultured in conditions that induce Notch signaling The resulting T cells exhibit maintenance of a less differentiated state for prolonged periods and have reduced susceptibility to exhaustion. Also provided are the cells produced by the methods, as well as related compositions and methods of use for adoptive therapy.

### CROSS-REFERENCE TO RELATED APPLICATION

This application claims the benefit of U.S. Provisional Application No. 62/723,936, filed Aug. 28, 2018, the entire contents of which are expressly incorporated herein by reference.

### STATEMENT OF GOVERNMENT LICENSE RIGHTS

This invention was made with government support under CA136551 awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 70118_Seq_final_20190827.txt. The text file is 152 KB; was created on Aug. 27, 2019; and is being submitted via EFS-Web with the filing of the specification.

### BACKGROUND

Adoptive T cell therapy is a technique by which T cells are administered to a subject to improve the immune functionality of the subject against a particular target. While the T cells can be sourced from any relevant individual or individuals, many techniques include harvesting initial autologous cells from the subject, expanding the population of T cells ex vivo, and administering the expanded population back to the same subject. An emerging approach is to use allogeneic cells as the starting donor material, which is of critical importance for indications in which the subject's cells are not suitable starting material for genetic engineering or are otherwise fundamentally deficient. Regardless of source, during the ex vivo culture the T cells can be manipulated to further enhance their efficacy. Thus, in addition to mere expansion of cell numbers, the T cells can be selected or modified for certain desired properties (e.g., antigen reactivity or polyfunctionality). For example, the T cells can be genetically modified to express a heterologous gene encoding an immunoreceptor that specifically recognizes an antigen of interest. In CAR T cells, the T cells are genetically modified to express a chimeric antigen receptor (CAR) on the surface. The CAR typically contains an extracellular domain with enhanced affinity for an antigen of interest. The extracellular domain is linked to an intracellular signaling domain that activates the T cell upon antigen binding. Such CAR T cells can provide a powerful tool to combat pathogens and cancer cells because upon binding to the target antigen in vivo, the CAR T cells undergo further expansion and activation to provide a type of “living drug” that can have a direct cytotoxic action against the target as well as influence the endogenous immune functionality through production of cytokines.

While adoptive T cell therapy is theoretically versatile and can be specifically applied to address a variety of conditions, such as cancers, infectious diseases, and autoimmune diseases, there is a growing recognition that achievement of durable clinical responses to the treatment depends on qualities of the transferred T cells that result in proliferation, persistence, and resistance to exhaustion in the tumor microenvironment in the subject after administration. The factors that determine robust T cell expansion ex vivo and in vivo, subsequent persistence in vivo, the durability of the T cell response in vivo, and T cell toxicities in vivo have been challenging to define both in the starting population of T cells that are propagated ex vivo and in the final product that is infused to the patient. This challenge has been due in part to variations in the phenotypic composition of T cells isolated from the source, in particular for patients with malignancies or hyperproliferative disorders of the immune system, such as lymphoma and myeloma.

Accordingly, despite the advances in the art of adoptive T cell therapy, a need remains for methods and compositions that consistently provide robust T cells with enhanced therapeutic efficacy, activity, and persistence in a subject after administration. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of culturing a T cell in vitro. The method comprises exposing a T cell to a medium comprising a Notch receptor agonist for a time sufficient to induce Notch receptor signaling in the cell.

In some embodiments, the T cell is not cultured in the presence of an antigen-presenting cell (APC). In some embodiments, the Notch receptor agonist is not expressed on a cell in the medium. In some embodiments, the T cell is not cultured in the presence of an antigen-presenting cell (APC) expressing a Notch receptor agonist. For example, in some embodiments the Notch receptor agonist is not expressed on an APC in the medium. In some embodiments, the T cell is not cultured in the presence of an antigen-presenting cell (APC) expressing Delta like ligand 4 (DLL4).

In some embodiments, the T cell is a naïve T cell (T_{N}), a memory stem T cell (T_{SCM}), or a central memory T cell (T_{CM}). In one embodiment, the T cell is a naïve T cell (T_{N}). In some embodiments, the T cell is an effector memory T cell (T_{EM}). In some embodiments, the T cell is further characterized as CD62L+. In some embodiments, the T cell is further characterized as CD45RA+. In some embodiments, the T cell is further characterized as CD45RO. In some embodiments, the T cell is further characterized as CD95. In some embodiments, the T cell is further characterized as CCR7+. In some embodiments, the T cell is further characterized as CD62L+, CD45RA+, and CD45RO.

In another aspect, the disclosure provides a method of culturing T cells in vitro, comprising exposing a population of cells to a medium comprising a Notch receptor agonist for a time sufficient to induce Notch receptor signaling in the cells.

In some embodiments, the T cell population is not cultured in the presence of an antigen-presenting cell (APC). In some embodiments, the Notch receptor agonist is not expressed on a cell in the medium. In some embodiments, the T cell population is not cultured in the presence of an antigen-presenting cell (APC) expressing a Notch receptor agonist. For example, in some embodiments, the Notch receptor agonist is not expressed on an APC in the medium. In some embodiments, the T cell population is not cultured in the presence of an antigen-presenting cell (APC) expressing Delta like ligand 4 (DLL4).

In one embodiment, the method is a method of culturing naïve T (T_{N}) cells in vitro, that comprises exposing a population of naïve T (T_{N}) cells to a medium comprising a Notch receptor agonist for a time sufficient to induce Notch receptor signaling in the cell. In some embodiments, the population of T_{N} cells comprises at least about 40%, at least about 50%, at least about 60%, at least about 70%, at least about 80%, at least about 85%, at least about 90%, at least about 95%, at least about 96%, at least about 97%, at least about 98%, at least about 99% or about 100% of T_{N} cells. In some embodiments, the population comprises about 40% to about 90%, about 50% to about 90%, about 60% to about 90%, about 70% to about 90%, about 80% to about 90%, about 40% to about 80%, about 50% to about 80%, about 60% to about 80%, about 70% to about 80%, about 40% to about 70%, about 50% to about 70%, about 60% to about 70%, about 40% to about 60%, about 50% to about 60%, or about 40% to about 50% of T_{N} cells. In some embodiments, the T_{N} cell is further characterized as CD62L+, CD45RA+, CD45RO−, CD95−, and/or CCR7+. In some embodiments, the exposing lasts for a period (“the exposure time”) of at least about 12 hours, at least about one day, at least about two days, at least about three days, at least about four days, at least about five days, at least about six days, at least about a week, at least about eight days, at least about nine days, at least about ten days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about a month. In some embodiments, the exposure time is between one day to 15 days or between two days to 10 days.

In some embodiments, the percentage of the T_{N} cells in the population does not change after the exposing. In some embodiments, the percentage of the T_{N} cells in the population changes, after the exposing, by less than about 1%, less than about 2%, less than about 5%, less than about 10%, less than about 15%, less than about 20%, less than about 25%, less than about 30%, less than about 35%, less than about 40%, less than about 45%, or less than about 50%. In some embodiments, the percentage of the T_{N} cells in the population is: (i) at least about 40% before the exposing and at least about 40% after the exposing; (ii) at least about 50% before the exposing and at least about 40% after the exposing; (iii) at least about 50% before the exposing and at least about 50% after the exposing; (iv) at least about 50% before the exposing and at least about 60% after the exposing; (v) at least about 60% before the exposing and at least about 50% after the exposing; (vi) at least about 60% before the exposing and at least about 60% after the exposing; (vii) at least about 60% before the exposing and at least about 70% after the exposing; (viii) at least about 70% before the exposing and at least about 60% after the exposing; (ix) at least about 70% before the exposing and at least about 70% after the exposing; (x) at least about 70% before the exposing and at least about 80% after the exposing; (xi) at least about 80% before the exposing and at least about 70% after the exposing; (xii) at least about 80% before the exposing and at least about 80% after the exposing; (xiii) at least about 80% before the exposing and at least about 90% after the exposing; (xiv) at least about 90% before the exposing and at least about 80% after the exposing; (xv) at least about 90% before the exposing and at least about 90% after the exposing; or (xvi) at least about 90% before the exposing and about 100% after the exposing. In some embodiments, the T_{N} cell, the population of T_{N} cells, or one or more progeny cells thereof, maintains at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4.0 fold, at least 4.5 fold, at least 5.0 fold, at least 5.5 fold, at least 6.0 fold, at least 6.5 fold, or at least 7.0 fold less-differentiated state in vivo compared to the T_{N} cell that did not receive the Notch receptor agonist.

In some embodiments of the above aspects, the Notch receptor agonist comprises a domain of a mammalian Notch receptor ligand that binds to a mammalian Notch1, Notch2, Notch3, or Notch4 receptor. In some embodiments, the Notch receptor agonist is or comprises a Delta protein, a Jagged protein, an anti-Notch antibody, or a fragment or derivative thereof, that binds to a mammalian Notch receptor, or any combination thereof. In some embodiments, the Notch receptor agonist, upon binding to a Notch receptor, is capable of exposing an S2 cleavage site in the negative regulatory region (NRR) of the Notch receptor. In some embodiments, the Notch receptor agonist, upon binding to a Notch receptor, is capable of exposing an S2 cleavage site in the negative regulatory region (NRR) of the Notch receptor.

In some embodiments, the Notch receptor agonist comprises an extracellular domain of a Delta protein or a Jagged protein. In some embodiments, the Delta protein is or comprises Delta Like Ligand 1 (DLL1), or an extracellular Notch-binding domain thereof. In some embodiments, the Delta protein is or comprises Delta Like Ligand 3 (DLL3, or an extracellular Notch-binding domain thereof. In some embodiments, the Delta protein is or comprises Delta Like Ligand 4 (DLL4), or an extracellular Notch-binding domain thereof. In some embodiments, the Jagged protein is or comprises Jagged1, or an extracellular Notch-binding domain thereof. In some embodiments, the Jagged protein is or comprises Jagged2, or an extracellular Notch-binding domain thereof. In some embodiments, the Notch agonist comprises Dlk1, Dlk2, DNER, EGFL 7, or F3/contactin or a Notch-binding derivative thereof. In some embodiments, the Notch receptor agonist is an anti-Notch antibody, or derivative thereof, that binds to an epitope in the Notch extracellular domain (NECD) that is not in the negative regulatory region (NRR) of the Notch receptor. In some embodiments, the anti-Notch antibody (or antigen binding fragment or derivative thereof) binds to an epitope in the Notch extracellular domain (NECD) that is not in the negative regulatory region (NRR) of Notch1, Notch2, Notch3, and/or Notch4 receptor. In some embodiments, the Notch receptor agonist is immobilized at a concentration of about 0.01 μg/ml to about 100 μg/ml. In some embodiments, a plurality of T cells is exposed to the medium at a concentration sufficient to contact substantially all of the immobilized Notch receptor agonist.

In some embodiments, the medium further comprises one or more cytokines, or a biologically active fragment thereof, that modulate T cell differentiation. In some embodiments, the one or more cytokines include IL-1, IL-lb, IL-2, IL-4, IL-6, IL-7, IL-10, IL-12, IL-15, IL-17, IL-21, IL-23, IL-27, IFN-γ, TNF-α, TGFβ, or any combination thereof, in an effective concentration. In some embodiments, the exposure time of the T cell to the medium is between about 1 and about 15 days. In some embodiments, the exposure time is between about 2 and about 10 days.

In some embodiments, a plurality of T cells is exposed to a medium. In further embodiments, the plurality of T cells is obtained from one or more source subjects before exposing to the medium.

In some embodiments, the method further comprises isolating the T cell, or one or more progeny T cells thereof, from the medium after exposure. In some embodiments wherein a population of T cells (e.g., T_{N} cells) have been exposed, at least about 40%, at least about 50%, at least about 60%, at least about 70%, at least about 80%, at least about 85%, at least about 90%, at least about 95%, or at least about 99% of the population of the T cells (e.g., T_{N} cells) or one or more progeny cells thereof after the exposure are T cells having the characteristics of CD62L+ and CD45RO−.

In some embodiments, the method further comprises administering the T cell (or T cells), or one or more progeny T cells thereof, to a subject in need thereof.

In some embodiments, the method further comprises transducing the cell (or T cells, e.g., T_{N} cells) with a heterologous nucleic acid molecule comprising a sequence that encodes an immune receptor. In some embodiments, the immune receptor is an antigen receptor that comprises an extracellular domain that specifically binds to an antigen of interest, wherein the extracellular domain is operatively linked to an intracellular domain that activates the T cell upon binding of the extracellular domain to the antigen of interest. In some embodiments, the immune receptor is a T cell receptor (TCR) that specifically binds to a peptide of interest bound to a major histocompatibility complex (MHC) molecule.

In another aspect, the disclosure provides an in vitro method of generating a T cell expressing a heterologous immune receptor. The method comprises performing the culturing method described herein; and, during the exposing step, transducing the T cell with a heterologous nucleic acid molecule comprising a sequence that encodes an immune receptor.

In some embodiments, the T cell is a naïve T cell (T_{N}).

In some embodiments, the immune receptor comprises an extracellular domain that specifically binds to an antigen of interest, wherein the extracellular domain is operatively linked to an intracellular domain that activates the T cell upon binding of the extracellular domain to the antigen of interest. In some embodiments, the immune receptor is a T cell receptor (TCR) that specifically binds to a peptide of interest bound to a major histocompatibility complex (WIC) molecule. In some embodiments, the method further comprises administering the T cell, or one or more progeny T cells thereof, to a subject in need thereof.

In another aspect, the disclosure provides a method of adoptive cell therapy. The method comprises administering a therapeutically effective number of cells produced by the in vitro methods of culturing a T cell or T cell population, described herein, to a subject in need thereof. In some embodiments, the cells are produced by in vitro methods of culturing a naïve T cell (T_{N}), or a population thereof, as described herein. In some embodiments, the subject has a condition selected from cancer, infectious disease, and autoimmune disease.

Regarding any methodological aspect described herein, in some embodiments the T cell or population of T cells express a chimeric antigen receptor. In some further embodiments, T cell is a naïve T cell (T_{N}) or the population of T cells comprise T_{N} as described.

In another aspect, the disclosure provides a cell were population cells produced by any one of the methods described herein.

In another aspect, the disclosure provides a therapeutic composition comprising a plurality of cells, as described herein, and an effective carrier.

In another aspect, the disclosure provides a method of reducing or preventing exhaustion of a T_{N} cell expressing a chimeric antigen receptor or the population of T_{N} cells expressing a chimeric antigen receptor. The method comprises exposing the T_{N} cell or the population of T_{N} cells to a medium comprising a Notch receptor agonist for a time sufficient to induce Notch receptor signaling in the cell.

In another aspect, the disclosure provides a method of generating a T_{N} cell expressing a chimeric antigen receptor or a population of T_{N} cells expressing a chimeric antigen receptor. The method comprises modifying the T_{N} cell or the population of T_{N} cells to express a chimeric antigen receptor to a medium comprising a Notch receptor agonist, wherein the Notch receptor agonist reduces or prevents exhaustion of the T_{N} cell.

Regarding any method described herein, in some embodiments the Notch receptor agonist is not expressed on any cell being co-cultured with the T cell(s). For example, in some embodiments, the Notch receptor agonist is not expressed on by APC cells or bone marrow cells (e.g., OP9-DL1 cells). In some embodiments of the methods described herein, the T cell or the population of T cells is not co-cultured with APCs. In some embodiments of the methods described herein, the T cell or the population of T cells is not co-cultured with bone marrow cells. In some embodiments of the methods described herein, the T cell or the population of T cells is not co-cultured with OP9-DL1 cells. In some embodiments of the methods described herein, the T cell is a T_{N} cell, or the population of T cells is a population of T_{N} cells, and the T cell population thereof is not co-cultured with OP9-DL1 cells. In some embodiments of the methods described herein, the T cell is a T_{N} cell, or the population of T cells is a population of T_{N} cells, and the T cell population thereof is not co-cultured with bone marrow cells. In some embodiments of the methods described herein, the Notch receptor agonist is not DLL1.

In another aspect, the disclosure provides a method of culturing a T cell or T cells in vitro, comprising exposing a T cell or a population of T cells, to a medium comprising a Notch receptor agonist for a time sufficient to induce Notch receptor signaling in the cell, wherein the Notch receptor agonist is a peptide ligand, and wherein the T cell is not co-cultured with an OP9-DL1 cell or a bone marrow cell expressing Delta-like 1 (DLL1). In some embodiments, the T cell or the population of T cells exhibits at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 250%, or at least about 300% less exhaustion compared to a T cell co-cultured with OP9-DL1 cells.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 graphically illustrates the relative increase of Notch signaling (as determined by Hes1 expression) of naïve T cells exposed to immobilized DLL1 (a Notch agonist) (μg/ml) over similar cells exposed to immobilized IgG1 (as control ligand, with irrelevant binding), or cells incubated in blank (no ligand) tissue culture (“TC”; as control). Cells were cultured for 4 hours on plates coated with DLL1-^{Ext IgG} with retronectin, IgG1 with retronectin, or TC control plates. Hes1 RNA was isolated, transcribed into cDNA, and quantified using SYBRgreen q-PCR. This culture method results in Hes1 upregulation, showing Notch signaling.

FIGS. 2A and 2B graphically illustrate the relative expression of Notch1 and Notch2 receptors, respectively, in human CD4+ and CD8+ T cells at different time points before and after culture under different conditions. The cultures are indicated in the lower legend (TC=culture plates coated with retronectin only, DLL1=is the Notch agonist for experimental condition, and IgG=culture plates coated with control IgG ligand; all plates coated with retronectin).

FIGS. 3A-3D graphically illustrate the relative expression of Notch ligands (Notch receptor agonists) DLL1, DLL4, JAG1, and JAG2, respectively, in human CD4+ and CD8+ T cells at different time points before and after culture under different conditions. The cultures are indicated in the lower legend (TC=tissue culture, DLL1=is the Notch agonist as the experimental condition, and IgG is the control).

FIG. 4 illustrates gating strategy and cell markers used in exemplary flow cytometry assays to identify differentiated subsets of T cells.

FIGS. 5A-5E graphically illustrate relative proportions of differentiated subsets for human CD4+ or CD8+ T cells, as determined by flow cytometry after incubation with different concentrations Notch ligands DLL1, anti-Notch1 antibody (N1), anti-Notch2 antibody (N2), or DLL4. Prior to analysis, human naïve T cells were stimulated with anti-CD3/CD28 DYNABEADS® (ThermoFisher) and incubated with the indicated Notch agonists, IgG1 (as an irrelevant ligand control), or in simple tissue culture (TC; as a no ligand control) in media containing IL-2. The state differentiation was determined by flow cytometry based on the expression of CD62L and CD45RO. Incubation with each tested Notch ligand resulted in increased proportion of less differentiated T cells (CD62L+ CD45RO− cells, i.e., T_{N}/T_{SCM} subset) for both CD4+ and CD8+ T cells. Specifically, FIGS. 5A and 5B illustrates that incubation with Notch ligand DLL1, at three different concentrations, maintains higher proportion of naïve or less differentiated CD4+ T cells CD8+ T cells, respectively, compared to controls. FIGS. 5C and 5D illustrates that incubation with three different Notch ligands, each at three different concentrations, maintains higher proportion of naïve or less differentiated CD4+ T cells CD8+ T cells, respectively, compared to controls. FIG. 5E illustrates that incubation with Notch ligand DLL4, at three different concentrations, maintains higher proportion of naïve or less differentiated CD8+ T cells, respectively, compared to controls. [

FIGS. 6A and 6B graphically illustrate the total number of T cells measured in cultures initiated with naïve T cells and stimulated with anti-CD3/CD28 DYNABEADS® (ThermoFisher) in the presence of different concentrations of DLL1 ligands, IgG1 control ligand, and in simple tissue culture (no ligand control). Cell numbers are shown at day 7 (FIG. 6A) and day 11 (FIG. 6B) after culture initiation for CD4+ and CD8+ T cells, respectively.

FIG. 7 graphically illustrates the total T cell numbers (CD4+ and CD8+) at day 5 and day 11 for cells cultured in plates pre-coated with 2.5 μg anti-Notch1 antibody (“N1”, an anti-Notch receptor antibody and agonist), IgG1 control ligand, and in simple tissue culture (no ligand control), and stimulated with anti-CD3/CD28 beads. The N1 antibody was adhered to the plastic surface in a manner similar to the DLL1 ligand in the studies described above.

FIG. 8 graphically illustrates tumor burden as measured by bioluminescence imaging (BLI) at times after injection of mice with Raji tumors. Tumor size is indicated for individuals administered with control T cells, tissue culture control (TC) CAR T cells, anti-Notch1 antibody (“N1”) exposed CAR T cells, and IgG1 (an irrelevant ligand control) exposed CAR T cells. Each line represents a mouse, and symbols depict individual data points.

FIGS. 9A-9C graphically illustrate the levels of CAR T cells in the blood at several time points after Raji tumor injection and T cell infusion in NSG mice. Mice were treated with control T cells, tissue culture control (TC) CAR T cells, anti-Notch1 antibody (“N1”) exposed CAR T cells, and IgG1 exposed CAR T cells. Blood was obtained at the indicated time points, lysed with ammonium chloride potassium solution, and then stained with antibodies for CD45, CD4, CD8 and EGFR. Data was collected on a Canto II flow cytometer. FIG. 9A illustrates the frequency of EGFRt+ CD8+ and CD4+ T cells in the total lymphocyte population in the mice. FIG. 9B illustrates the frequency of EGFRt+ CD8+ T cells, and FIG. 9C illustrates the frequency of EGFRt+ CD4+ T cells in the total lymphocyte population in the mice.

FIG. 10A graphically illustrates tumor burden as measured by bioluminescence imaging (BLI) at times after injection of mice with Raji tumors. Tumor size is indicated for individuals administered with control T cells, tissue culture control (TC) CAR T cells, anti-Notch1 antibody (“N1”) exposed CAR T cells, and IgG1 CAR T cells. Each line represents a mouse, and symbols depict individual data points.

FIG. 10B graphically illustrates survival of the mice characterized in FIG. 10A over time after Raji tumor injection. Individuals were administered control T cells, tissue culture (TC) CAR T cells, anti-Notch1 antibody (“N1”) exposed CAR T cells, and IgG1 exposed CAR T cells.

FIGS. 11A-11C graphically illustrate results of repeated stimulation assays to assess the ability of CAR T cells to repetitively kill and eliminate tumor cells. CD8+ T cells were cultured with anti-Notch1 antibody (“N1”, a Notch receptor agonist), IgG, or in mere tissue culture (TC) under comparable conditions and transduced to express a CAR specific for CD19. The cells were then exposed to either K562-CD19 (FIG. 11A) or Raji cells (FIG. 11B) at a 1:1 effector:target ratio in a 96-well plate format. The target cells were not irradiated and the medium lacked IL-2. Additional target cells were added after an additional 48 and 72 hours to provide repeated and constant exposure to tumor antigen. Normalized T cell counts were determined during the assay, indicating that N1-exposed T cells remain active and continue to proliferate (i.e., with minimized exhaustion) even after multiple or constant exposure to the antigen. Flow cytometry of aliquots of the culture showed that the N1 CAR T cells more effectively eliminated tumor cells than IgG1-exposed T cells (FIG. 11C).

FIGS. 12A and 12B graphically illustrate the effect of infusing NSG-Raji mice with different doses of CD4+ CAR T cells cultured with a Notch receptor agonist. Specifically, CD4+ CAR T cells were cultured on plates coated with 2.5 μg of anti-Notch antibody (N1) or IgG1 control. The cells were infused after 11 days of culture. FIG. 12A graphically illustrates the tumor burden, as determined by bioluminescence imaging of firefly luciferase, over time post-infusion of the different doses of CD4+ CAR T cells. FIG. 12B graphically illustrates the percent of CD4+ CAR T cells of lymphocyte singlets in the blood collected at several time points after infusion of the CD4+ CAR T cells. Blood from each collection was lysed with ammonium chloride potassium solution and then stained with antibodies for CD45, CD4, and EGFR. Data was collected on a Canto 2-1 flow cytometer.

FIGS. 13A and 13B graphically illustrate the effect of infusing NSG-Raji mice with different doses of CD8+ CAR T cells cultured with Notch receptor agonist. Specifically, CD8+ CAR T cells were cultured on plates coated with 2.5 μg of anti-Notch antibody (N1) or IgG1 control. The cells were infused after 11 days of culture. FIG. 13A graphically illustrates the tumor burden, as determined by bioluminescence imaging of firefly luciferase, over time post-infusion of the CAR T cells. FIG. 13B graphically illustrates survival of mice after infusion with the CD8+ CAR T cells.

FIGS. 14A-14C graphically illustrate the effect of CD8+ CAR T cells with Notch receptor agonist (anti-Notch antibody, N1) for seven or 11 days prior to infusion into NSG-Raji mice. Specifically, CD8+ CAR T cells were cultured on N1-coated plates or IgG1-coated plates for 7 days or for 7 days followed by 4 days in normal TC flasks (D11 groups). FIG. 14A graphically illustrates the tumor burden, as determined by bioluminescence imaging of firefly luciferase, over time post-infusion of the CD8+ CAR T cells cultured for 7 or 11 days. FIG. 14B graphically illustrates the percent of CAR T cells of lymphocyte singlets in the blood collected at several time points after infusion of the CD8+ CAR T cells cultured for 7 or 11 days. Blood from each collection was lysed with ammonium chloride potassium solution and then stained with antibodies for CD45, CD8, and EGFR. FIG. 14C graphically illustrates survival of mice after infusion with the CD8+ CAR T cells.

FIGS. 15A and 15B graphically illustrate proliferation of CD4+ CAR T cells and CD8+ CAR T cells, respectively, in vivo after infusion with mixtures of CD4+ and CD8+ CAR T cells that were separately incubated with anti-Notch antibody (N1 agonist) or control (IgG antibody). The CD4+ CAR T cells and CD8+ CAR T cells cultured separately and mixed together in the indicated combinations on day 11 at a 1:1 ratio and infused into NSG-Raji mice.

FIGS. 16A-16C illustrate phenotypic characteristics of CD4+ cells that have been cultured in the presence of a Notch receptor agonist (anti-Notch1 antibody N1). FIG. 16A graphically illustrates the mitochondrial membrane potential, as determined by TMRM stain, for CD4+ cells incubated with Notch agonist as compared to cells incubated with IgG antibody control. FIG. 16B graphically illustrates that reduced mitochondrial membrane potential induced by Notch activation occurs mainly in CD45RA high/CD45RO low CD4+ cells. FIG. 16C illustrates the scatterplot of CD8+ cells cultured with Notch receptor agonist (anti-Notch1 antibody N1) or IgG control, demonstrating that Notch-stimulation increases distinct molecular profiles in T cells.

FIG. 17 graphically illustrates the percentage of CD4+ T cells categorized as T_{EM}/T_{EFF}, T_{CM}, or T_{N}/T_{SCM} based on the expression profile of developmental markers. The cells were assayed at days 5, 8, and 11 of culture with N1 Notch agonist or IgG1 control ligand. The cells were initially depleted of T_{N} cells prior to the start of culture.

FIGS. 18A and 18B graphically illustrate the percentage of CD4+ T cells categorized as T_{EM}/T_{EFF}, T_{CM}, or T_{N}/T_{SCM} based on the expression profile of developmental markers. The cells were assayed at days 5, 8, and 11 of culture with N1 Notch agonist or IgG1 control ligand. The cells at the initiation of the culture were sorted CD4+ T_{CM} cells (FIG. 18A) or CD4+ T_{EM} cells (FIG. 18B) at the initiation of the culture.

FIGS. 19A and 19B graphically illustrate the percentage of CD8+ T cells categorized as T_{EM}/T_{EFF}, T_{CM}, or T_{N}/T_{SCM} based on the expression profile of developmental markers. The cells were assayed at days 5, 8, and 11 of culture with N1 Notch agonist or IgG1 control ligand. The cells at the initiation of the culture were sorted CD8+ T_{CM} cells (FIG. 19A) or CD8+ T_{EM} cells (FIG. 19B) at the initiation of the culture.

FIG. 20 graphically illustrates the percentage of bulk T cells categorized as T_{EM}/T_{EFF}, T_{CM}, or T_{N}/T_{SCM} based on the expression profile of developmental markers. The cells were assayed at days 5, 8, and 11 of culture with N1 Notch agonist or IgG1 control ligand. The cells were initially depleted of T_{N} cells prior to the start of culture.

### DETAILED DESCRIPTION

As indicated above, T lymphocyte cells (“T cells) expanded and/or manipulated ex vivo are useful in adoptive cell therapy for a variety of conditions, including cancers, infectious diseases, and autoimmune conditions. Persistence and function of the administered T cells in the subject for a period of time is believed to correlate with the overall robustness and durability of the treatment. However, the factors that determine robust T cell expansion ex vivo and in vivo, persistence in vivo, the durability of the T cell response in vivo, and T cell toxicities in vivo have been challenging to define.

One barrier to a clear understanding of the mechanisms underlying the robustness and durability of adoptive cell therapy is the heterogeneity of T cells isolated from the initial source. Different T cell subsets appear to have unique attributes that differentially impact adoptive cell therapy. For example, in non-human primates it has been demonstrated that adoptive transfer of virus-specific CD8+ T cells derived from central memory T cells (T_{CM}) and not effector memory T cells (T_{EM}) provide long-term functional immunity. Transcriptional and epigenetic profiling of CD8+ T cell subsets suggests progressive differentiation with hierarchical potential for proliferation, persistence, and effector function after adoptive transfer. In mice, serial transfer of single CD8+ _{TCM} cells revealed “stemness” (i.e., self-renewal, differentiation to T_{EM} or effector T cells (T_{E})) and provides protective immunity.

In view of such findings, a progressive model of differentiation of T cells has been proposed, although it is noted that other models of T cell differentiation have also been proposed and are plausible. In the progressive model, circulating T cells that have undergone maturation and selection in the thymus progress through states of differentiation where the proliferative and self-renewal capacities of the cells are inversely proportional to the effector function capacities of the T cells at different states of differentiation. The T cells begin with relatively high proliferative and self-renewal capacities and relatively low effector functions, but gradually transition over the course of differentiation through states that have reduced proliferative and self-renewal capacities but have enhanced effector functions. In this exemplary model, the T cell differentiation states (i.e., identifiable T cell subsets) are designated as transitioning from naïve T cells (T_{N}) to stem central memory T cells (T_{SCM}) to central memory T cells (T_{CM}) to effector memory T cells (T_{EM}) to effector T cells (T_{E} or T_{EFF}). T_{N} cells have the greatest proliferative and self-renewal capacity but the least effector functionality, whereas the T_{E} cells conversely have the least proliferative and self-renewal capacity but the greatest effector functionality. These different subsets can be identified by expression of surface markers. For example, T_{N} are typically considered CD62L+, CD45RA+, CD45RO−, and CD95−; T_{SCM} are typically considered CD62L+, CD45RA+, CD45RO−, and CD95+; T_{CM} are typically considered CD62L+, CD45RA−, CD45RO+, and CD95+; T_{EM} are typically considered CD62L−, CD45RA−, CD45RO+, and CD95+; and T_{E} are typically considered CD62L−, CD45RA+, CD45RO−, and CD95+. Other markers expressed by such differentiated subsets are known.

Approaches have been explored to obtain relatively uniform and undifferentiated T cells. For example, less differentiated T cell subsets (e.g., T_{SCM} and T_{CM}) can be isolated and used to improve anti-tumor efficacy. In another study, culturing T cells in the presence of IL-7, IL-15 and IL-21 retains early memory T cells. Furthermore, Wnt signaling has been shown to arrest effector T cell (T_{E}) differentiation and generates CD8+ memory stem cells (T_{SCM}) Finally, inhibition of Akt signaling has been shown to promote the generation of superior tumor-reactive T cells for adoptive immunotherapy.

It is possible that presence of relatively “young” or undifferentiated T cells can improve long-term efficacy of adoptive cell therapies. While various approaches exist to obtain relatively undifferentiated T cells, they have limitations for practical application in adoptive T cell therapies. For example, one challenge is to maintain the early, undifferentiated phenotype without limiting T cell numbers considering that many strategies, such as augmenting Wnt signaling or inhibiting Akt, negatively affect T cell proliferation.

The role of Notch signaling in the proliferation and differentiation of T cells (including engineered T cells) ex vivo and the implications for practical and long term in vivo therapeutic effect is explored herein.

The Notch signaling pathway is a highly conserved pathway that facilitates cell to cell signaling in metazoan animals. Mammalian Notch receptors (e.g., Notch1, Notch2, Notch3, and Notch4) are Type I transmembrane receptors that are initially expressed in precursor forms with an extracellular domain (NECD), a transmembrane domain, and an intracellular domain (NICD). The precursor is cleaved by a furin convertase to provide the mature receptor with two subunits. One subunit consists of the majority of the NECD, which remains noncovalently associated with the other subunit, which contains the transmembrane domain and NICD. The NECDs of the Notch receptors have a series of epidermal growth factor (EGF) like repeats at the N-terminal side, which play a role in ligand interaction. After the EGF repeats (toward the C-terminal side of the subunit relative to the EGF repeats) are three cysteine rich LIN12 and Notch (LNR) repeats, which play a role in preventing ligand independent signaling, and a heterodimerization domain (HD). The region with the LNR and HD domains is referred to as the negative regulatory region (NRR).

In a typical scenario, Notch signaling is initiated when the NECD binds to an appropriate ligand presented on the surface of an opposing cell. The canonical ligands, ligands Jagged1 (e.g., GenBank Accession No. AAC51731) Jagged2 (e.g., GenBank Accession No. AAD15562), Delta like 1 (DLL1; e.g., GenBank Accession Nos. ABC26875 or NP005609), Delta-like 3 (DLL3; GenBank Accession Nos. NP_982353.1 or NP_058637.1), or Delta-like 4 (DLL4; e.g., GenBank Accession No. NP_061947.1) (the sequence of each accession number incorporated herein by reference), are also Type I transmembrane proteins and have an extracellular domain with an N-terminal region, a cysteine rich Delta-Serrate, and Lag2 (DSL) region, and a varying number of EGF repeats. See, e.g., Falix F., et al., Biochimica et Biophysica Acta (BBA)—Molecular Basis of Disease, 1822(6):988-996 (2012), incorporated herein by reference in its entirety. The binding of an appropriate ligand to the Notch receptor presented from a neighboring cell results in a conformational change that exposes an S2 cleavage site in the NRR of the Notch receptor, permitting proteolysis. The conformational change is thought to result from a mechanical “tug” induced by the internalization by transendocytosis of the ligand into the ligand-expressing cell. Upon the initial cleavage of the Notch receptor at the S2 site, additional proteolysis occurs intracellularly to separate the NICD from the transmembrane domain. The active NICD then translocates to the nucleus and participates in a cascade of transcription activation and suppression pathways. See, e.g., WO 2018/017827, incorporated herein in its entirety, for a discussion of Notch signaling and regulation thereof.

Notch has been shown to play a key role in regulating cell proliferation, differentiation, development, and homeostasis. In adult mammals, Notch signaling continues to play a key role in numerous processes, including neural and hematopoietic stem cell renewal and differentiation, as well as the development of many immune cell subsets. Notch signaling interactions with T cell signaling and stimulation are discussed in Falix F., et al., Biochimica et Biophysica Acta (BBA)—Molecular Basis of Disease, 1822(6):988-996 (2012), incorporated herein by reference in its entirety. Canonical T cell signaling is mediated through the surface T cell receptor (TCR), typically through binding of an appropriate peptide displayed on an MHC presented by a cell, in combination with signaling through a co-stimulatory signal. Notch is also expressed on T cells and can influence T cell differentiation through multiple pathways. In a canonical pathway, Notch signaling promotes expression of Hes1 genes, which in turn promotes TCR signaling by inhibiting the PTEN inhibitor. Furthermore, the endocytosis and digestion of Notch receptors promotes interactions leading to the production of NFκB and promotion of the PI3K/Akt/mTor pathway which can stimulate proliferation, survival, and differentiation.

However, the functional result of Notch signaling is difficult to predict because different cells in different circumstances exhibit different phenotypic effects of Notch signaling. For example, recent studies have suggested that specific interactions mediated by Notch signaling in stem cells contribute to the quiescence of stem cells, which in turn can permit self-renewal and maintenance of pluripotency of the stem cells until they are activated by appropriate stimuli. See, e.g., Dahlberg, et al., Blood 117:6083-6090 (2011), incorporated herein by reference in its entirety, which teaches that Notch2 signaling affects hemopoietic stem cell self-renewal by blocking differentiation into multipotent progenitors (MPP) and myeloid/monocytic (M) cell lineages. Notch1 on the other hand is described as promoting T cell differentiation versus B-cell (B) differentiation from MPPs. The inventors have previously demonstrated that immobilized Delta1^{Ext-IgG} (a Notch ligand) enhances ex vivo expansion of HSC from cord blood by maximizing proliferation while inhibiting differentiation. This effect was observed even in the presence of strong cytokine-driven proliferative signals. Finally, by titration of the immobilized Delta1^{Ext-IgG} agonist, it was shown that Notch signaling in the cell population depends on quantitative signals.

However, Notch signaling has also been shown to stimulate differentiation of MPPs to T cells. As indicated above, Notch signaling has been shown to promote the PI3K/Akt/mTor pathway, which can stimulate proliferation, survival, and differentiation of T cells. Accordingly, the role of Notch signaling in the further differentiation of T cells is controversial, with a variety of transcriptional effects having been observed. Thus, the effects of Notch signaling on proliferation, development, and/or quiescence are likely to be very context-dependent. Ultimately, it is unclear whether Notch will have an inhibitory or activating effect on the differentiation of T cells.

In view of the contradictory effects of Notch signaling during the differentiation of cell types from HSC to mature T_{E} cells, the study disclosed herein was conducted to clarify the role of Notch signaling for potential influence on the differentiation of T cell subtypes, and for potential practical application of this role in cultivating and expanding T cells for adoptive cell therapy.

As described in more detail below, the inventors studied the effect of Notch signaling during ex vivo culture of T cells, the development of CAR T cells, and the influence on the efficacy of such cells after administration into murine tumor model. For example, naïve T cells were exposed to various Notch agonists during expansion and genetic modification introducing a tumor targeting chimeric antigen receptor to determine if Notch signaling would maintain T cells with a phenotype consistent with an earlier (i.e., less) differentiated state, and enhance therapeutic activity. T cells were analyzed for cell surface phenotype after expansion, which suggested a larger fraction of the T cells exposed to Notch signaling retained an early phenotype characterized by greater expression of CD45RA and CD62L. In a further exemplary assay, T cells transduced to express a tumor-specific CAR and cultured with Notch activating agonists exhibited greater expansion, superior long-term persistence, and greater antitumor activity after adoptive transfer into NSG mice engrafted with a human B cell tumor compared to CAR T cells cultured under identical conditions but without Notch activating agonists. Additionally, the Notch-activated cells surprisingly maintained activity and continued to proliferate even after multiple or constant exposure to the antigen, thus demonstrating the ability to avoid exhaustion. While these assays used a model incorporating CD19-specific CAR T cells in combination with engrafted human CD19+ B cell tumors, the observed results are not a function of the particular antigen and corresponding CAR and, thus, would apply to any T cell using adaptive cell therapy, including with any antigen/CAR combination. Further assays addressing CD4+ T cells and CD8+ T cells independently indicated that induced Notch signaling had different effects on these cell populations, and mixing of CD4+ T cells and CD8+ T cells provided synergistic effects related to proliferation and antitumor effect. Accordingly, augmenting Notch signaling during generation of therapeutic T cells can provide cells with greater potency and persistence for enhanced therapeutic efficacy. This technology also has the potential for specific targeting of Notch agonists to selected T cell subsets using a bispecific targeting agent to restrain differentiation and develop highly effective cells for immunotherapy of cancer, infectious diseases, and autoimmunity. See, e.g., WO 2018/017827, incorporated herein in its entirety.

In accordance with the foregoing, the present disclosure is generally directed to, inter alia, methods that promote relatively undifferentiated states of T cells that comprise stimulating Notch signaling in the T cells, compositions comprising the resulting T cells, and therapeutic methods of using the resulting T cells.

In one aspect, the disclosure provides a method of culturing a T lymphocyte (“T cell”) in vitro. The method comprises exposing the T cell to a Notch receptor agonist for a time sufficient to induce Notch receptor signaling in the cell. In another aspect, the disclosure provides a method of culturing T cells in vitro, comprising exposing a population of T cells to a medium comprising a Notch receptor agonist for a time sufficient to induce Notch receptor signaling in the cell. Various elements of these methods are described in more detail below.

In some embodiments, the T cell or T cell population is not cultured in the presence of an antigen-presenting cell (APC). In some embodiments, the Notch receptor agonist is not expressed on a cell present in the medium. In some embodiments, the T cell or T cell population is not co-cultured in the presence of an antigen-presenting cell (APC) expressing a Notch receptor agonist. For example, in some embodiments, the Notch receptor agonist is not expressed on an APC in the medium. In some embodiments, the T cell or T cell population is not cultured in the presence of an antigen-presenting cell (APC) expressing Delta like ligand 4 (DLL4).

T Cells

The T cells are now described. While the description is generally in the context of a single T cell in the method, it will be understood that the description can also apply in aggregate to a population of T cells unless explicitly stated otherwise.

In some embodiments, the T cell is characterized by expression of CD3 and CD28 on its surface (CD3 positive (+) and CD28 positive (+)). The T cell can be in any developmental state (i.e., T cell subset), such as selected from the subsets including a naïve T cell (T_{N}), a stem central memory T cell (T_{SCM}), a central memory T cells (T_{CM}), an effector memory T cell (T_{EM}), and an effector T cell (T_{E} or T_{EFF}).

In some embodiments, the T cell is characterized as less differentiated, i.e., with a relatively high proliferative capacity and low effector function capacity. In some embodiments, performance of the disclosed methods, including exposing T cells to a Notch receptor agonist promotes maintenance of a less differentiated state. For example, the T cell can be a naïve T cell (T_{N}) or a memory stem T cell (T_{SCM}). In a specific embodiment, the T cell is a naïve T cell (T_{N}).

The T cell can also be characterized by the presence or absence of particular surface markers. For example, the T cell can be positive for one or more of the following surface markers: CCR7, CD62L, CD45RA, CD27, CD28, CD95, and CD127. In one embodiment, the T cell is positive for at least CD62L. In one embodiment, the T cell is positive for at least CD62L and CD45RA.

In some embodiments, the T cell is negative for one or more of the following surface markers: CD45RO, CD95, PD-1, Lag-3, and CD25. In one embodiment, the T cell is negative for at least CD45RO and CD95.

In one embodiment, the T cell is CD62L+, CD45RA+, and CD45RO−. In one embodiment, the T cell is CD62L+, CD45RA+, CD45RO− and CD95−. In another embodiment, the T cell is CD62L+, CD45RA+, CD45RO− and CD95+.

In some embodiments, the T cell is characterized as being “intermediately differentiated”, i.e., with a relatively reduced proliferative capacity and higher effector functional capacity compared to T_{N} or T_{SCM}. In some embodiments, performance of the disclosed methods, including exposing T cells to a Notch receptor agonist promotes maintenance of an intermediately differentiated state, and in some further embodiments a reversion to a less differentiated state. In some embodiments, the T cell is a central memory T cells (T_{CM}) or an effector memory T cell (T_{EM}), and performance of the method promotes reversion of the T cell to a less differentiated state (as described above.) In some embodiments, the “more differentiated” T cell can be characterized by expression of CD95 and CD45RO. In some embodiments, the T cell is characterized by lack of expression of CD45RO. In some embodiments, the T cell is characterized by lack of expression of CD62L. In one embodiment, the “more differentiated” T cell is CD62L+, CD45RA−, CD45RO+ and CD95+. In another embodiment, the “more differentiated” T cell is CD62L−, CD45RA−, CD45RO+ and CD95+.

In some embodiments, the method is performed on a plurality of T cells, which can be referred to as a “batch.” The batch can be sourced initially from a single donor individual or can be a combined batch of T cells sourced initially from a plurality of individuals (typically of the same species). In context of adoptive cell therapies discussed below, the source individual(s) can be the same or different from a subject that receives administration of the T cells or their progeny. Cells are typically removed or isolated from biological samples, e.g., blood samples, obtained from the source individual(s). In certain embodiments the cells are obtained from apheresis or leukapheresis of the source individual(s). One or more processing steps can be included to isolate the relevant lymphocytes from the other components of the biological sample, including centrifugation, washing, and incubation with appropriate reagents. The cells can be further isolated by selection based on surface expression of the appropriate surface markers characteristic of T cells and/or desired T cell differentiated subtypes, as described above. Selection can be accomplished using, for example, immunostaining techniques combined with flow separation, immunoaffinity magnetic beads, immunoaffinity chromatography, and the like.

The T cells can be substantially homogenous, meaning that about 60%, 70%, 80%, 90%, or all (or any % included within the range of about 60% to 100%) have similar marker expression profiles defining a particular subset, lineage, characteristic, and/or differentiation state of the T cells.

However, in some embodiments the plurality of T cells is not pre-selected for any single type of differentiated subtype, but rather is in bulk, i.e. having variation (potentially substantial variation) in T cell subsets or relevant marker profiles. In this regard, the batch can comprise, for example, T cells characterized representing at least two or more of the differentiated subtypes described above. In some embodiments, the batch is predominantly (i.e., over about 50%, 60%, 70%, 80%, 90%, 95%) “less differentiated” (i.e., T_{N} and/or T_{SCM}). In some embodiments, the batch is predominantly (i.e., over about 50%, 60%, 70%, 80%, 90%, 95%) “intermediately differentiated” (i.e., T_{CM} and/or T_{EM}). In some embodiments, the batch is predominantly (i.e., over about 50%, 60%, 70%, 80%, 90%, 95%) more differentiated (e.g., T_{E}). Alternatively, the batch can contain a combination of these differentiated subtypes at any desired proportion and within any acceptable margin of error.

Furthermore, in some embodiments, the individual T cells (or substantially all, e.g., at least about 90% or more) in the plurality of T cells can be either CD4+, CD8+, or CD4+ and CD8+ can be present in combination at any relative ratio. For example, in some embodiments, the plurality of T cells are selected to comprise either CD4+ T cells to the exclusion of CD8+ cells, CD8+ cells to the exclusion of CD4+, or they have both CD4+ T cells and CD8+ T cells. As described in more detail below in EXAMPLE 4, it was determined that exposure to Notch receptor agonists has positive effects on both CD4+ T cells and CD8+ T cells, the profiles of the effects are distinct. It was demonstrated that while CD4+ T cells are induced by Notch signaling to have greater proliferation, they exhibit less antitumor effect as compared to CD8+ T cells that are independently induced by Notch signaling in the same manner. However, it was determined that combining the Notch induced CD4+ T cells and CD8+ T cells have a synergistic effect, leading to significantly enhance proliferation rates, resistance to exhaustion, and antitumor effects. Moreover, it was determined that exposure specifically of the CD4+ T cells to a Notch agonist had the greatest influence on proliferation resistance to exhaustion, and antitumor effect. Accordingly, in some embodiments the plurality of T cells exposed to Notch receptor ligand comprises or consists of CD4+ T cells. The Notch-stimulated CD4+ T cells can subsequently be combined with, or coordinately administered with, CD8+ T cells that have not been exposed to Notch receptor ligand.

Additional exemplary combinations of the starting population of T cells exposed to a Notch receptor agonist include T cells having a ratio of CD4+:CD8+ cells from about 20:1 to about 1:20. For example, the plurality of T cells can have a ratio CD4+:CD8+ cells from about 20:1, from about 19:1, from about 18:1, from about 17:1, from about 16:1, from about 15:1, from about 14:1, from about 13:1, from about 12:1, from about 11:1, from about 10:1, from about 9:1, from about 8:1, from about 7:1, from about 6:1, from about 5:1, from about 4:1, from about 3:1, from about 2:1, from about 1:1, from about 1:2, from about 1:3, from about 1:4, from about 1:5, from about 1:6, from about 1:7, from about 1:8, from about 1:9, from about 1:10, from about 1:11, from about 1:12, from about 1:13, from about 1:14, from about 1:15, from about 1:16, from about 1:17, from about 1:18, from about 1:19, and from about 1:20.

Selection of desired cells based on surface markers can be accomplished, for example, using immunostaining-based techniques as described above.

In some embodiments, the method is a method of culturing naïve T (T_{N}) cells in vitro, comprising exposing a population of naïve T (T_{N}) cells to a medium comprising Notch receptor agonist for a time sufficient to induce Notch receptor signaling in the cell. The population of T_{N} cells can comprise at least about 40%, at least about 50%, at least about 60%, at least about 70%, at least about 80%, at least about 85%, at least about 90%, at least about 95%, at least about 96%, at least about 97%, at least about 98%, at least about 99% or about 100% of T_{N} cells. In some embodiments, the population comprises about 40% to about 90%, about 50% to about 90%, about 60% to about 90%, about 70% to about 90%, about 80% to about 90%, about 40% to about 80%, about 50% to about 80%, about 60% to about 80%, about 70% to about 80%, about 40% to about 70%, about 50% to about 70%, about 60% to about 70%, about 40% to about 60%, about 50% to about 60%, or about 40% to about 50% of T_{N} cells. In some embodiments, the T_{N} cell is further characterized as CD62L+, CD45RA+, CD45RO−, CD95−, and/or CCR7+.

Notch Receptor Agonist

Notch receptors in mammals include Notch1, Notch2, Notch3, and Notch4, and homologs of which are known and readily ascertainable by persons of ordinary skill in the art for humans, rodents, and other species. For example, representative amino acid sequence for human Notch1 is provided in Genbank Accession No. P46531, which is incorporated herein by reference in its entirety, and is set forth herein as SEQ ID NO:7. Other Notch receptors are well-known and readily identifiable. A representative example of human Notch2 is provided in Genbank Accession No. NP_077719.2, which is incorporated herein by reference in its entirety, and is set forth herein as SEQ ID NO:8. A representative example of human Notch3 is provided in Genbank Accession No. AAB91371.1, which is incorporated herein by reference in its entirety, and is set forth herein as SEQ ID NO:9. A representative example of human Notch4 is provided in Genbank Accession No. AAC63097.1, which is incorporated herein by reference in its entirety, and is set forth herein as SEQ ID NO:10. Each of the above accession numbers is incorporated herein by reference.

The disclosed methods incorporate exposing one or more T cells to a Notch receptor agonist. As described above, the Notch receptors are integrated into a highly conserved signaling pathway that facilitates cell to cell signaling. As described below in more detail, the inventors have established that exposure of T cells to Notch receptor agonist that stimulates Notch cell-signaling results in the T cells maintaining a less differentiated state with enhanced survival and persistence when administered in vivo. This in turn manifests in enhanced anti-tumor activity with reduced susceptibility to exhaustion.

The term “Notch receptor agonist” is a molecule that specifically binds to a Notch receptor in a manner that functionally results in Notch signaling when the Notch receptor is expressed on the surface of a cell. For example, the Notch receptor agonist of the disclosure includes any canonical or noncanonical ligand to mammalian Notch receptor (e.g., a ligand that binds to a mammalian Notch1, Notch2, Notch3, or Notch4 receptor), or any affinity reagent that specifically binds to a mammalian Notch receptor, and that has agonist functionality resulting in Notch receptor signaling in the cell expressing the Notch receptor. The Notch receptor agonist can also referred to generally as a “Notch ligand”, “Notch ligand”, “Notch agonist,” “Notch receptor agonist ligand”, and the like.

As used herein the term “Notch signaling” or other references to the function of Notch receptor when contacted with a Notch receptor agonist refers to the cell-signaling cascade that occurs from the proteolytic cleavage of the expressed mature Notch receptors in a cell membrane. As indicated above, Notch signaling is initiated by proteolytic cleavage at an S2 site near the C-terminal end of the NECD. In a resting or non-signaling state the S2 cleavage site is protected by a closed confirmation of the negative regulatory region (NRR), which directly occludes the cleavage site and prevents access to the protease. See, e.g., Gordon et al., “Structure of the Notch1-negative regulatory region: implications for normal activation and pathogenic signaling in T-ALL,” Blood, 113(18):4381-4390 (2009), incorporated herein by reference in its entirety. Ligand-mediated activation of signaling occurs when the ligand binds to the extracellular domain in a manner resulting in a conformational change that exposes the S2 cleavage site in the NRR of the extracellular domain. The conformational change is not necessarily automatic, but occurs when the ligand or other binding molecule imposes a force or strain on the Notch receptor such that the closed confirmation of the NRR is altered. Accordingly, a Notch receptor agonist not only binds to the NECD, but binds in a manner that applies sufficient force, or “tug”, to induce the conformational change required to expose the S2 cleavage site. Exposure of the S2 cleavage site permits proteolytic cleavage and subsequent intracellular signaling. The functionality of a “tug” in any Notch ligand or binding molecule, such as described below, can be accomplished by appropriately presenting the Notch receptor agonist to the cell expressing the Notch receptor, as described in more detail below.

Notch signaling can be monitored by measuring downstream gene products resulting from Notch activation, such as Hes1 expression. Alternatively, reporter systems are available to indicate Notch signaling, such as the CHO-K1 Notch reporter system. See, e.g., Sprinzak, D., et al. “Cis-interactions between Notch and Delta generate mutually exclusive signalling states,” Nature 465(7294):86-90 (2010), incorporated herein by reference in its entirety.

Nonlimiting examples of Notch receptor agonists encompassed by this disclosure and are described.

The Notch receptor agonist can be or can comprise canonical mammalian Notch receptor ligands, or a Notch-binding domain thereof, that have been demonstrated to induce Notch signaling. Canonical Notch ligands in mammals include Jagged proteins (e.g., Jagged1 and Jagged2) and Delta proteins (e.g., DLL1, DLL3, DLL4; where DLL is an acronym for “Delta Like Ligand”), each of which are well-known and are contemplated and encompassed by this disclosure. As non-limiting examples, representative canonical Notch ligand sequences comprise sequences disclosed in GenBank Accession No. AAC51731 (Jagged1; set forth herein as SEQ ID NO:11), GenBank Accession No. AAD15562 (Jagged2; set forth herein as SEQ ID NO:12), GenBank Accession Nos. ABC26875 (partial sequence) or NP005609 (DLL1; set forth herein as SEQ ID NOS:13 and 14, respectively), GenBank Accession Nos. NP_982353.1 or NP_058637.1 (DLL3; set forth herein as SEQ ID NOS:15 and 16, respectively), and NP_061947.1 (DLL4; set forth herein as SEQ ID NO:17) (the sequence of each accession number incorporated herein by reference), homologs, or functional (Notch binding) variants, fragments, or derivatives thereof. These canonical Notch ligands, collectively referred to as DSL ligands, typically contain an N-terminal region, a DSL domain, and at least two EGF-like repeats, which are necessary for interaction with EGF repeats 11 and 12 of Notch receptors. Accordingly, in some embodiments, the Notch receptor agonist of the disclosure comprises an extracellular domain of a Delta protein or a Jagged protein, such as vertebrate (e.g., mammalian) or invertebrate Delta or Jagged proteins, as described herein. A 2.3 angstrom resolution crystal structure of interacting regions of Notch1-DLL4 indicates the structural components of the ligand-receptor complex important for binding. See Luca, V. C., et al., “Structural Basis for Notch1 Engagement of Delta-Like 4,” Science 347(6224):847-853 (2015). Luca, et al., (2015), which is incorporated herein in its entirety, further discloses modifications in the wild-type DLL4 that enhance binding affinity to the receptor, thus further illuminating required and critical domains in a canonical Notch ligand required for binding to the Notch receptor. Accordingly, a person of ordinary skill in the art can readily identify minimal Notch binding domains from known or putative Notch ligands. In some embodiments, the Notch receptor agonist is not DLL1. In further embodiments, the T cell or population of T cells are not co-cultured with any other cell that expresses DLL1.

In some embodiments, Notch receptor agonist of the disclosure can comprise polypeptide sequences with one or more mutations in a wild-type sequence resulting in modified (e.g., enhanced) affinity for the Notch receptor. For example, as demonstrated in the E12 variant of rat DLL4 disclosed in Luca, V. C., et al., “Structural Basis for Notch1 Engagement of Delta-Like 4,” Science 347(6224):847-853 (2015), incorporated herein in its entirety, mutations of G28S, F107L, L206P, N118I, I143F, H194Y, K215E, individually or in any combination, can enhance affinity of binding. Accordingly, in an illustrative, non-limiting embodiment, the Notch binding domain can comprise an amino acid sequence with at least 80% (such as about 80%, 81%, 82%, 83%, 84%, 85%, 86%, 87%, 88%, 89%, 90%, 91%, 92%, 93%, 94%, 95%, 96%, 97%, 98%, and 99%) sequence identity to the sequence set forth in SEQ ID NO:2. SEQ ID NO:2 is a wild-type polypeptide sequence of a rat DLL4 fragment corresponding to the MNNL to EGF2 domains (i.e., amino acid positions 27 to 283) of the full-length precursor. The full length rat DLL4 precursor is set forth herein as SEQ ID NO:1. In some embodiments, the Notch binding domain comprises a polypeptide with a sequence that includes at least one substitution at an amino acid position selected from: 28, 43, 52, 96, 107, 118, 143, 146, 183, 194, 206, 215, 223, and 257 (the positions are numbered with respect to positions within the reference sequence set forth in SEQ ID NO:1 and corresponding homologous positions in other DLL proteins can be readily ascertained by alignment). In certain embodiments, the at least one substitution enhances affinity. In some embodiments, the at least one substitution is selected from: G28S, M/V43I, P52S, S96I, F107L, N118I, I143F/T, Q146K, S183N, H194Y, L206P, K215E, L223R, and N257K, or a similar substitution at a corresponding amino acid residue in a homologous sequence. In some instances, the high affinity Notch receptor ligand comprises at least 2, 3, 4, 5, 6, 7, 8, 9, 10 or more of the substitutions set forth above. Any combination of substitutions as set forth above is contemplated. Examples of specific combinations of substitutions include, but are not limited to: (i) P52S, F107L, L206P; (ii) F107L, L206P, N257K; (iii) F107L, L223R, N257K; (iv) G28S, M43I, F107L, N118I; (v) G28S, F107L, N1181, Q146K, H194Y, L206P, K215E; (vi) G28S, F107L, N118I, I143F, H194Y, L206P, K215E; (vii) G28S, M43I, S961, N118I, I143T, S183N, H194Y, L206P, K215E; (viii) G28S, F107L, L206P; and (ix) G28S, F107L, L206P, N257K (or a similar substitution at a corresponding amino acid residue in a homologous sequence). An exemplary DLL4 encompassed by the disclosure is available from R&D Systems (catalog no. 1506-D4/CF).

Also disclosed in Luca, et al. (2015), mutations to Jagged proteins could be mapped to the sequence of DLL4 indicating important residues on this ligand for contact and binding on the Notch receptor. Thus, the Notch receptor agonist of the disclosure can comprise an amino acid sequence with at least 80% (such as about 80%, 81%, 82%, 83%, 84%, 85%, 86%, 87%, 88%, 89%, 90%, 91%, 92%, 93%, 94%, 95%, 96%, 97%, 98%, and 99%) sequence identity to the sequence set forth in SEQ ID NO:4, which sets forth the amino acid sequence corresponding to the amino acids 32 to 295 of the full wild type rat Jagged1 polypeptide. The full wild type rat Jagged1 polypeptide sequence is set forth in SEQ ID NO:3. In additional embodiments, the Notch receptor agonist of the disclosure can comprise at least one substitution at an amino acid position selected from 100 and 182, with reference to positions in SEQ ID NO:3 (although not requiring the entire sequence; homologous positions in other DLL proteins can be readily ascertained by alignment). In certain embodiments, the at least one substitution is selected from: P100H, Q183P, and a combination thereof. Alternatively, in homologous sequences, the at least one substitution can be at the corresponding amino acid residue position(s) in the homologous sequence.

In other embodiments, the Notch receptor agonist of the disclosure can comprise an amino acid sequence with at least 80% (such as about 80%, 85%, 90%, 91%, 92%, 93%, 94%, 95%, 96%, 97%, 98%, 99%, or 100%) sequence identity to the sequence set forth in SEQ ID NO:5 or 6, which set forth the amino acid sequence of the extracellular Notch-binding regions of representative human Jagged2 (Genbank Accession No. AAD15562.1) and human Delta like 1 (DLL1; Genbank Accession No. NP005609.3), respectively. In view of the above structural studies and other available data, persons of ordinary skill in the art can readily ascertain permissible variations in the reference sequences that still result in functional binding to the Notch receptors.

In addition to canonical Notch receptor ligands or Notch receptor-binding domains thereof, the Notch receptor agonist of the disclosure can comprise a Notch binding domain (or a Notch-binding derivative or fragment thereof) of any non-canonical Notch receptor ligand so long as it has or retains agonist activity. See, e.g., Hu, Q., et al., “F3/contactin acts as a functional ligand for Notch during oligodendrocyte maturation,” Cell 115(2):163-175 (2003); Schmidt, M. H., et al., “Epidermal growth factor-like domain 7 (EGFL7) modulates Notch signalling and affects neural stem cell renewal,” Nat Cell Biol 11(7):873-880 (2009); and D'Souza, B., et al., “Canonical and non-canonical Notch ligands,” Curr Top Dev Biol 92:73-129 (2010), each of which is incorporated herein by reference in its entirety. Any fragments or derivatives that retain the ability to bind and activate the target Notch receptor, which can be readily assayed by persons of ordinary skill in the art, are encompassed by the present disclosure. See, e.g., assays disclosed elsewhere herein. In some embodiments, the derivative can comprise an amino acid sequence with at least 80% (such as about 80%, 81%, 82%, 83%, 84%, 85%, 86%, 87%, 88%, 89%, 90%, 91%, 92%, 93%, 94%, 95%, 96%, 97%, 98%, and 99%) of the sequence of the source Notch binding domain of the non-canonical Notch receptor ligand.

While the above description includes examples of rat or human Notch ligands, it will be appreciated that the indicated mammalian sources for Notch ligands serving as a Notch receptor agonist can include the non-limiting examples of human, non-human primates (including, e.g., apes and monkey, and the like), rodent (including, e.g., rat, mouse, guinea pig, and the like), dog, cat, horse, cow, pig, sheep, and the like. Non-mammalian Notch ligands, such as Drosophila Serrate and Delta, are also well-known and are encompassed by the present disclosure. As indicated, the Notch signaling system is highly conserved and, thus, homologous sequence positions among the Notch receptors and respective Notch ligands are readily ascertainable by persons of ordinary skill in the art. Furthermore, agonist activity of the Notch receptor ligands can be readily ascertainable by, for example, performing an assay to detect Notch-induced transcriptional activity of the cell (e.g., Hes gene transcription, as described below).

In addition to canonical or noncanonical Notch ligands or Notch binding domains thereof, as described above, the Notch receptor agonist encompassed by the disclosure can be or comprise an affinity reagent designed to bind a Notch receptor with agonist functionality. As used herein, “affinity reagent” refers to any molecule that can bind a target antigen, in this case relevant domains of the NECD of the Notch receptor, with elevated affinity (i.e., detectable over background) and thereby induce Notch signaling. The binding affinity for Notch receptor can be specific or selective, but need not be so. As used herein, the term “specifically bind” or variations thereof refer to the ability of the affinity reagent component to bind to the antigen of interest (e.g., Notch receptor), without significant binding to other molecules or the NRR domain (described below), under standard conditions known in the art. The antigen-binding molecule can bind to other peptides, polypeptides, or proteins, but with lower affinity as determined by, e.g., immunoassays, BIAcore, or other assays known in the art. However, affinity reagent preferably does not substantially cross-react with other antigens or the NRR domain.

Exemplary, non-limiting categories of affinity reagent include antibodies, an antibody-like molecule (including antibody derivatives and antigen (i.e., Notch)-binding fragments thereof), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional Notch-binding domain or fragment thereof. These affinity reagents are described in more detail below in the “Additional definitions” section. The agonist affinity reagents can be generated through application of known techniques to bind to the NECD of the Notch receptors, described above.

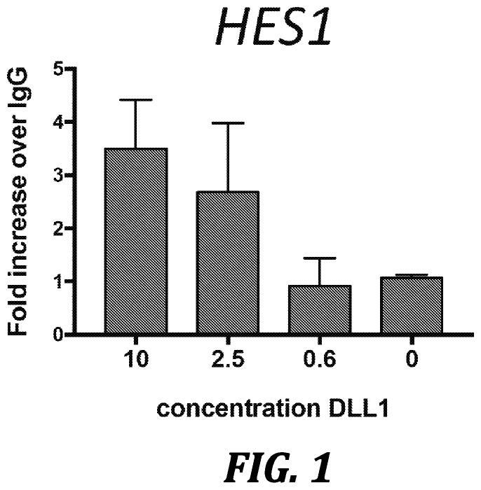

The agonist affinity reagents bind Notch “with agonist functionality” when they bind to the NECD without interfering with the functionality of the negative regulatory region (NRR) to undergo a conformational change. As described above, the NECD contains an extended section containing EGF-like repeats, followed by the NRR. The agonist affinity reagents bind epitopes within the NECD of the Notch receptor except for any epitopes in the NRR. This permits the NRR, upon binding of the agonist affinity reagent, to alter its closed confirmation, thereby revealing the S2 proteolytic site to permit signaling. The domain in the NECD available for binding to confer signaling capability extends generally from the N-terminus to just before the beginning of the NRR. See, e.g. FIG. 1 of Gordon et al., Blood, 113(18):4381-4390 (2009), incorporated herein by reference in its entirety. This target domain in the NECD for binding includes the EGF-like repeat motifs. In some embodiments, the domain containing epitopes to which the agonist affinity reagent binds is in a region corresponding to e.g. amino acid position 1 to about amino acid position 1446 of SEQ ID NO:7 (for Notch1 receptor); amino acid position 1 to about amino acid position 1421 of SEQ ID NO:8 (for Notch2 receptor); amino acid position 1 to about amino acid position 1383 of SEQ ID NO:9 (for Notch3 receptor); and amino acid position 1 to about amino acid position 1170 of SEQ ID NO:10 (for Notch4 receptor). Subdomains of the NECD from other Notch homologues that exclude the NRR can be readily determined based on comparison to the above amino acid positions and in the alignment disclosed in Gordon et al., Blood, 113(18):4381-4390 (2009).

As indicated, agonist affinity reagents include antibodies or Notch receptor-binding fragments and derivatives thereof. Non-limiting, exemplary antibodies that bind to Notch encompassed by the present disclosure include the N1 anti Notch1 antibody (HMN1-519, Biolegend catalog #352104) as well as the Notch1 agonistic antibodies disclosed in, e.g., Wu et al., Nature 464:1052-57 (2010); U.S. Pat. Nos. 9,683,039 B2, 10,370,643 B2, 10,208,286 B2, 9,221,902 B2, 6,689,744 B2, 6,090,922 A, or 6,149,902 A; or U.S. Publication No. 2011/0286916 A1, or 2005/0222074 A1; each of which is incorporated by reference herein in its entirety. Non-limiting exemplary Notch2 agonistic antibodies include but are not limited to HMN2-25 (also referred to herein as “N2”; Biolegend catalog #348301), or an antibody disclosed in, e.g., Wu et al., Nature 464:1052-57 (2010); U.S. Pat. Nos. 9,683,039 B2, 10,370,643 B2, 10,208,286 B2, 9,221,902 B2, 6,090,922 A, or 6,149,902 A; or U.S. Publication No. 2005/0222074 A1; each of which is incorporated by reference herein in its entirety. Non-limiting examples of Notch3 agonistic antibodies include but are not limited to an antibody disclosed in, e.g., Machuca-Parra et al., J. Exp. Med. 214(08):2271-82 (2017); Li et al., J. Biol. Chem. 283(12):8046-54 (2008); U.S. Pat. Nos. 9,518,124 B2, 9,089,556 B2, 9,221,902 B2, 8,513,388 B2, 6,090,922 A, or 6,149,902 A; U.S. Publication No. 2008/0118520 A1, 2008/0131908 A1, or 2005/0222074 A1; or International Application No. WO 2010/141249 A2; each of which is incorporated by reference herein in its entirety. Non-limiting examples of Notch4 agonistic antibodies include but are not limited to an antibody disclosed in, e.g., U.S. Pat. Nos. 10,227,567 B2, 9,221,902 B2, 6,090,922 A, or 6,149,902 A; or U.S. Publication No. 2005/0222074 A1; each of which is incorporated by reference herein in its entirety.

As indicated above Notch signaling induced by an agonist requires not only sufficient agonist binding affinity, but the binding must be in a manner that applies a sufficient mechanical force or “tug”, to induce the required conformational change allowing proteolytic cleavage at the S2 cleavage site. Thus, the Notch receptor agonist must be appropriately presented to the Notch expressing cell such that the required force or tug can be applied.

In some embodiments, the Notch receptor agonist is immobilized in a manner that does not negatively affect its ability to stimulate Notch signaling when a T cell is contacted thereto. The Notch receptor agonist can be immobilized to a surface, such as the surface of a tissue culture plate, or on particles or beads amenable to mixture in a culture medium. Immobilization, for example on a surface of the plate or well, will prevent the T cell expressing Notch from rolling away, thereby creating tension on the ligand-receptor complex sufficient to alter Notch confirmation. Similarly, the attachment of particles or beads, or other bulky carriers, to the ligand provides bulk or weight that similarly creates tension on the ligand-receptor complex. In other embodiments the Notch agonist is attached to a soluble scaffold, such as a hydrogel. Similar to other services or scaffolds, this provides stability to the ligand and prevents the cells from rolling away, thereby creating tension on the ligand-receptor complex. The capacity for solubility of the scaffold, permits imposition of the temporal limit to the signaling; as the scaffold dissolves into the culture the attached ligands are released and lose the capacity to stimulate Notch signaling. In yet another embodiment, the Notch agonists can be presented in multimer form. Multimeric ligands can bind to a plurality of Notch expressing cells simultaneously. The bulk and counterweight applied by each of the plurality of bound Notch-expressing cells create the tension or “tug” required to induce the conformational change necessary to initiate proteolytic cleavage and subsequent signaling. In some embodiments, the Notch receptor agonist is not expressed on or otherwise presented by a cell that is co-cultured with the T cell or population of T cells. In some embodiments, the T cell or population of T cells is not cultured in the presence of an antigen presenting cell (APC) expressing a Notch receptor agonist. For example, in some embodiments, the Notch receptor agonist is not expressed on an APC in the medium. In some embodiments, the T cell or population of T cells is not cultured in the presence of an antigen presenting cell (APC) expressing Delta like ligand 4 (DLL4).

While the various Notch receptor agonist described above have different affinities for different Notch receptors (e.g. Notch1, Notch2, Notch3, Notch4, etc.), the functional differences among the selection of appropriate Notch receptor agonist is merely quantitative depending on the prevalence of the particular Notch receptor(s) in the target cells. Lower affinity generally results in lower total signaling in a batch exposure of cells. Accordingly, even Notch receptors with a relatively low affinity for prevalent Notch receptor(s) in the desired target cells can be used. In some embodiments, this is preferred if lower levels of signaling is desired. Alternatively, the relatively low affinity can be compensated for by, e.g., increasing the density of the presented agonist, and or increasing the level of costimulatory molecules and/or cytokines. By adjusting such factors as choice of Notch receptor agonist (or agonists), the density of Notch receptor agonist (or agonists), and choice and concentration cofactors, and/or cytokines, the intensity of Notch signaling can be controlled.

In some embodiments, the Notch receptor agonist is contacted with a batch of T cells at a predetermined density or concentration in the medium to facilitate controlled degree of Notch signaling in a batch of T cells. For example, in some embodiments the Notch receptor agonist is immobilized at a concentration of about 0.01 μg/ml to about 10 μg/ml, such as about 0.025 μg/ml, about 0.05 μg/ml, about 0.1 μg/ml, about 0.25 μg/ml, about 0.5 μg/ml, about 0.75 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml. In other embodiments the Notch receptor agonist is immobilized at a concentration of up to about 100 μg/ml, such as about 10 μg/ml, about 15 μg/ml, about 20 μg/ml, about 25 μg/ml, about 30 μg/ml, about 35 μg/ml, about 40 μg/ml, about 45 μg/ml, about 50 μg/ml, about 55 μg/ml, about 60 μg/ml, about 65 μg/ml, about 70 μg/ml, about 75 μg/ml, about 80 μg/ml, about 85 μg/ml, about 90 μg/ml, about 95 μg/ml, and about 100 μg/ml, or any range therein. Typically, the higher affinity of the Notch receptor agonist to the Notch receptor expressed by the target T cell, the lower density or concentration of the Notch receptor agonist will be required to achieve a comparable Notch signaling. In one exemplary embodiment, the anti-Notch1 antibody, N1, can be presented in a concentration of about 0.25 μg/ml to about 2.5 μg/ml, such as 0.25 μg/ml, about 1 μg/ml, about 2.5 μg/ml. In another exemplary embodiment, the canonical Notch ligand DLL1, or Notch-binding domain thereof, can be presented in a concentration of about 2.5 μg/ml to about 10 μg/ml.

In some embodiments, combinations of two or more Notch receptor agonist, such as described above, are exposed to the target T cell in a matter sufficient to impose a mechanical force or “tug” to induce a conformational change and initiate signaling.

In some embodiments, the batch of T cells is exposed to the Notch receptor agonist at a cell concentration that does not exceed a threshold where significant cell-to-cell Notch signaling is induced. Typically the maximum amount of cells is added that can be bound to the immobilized substrate in the culture conditions.

Medium

The method is typically performed under culture conditions that support maintenance of cell viability and proliferation of cells, as can be readily determined and applied by a person of ordinary skill in the art.

As indicated above, the medium can be in a container, such as a flask or plate, which has the Notch receptor agonist immobilized thereto. Alternatively, a liquid medium contained in the container can comprise the Notch receptor agonist distributed therein. In some embodiments, the Notch receptor agonist(s) is/are immobilized to a particle or bead surface, wherein the particles or beads are distributed in the medium.

In some embodiments, the medium comprises additional components or conditions that promote maintenance and/or stimulation of T cells to result in T cells that maintain or revert to a relatively undifferentiated state with a higher proliferative capacity, as described above. The components and conditions can promote proliferation, expansion, and activation of the cells without promoting progression towards more differentiated states. Such conditions can also be designed to prime the cells for genetic manipulations.

Exemplary culture factors can include e.g., nutrients, amino acids, antibiotics, ions, and/or stimulatory factors, such as cytokines, chemokines, antigens, binding partners, fusion proteins, recombinant soluble receptors, and any other agents designed to facilitate activation the T cells.

Various additional conditions, such as oxygen and carbon dioxide levels, temperature, time of culture, light exposure, and the like, can be controlled to facilitate or optimize performance of the methods.

In some embodiments, factors stimulating the TCR/CD3 signaling cascade in the T cells can be included in the culture. For example, binding (affinity) agents, such as antibodies or antibody fragments, specific for CD3, CD28, and/or 4-1BB can be included to help induce T cell stimulation and proliferation. Other exemplary T cell stimulatory factors encompassed by the present disclosure known in the art are encompassed by the present disclosure. Such factors can be included on solid supports, such as beads such that their presence can be controlled. For example, the assays described below include the use of beads (i.e., DYNABEADS®; ThermoFisher)) displaying anti-CD3 and anti-CD28 antibodies. As described below, such reagents may also be useful for eventual extraction and selection of desired T cells. Alternatively or additionally, the culture can contain cells expressing Notch ligands, as described above, and/or one or more T cell stimulatory or costimulatory molecules. For example, in exemplary cell lines useful for this purpose is OP9 (ATCC® CRL-2749™), which are derived from mouse bone marrow stromal cells in which can induce T cell stimulation and proliferation. In other embodiments, cells expressing Fc receptor can be included. However, in other embodiments, the T cells (e.g. naïve T cells) are not co-cultured with additional cells. In some embodiments, the T cells (e.g. naïve T cells) are not co-cultured with antigen presenting cells. In some embodiments, the T cells (e.g. naïve T cells) are not co-cultured with bone marrow cells. In some embodiments, the T cells (e.g. naïve T cells) are not co-cultured with OP9 cells. In additional embodiments, the T cells (e.g. naïve T cells) are not co-cultured with OP9-DL1 cells. In yet other embodiments, the T cell or population of T cells (e.g. naïve T cells) is not co-cultured with antigen presenting cells (APCs) expressing a Notch receptor agonist. In some embodiments, the T cell or population of T cells (e.g. naïve T cells) is not co-cultured with APCs expressing DLL4.

Various cocktails of signaling factors, e.g., cytokines or biologically active fragments thereof, can be included in the culture conditions for part or all of the culture phase to promote activation and proliferation of the T cells. In some embodiments, the signaling factor is a ligand that specifically binds to a cytokine receptor selected from among IL-2R, IL-1R, IL-15R, IFN-γR, TNF-αR, IL-4R, IL-10R, Type I IFNR, IL-12R, IL-15R, IL-17R, TNFR1 and TNFR2. Various cytokine cocktails with predetermined concentrations of one or more cytokines are contemplated and can include one or more exemplary cytokines such as IL-1, IL-lb, IL-2, IL-4, IL-6, IL-7, IL-10, IL-12, IL-15, IL-17, IL-21, IL-23, IL-27, IFN-γ, TNF-α, TGF-β, or biologically active fragments thereof, in any combination, at appropriate concentrations that can be readily determined by persons of ordinary skill in the art. In some embodiments, the culture medium comprises one or more cytokines promoting a Th1 profile, such as IFN-γ and/or IL-12. In other embodiments, the culture medium comprises cytokines promoting a Th17 profile, such as TGF-β, IL-6, IL-21, and/or IL-23 in any combination. For example, in one embodiment IL-2 can be used at concentrations of at least about 10 units/ml.

In some embodiments, the culture conditions can also comprise the inclusion of feeder cells, for example non-dividing peripheral blood mononuclear cells (PBMCs). The PBMCs can be irradiated to prevent cell division of the feeder cells. The feeder cells can be included at an amount to provide more than 2:1 (e.g., 2:1, 3:1, 5:1, 10:1, 20:1, 50:1 or higher) ratio of feeder cells to initial T cells in the culture.

The one or more T cells are exposed to the Notch receptor agonist for a time sufficient to induce Notch signaling in the cell. It will be understood that the time of exposure can be influenced by the density or concentration of the Notch receptor agonist and/or the plurality of T cells, the identity of the T cell subtypes, or the source of T cells. In some embodiments, the exposure period can be between about 12 hours to about 20 days, such as between about 1 day and about 15 days, between about 2 days and about 12 days, between about 2 days and about 10 days, between about 2 days and about 8 days, between about 3 days and about 7 days, between about 3 days and about 6 days, and between about 4 days and about 5 days. In some embodiments, the one or more T cells are exposed to the Notch receptor agonist for 1, 2, 3, 4, 5, 6, 7, 8, 9, 10, 11, 12, 13, 14, 15, or more days.

In other embodiments, such as in embodiments of culturing a population of T cells (e.g., T_{N} cells), the exposing lasts for a period (“the exposure time”) of at least about 12 hours, at least about one day, at least about two days, at least about three days, at least about four days, at least about five days, at least about six days, at least about a week, at least about eight days, at least about nine days, at least about ten days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about a month. In some embodiments, the exposure time is between one day to 15 days or between two days to 10 days. In embodiments of culturing a population of T_{N} cells , the percentage of the T_{N} cells in the population does not change after the exposing. In some embodiments, the percentage of the T_{N} cells in the population changes, after the exposing, in less than about 1%, less than about 2%, less than about 5%, less than about 10%, less than about 15%, less than about 20%, less than about 25%, less than about 30%, less than about 35%, less than about 40%, less than about 45%, or less than about 50%. In some embodiments, the percentage of the T_{N} cells in the population is: (i) at least about 40% before the exposing and at least about 40% after the exposing; (ii) at least about 50% before the exposing and at least about 40% after the exposing; (iii) at least about 50% before the exposing and at least about 50% after the exposing; (iv) at least about 50% before the exposing and at least about 60% after the exposing; (v) at least about 60% before the exposing and at least about 50% after the exposing; (vi) at least about 60% before the exposing and at least about 60% after the exposing; (vii) at least about 60% before the exposing and at least about 70% after the exposing; (viii) at least about 70% before the exposing and at least about 60% after the exposing; (ix) at least about 70% before the exposing and at least about 70% after the exposing; (x) at least about 70% before the exposing and at least about 80% after the exposing; (xi) at least about 80% before the exposing and at least about 70% after the exposing; (xii) at least about 80% before the exposing and at least about 80% after the exposing; (xiii) at least about 80% before the exposing and at least about 90% after the exposing; (xiv) at least about 90% before the exposing and at least about 80% after the exposing; (xv) at least about 90% before the exposing and at least about 90% after the exposing; or (xvi) at least about 90% before the exposing and about 100% after the exposing. In some embodiments, the T_{N} cell, the population of T_{N} cells, or one or more progeny cells thereof, maintains at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4.0 fold, at least 4.5 fold, at least 5.0 fold, at least 5.5 fold, at least 6.0 fold, at least 6.5 fold, or at least 7.0 fold less-differentiated state in vivo compared to the T_{N} cell that did not receive the Notch receptor agonist.

In certain embodiments, some methods disclosed herein comprise exposing a T cell or a population of T cells to a medium comprising a Notch receptor agonist for a time sufficient to induce Notch receptor signaling in the cell, wherein the Notch receptor agonist is a peptide ligand and wherein the T cell is not co-cultured with an OP9-DL1 cell or a bone marrow cell expressing DLL1. In some embodiments, the T cell or the population of T cells exhibits at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 250%, or at least about 300% less exhaustion compared to a T cell co-cultured with OP9-DL1 cells.

In some embodiments, the disclosed methods further comprise isolating the T cell(s), and/or progeny thereof, from the Notch receptor agonist and, in some cases, from the initial culture medium. The cells can be assessed for phenotype, such as profiling cell surface expression using flow cytometry, as described in more detail below. The cells can also be appropriately formulated for administration to a subject, as described in more detail below. The cells can also be further combined with additional cells for administration to a subject. For example the cells exposed to Notch receptor agonist can be primarily or predominantly CD4+ cells. After incubation with Notch receptor agonist, CD4+ T cells can be combined with, or administered coordinately with, CD8+ T cells that may or may not have separately been exposed to Notch receptor agonist.

Engineering of Cells

The disclosure also encompasses aspects and embodiments where the T cells comprise genetic modifications that alter the phenotype and/or performance of the cell. For example, in one aspect, a T cell is provided that comprises a heterologous polynucleotide or an expression vector according to the present disclosure, as well as methods for making the same. In some embodiments, wherein the T cell expresses on its cell surface an antigen binding protein encoded by the heterologous polynucleotide. Such T cells are useful for specifically tailored adoptive cell therapeutic techniques. In some embodiments, the disclosed method comprises transducing the T cell with a heterologous nucleic acid to produce a transgenic, or genetically modified, T cell. The genetic modifications can be implemented prior, during, or after the culturing methods described herein. The T cell can be any T cell as described herein. In some embodiments, the cells are cultured for a period of time after the initial exposure of the T cells to the Notch receptor agonist, such as about 1, 2, 3, 4, 5, 6, 7, 8, 9, 10, 11, 12, 13, 14, or more days prior to transduction step. In some embodiments, the cells are cultured for about 1, 2, or 3 days prior to transduction step.

These steps result in recombinant T cells. As used herein, the term “recombinant” refers to a cell, microorganism, nucleic acid molecule, or vector that has been genetically engineered by human intervention—that is, modified by introduction of a heterologous nucleic acid molecule, or refers to a cell or microorganism that has been altered such that expression of an endogenous nucleic acid molecule or gene is controlled, deregulated, deleted, attenuated, or constitutive. Human generated genetic alterations may include, for example, modifications that introduce nucleic acid molecules (which may include an expression control element, such as a promoter) that encode one or more proteins or enzymes, or mutations, such as nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof.

As used herein, the term “transduce” or “transduction” refers to the introduction of heterologous nucleic acid into the T cell to result in a genetic modification in the T cell. Various methods for the introduction of genetically engineered components, for example genetic constructs encoding (new or modified) cytokines and immune receptors (for example CARs and TCRs), are well-understood and can be used with the disclosed methods and compositions.

Such exemplary methods include those for transfer of nucleic acids encoding the receptors, including via viral, e.g., retroviral or lentiviral, transduction, transposons, and electroporation. In some embodiments, the transduction results in the ability of the T cell to express heterologous proteins encoded by the heterologous nucleic acid. In some embodiments, the heterologous nucleic acid (e.g., DNA, RNA, or cDNA) is comprised in a vector, e.g., viral expression vector, that facilitates expression of the heterologous nucleic acid in the nucleus of the cell. In some embodiments, the vector promotes integration of the heterologous nucleic acid in the genome of the cell.

To further illustrate, the term “construct” refers to any polynucleotide that contains a recombinant nucleic acid molecule. As indicated above, the construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

The genetic modification can be operably linked to a nucleic acid sequence that confers functionality, such as a promoter configured to facilitate expression of the new or modified transgenic sequence in the target T cell. Thus, the term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host cell, the vector may replicate and function independently of the host genome, or may, in some instances, integrate into the genome itself. In the present specification, “plasmid,” “expression plasmid,” “virus” and “vector” are often used interchangeably.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, “heterologous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous nucleic acid molecule, construct or sequence may be from a different genus or species. In certain embodiments, a heterologous nucleic acid molecule is added (i.e., is not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by a heterologous polynucleotide introduced into the host cell, even if the host cell encodes a homologous protein or activity.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous nucleic acid molecules encoding desired binding proteins specific for a target antigen peptide (e.g., TCR molecules and/or antibodies). When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

In other embodiments, the genome of the host T cell can be modified by genetic editing techniques to provide a modified or engineered host cell with enhanced activity or altered expression of antigen-specific molecules.

To illustrate, in certain embodiments a chromosomal gene knock-out or gene knock-in can be made by chromosomal editing of a host T cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector (TALE) nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programmed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

In some embodiments, it may be advantageous to reduce expression or activity of autologous genes and proteins that may otherwise have an inhibitory or detrimental effect on the functionality of the engineered T cell. Thus, chromosomal gene knockout can be implemented. The term “chromosomal gene knockout” refers to a genetic alteration in a host cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell. Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest, such as a TCRα chain, TCRβ chain or both, a chimeric antigen receptor, an antibody or antibody components, a modified cytokine, and the like.

In certain embodiments, a chromosomal gene knockout comprises an inhibitory nucleic acid molecule that is introduced into a host cell (e.g., T cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

As indicated above, in some embodiments, the heterologous nucleic acid comprises at least one sequence that encodes, e.g., a signaling factor or an immune receptor. The heterologous nucleic acid can also comprise sequence encoding selection markers, proteins to ensure safety (e.g., susceptible to negative selection).

In some embodiments, the heterologous nucleic acid comprises at least one sequence that encodes a signaling factor, such as proinflammatory cytokines. An exemplary, non-limiting list includes IL-2, IL-12, IL-7, IL-15, and IL-21.

In some embodiments, the heterologous nucleic acid comprises at least one sequence that encodes an immune receptor. The immune receptor can comprise an extracellular domain that, when expressed on the surface of the cell, can bind to an antigen of interest. For example, chimeric antigen receptors (CARs) are encompassed by the aspect of the disclosure. In some embodiments, the extracellular domain of a CAR comprises an antibody fragment (e.g., an scFv) that has specific binding affinity for an antigen of interest. The antigen of interest is typically a marker expressed on the cell surface or extracellular environment. In cancer applications, the marker is ideally unique to the target cancer cells or at least predominantly expressed on the cancer cells compared to healthy tissue. Markers for cancer or infectious agents are known and can be targeted by CAR domains in this aspect. The extracellular domain is linked via a transmembrane and optional spacer domain(s) to at least one intracellular signaling domain that activates the CAR-expressing T cell upon binding to the appropriate ligand. The transmembrane domain can be derived from naturally occurring transmembrane proteins (e.g., alpha, beta or zeta chain of the T-cell receptor, CD2S, CD3epsilon, CD45, CD4, CDS, CDS, CD9, CD 16, CD22, CD33, CD37, CD64, CDSO, CDS6, CD 134, CD137, CD 154, and the like), or can be synthetic with predominantly hydrophobic residues. The intracellular signaling domain can include the CD3 ξ chain, and/or other molecules such as Fc receptor λ. The activating signal initiated by antigen binding eventually results in proliferation and initiation of effector (i.e., cytotoxic) functions of the cell.

Design and introduction of CAR in T cells can include approaches described in, for example, WO200014257; U.S. Pat. No. 6,451,995; US2002131960; U.S. Pat. Nos. 7,446,190; 8,252,592; EP2537416; US2013287748; and WO2013126726,; and/or those described by Sadelain et al., Cancer Discov. 3(4):388-398 (2013); Davila et al., PLoS ONE 8(4):e61338 (2013); Turtle et al., Curr. Opin. Immunol., 24(5):633-39 (2012); and Wu et al., Cancer, 18(2):160-75 (2012); each of which is incorporated herein by reference in its entirety.

Alternatively, the immune receptor is T cell receptor (TCR) that has specific binding properties and signaling activity with respect to a peptide antigen of interest when the peptide is properly complexed with major histocompatibility complex (MHC) proteins. Unlike CARs, which are more restricted to surface antigens, TCRs are able to detect and respond to any peptide antigen that is presented on MHC. Furthermore, the TCRs can be restricted to MHC class 1 or MHC class 2, which can promote a more precise and relevant response with reduced levels of spurious or irrelevant activity. Accordingly, there is a wider availability of potential target antigens available to this approach that are relevant to stimulating a cellular response to nonself-antigens or aberrant cancer antigens.

The TCR can be MHC (or HLA) matched to the particular subject that may receive administration of the modified T cell. The TCR can be cloned from naturally occurring T cells or, alternatively, by a synthetic design. Techniques such as phage display can be utilized to develop TCRs that recognize the peptide antigen of interest. Implementation of transgenic TCRs can be accomplished, for example with viral (e.g., retroviral or lentiviral) vectors, as described in e.g., Baum et al., Molecular Therapy: The Journal of the American Society of Gene Therapy. 13:1050-1063 (2006); Frecha et al., Molecular Therapy: The Journal of the American Society of Gene Therapy. 18:1748-1757 (2010); and in Hackett et al., Molecular Therapy: The Journal of the American Society of Gene Therapy. 18:674-683 (2010); each of which is incorporated herein by reference in its entirety.

Cells

In another aspect, the disclosure also provides cells or cell compositions that are produced by the methods described herein. The composition is enriched for CD4+ and/or CD8+ cells or defined differentiated subset thereof, as described herein. The cells can be incorporated into therapeutic compositions appropriate for administration in adoptive cell therapy. The compositions comprise the cells or cell population and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, and the like. In some embodiments, the agents exist in the form of a salt.

The determination of appropriate carrier can be made based on the cells and/or the particular CAR or TCR that may be expressed by the cells, as well as the intended route of administration. The composition can further comprise one or more preservatives, such as selected from methylparaben, propylparaben, sodium benzoate, and benzalkonium chloride, and the like. The therapeutic composition can also comprise buffering agents. Exemplary, non-limiting buffering agents include citric acid, sodium citrate, phosphoric acid, potassium phosphate, and various other acids and salts.

The therapeutic composition comprises the cells at a sufficient amount that is appropriate and effective for the intended mode of administration and can be determined by persons of ordinary skill in the art.

Treatment Methods

In another aspect, the disclosure provides therapeutic methods that incorporate administration of the cells produced by the above methods or therapeutic compositions comprising the cells produced by the above methods. The cells or therapeutic compositions are administered to a subject in need thereof, e.g., a subject suffering from a disease or condition treatable by adoptive cell therapy. Exemplary diseases or conditions include cancers, infection by infectious agents (e.g., parasites, viruses, or bacteria), or allergic or immunodeficiency diseases.

The terms “subject” and “treat” are defined in more detail below in the “Additional definitions” section.

General and exemplary methods and techniques for administration of cells for adoptive cell therapy can be applied in the present aspect of the disclosure and are disclosed in more detail in e.g., in U.S. Pub. No. 2003/0170238; U.S. Pat. No. 4,690,915; Rosenberg, Nat Rev Clin Oneal. 8(10):577-85 (2011). See, e.g., Themeli et al., Nat Biotechnol. 31(10):928-933 (2013); Tsukahara et al., Biochem Biophys Res Commun 438(1):84-9 (2013); Davila et al., PLoS ONE 8(4):e61338 (2013); each of which is incorporated herein by reference in its entirety.

In some embodiments, the adoptive cell therapy is autologous, meaning that the subject receiving administration of cells produced by the presently disclosed methods is the same individual that was the source of the initial cells on which the culture methods were performed. In other embodiments, the adoptive cell therapy is allogenic, meaning the subject receiving administration of cells produced by the presently disclosed methods is a different individual from the source of the initial cells on which the culture methods were performed.

The present method can be applied to an individual with cancer or a neoplastic condition. While adoptive cell therapies, including administration of CAR T cells, to enhance the subject's response to cancers and infectious agents have advanced greatly in recent years, major technical hurdles remain. In many current approaches, the engineered cells exhibit a very potent activation after the initial exposure to the disease marker or antigen, but thereafter exhibit exhaustion. Exhaustion is characterized by a highly reduced proliferation and effector function. Thus, the therapy is often short lived allowing the transformed cell populations (e.g., tumors) to rebound. The cells produced from the disclosed methods to induce Notch signaling have resulted in prolonged state in the less differentiated subtype. This is shown to lead to prolonged longevity and persistence in the body with greater proliferative capacity and, ultimately, greater impact on tumor burden.

Furthermore, adoptive cell therapies such as administration of typical CAR T cells have faced particular challenges in effectively addressing solid tumors. Tumors often present hostile environments for immune cells, including the production of immunosuppressive signals that, inter alia, inhibit effector and proliferative capabilities of the immune cells. Furthermore, the concentration of antigens in a single locale can lead quickly to T cell exhaustion, which reinforces the loss of effector and proliferative function. While much research is currently applied to further manipulating administered cells, such as inhibiting checkpoint inhibitors, solid tumors remain difficult to treat with traditional adoptive cell therapies.

As described below, the cells produced by the disclosed methods, i.e., with induced Notch signaling as described above, are demonstrated to retain a less differentiated state for prolonged periods, but without inhibiting the capacity for proliferation. This results in extended persistence in vivo while maintaining a high proliferative capacity. Furthermore, the Notch-induced T cells exhibited a markedly lower susceptibility to exhaustion in the context of repeated or prolonged antigen exposure. Finally, it was demonstrated that tumor burdens are markedly reduced and survival of diseased individuals is significantly extended when treated with CAR T cells produced with Notch induction as compared to CAR T cells without Notch induction.

Accordingly, the present methods of administration and treatment encompass treatment of all types of cancers, including liquid tumors (e.g., hematological cancers, such as lymphoma (e.g., B cell malignancies), leukemia, and myeloma) and solid tumors. Solid tumors can be from any tissue of origin. Illustrative, non-limiting examples of solid tumors encompassed by these methods include glioblastoma, glioma, neuroblastoma, head and neck cancers, breast cancer, lung cancer (e.g., non-small cell lung cancer, lung squamous cell carcinoma), liver cancer, pancreatic cancer, mesothelioma, melanoma, prostate cancer, testicular cancer, osteocarcinoma, colorectal carcinoma, renal cell carcinoma, and ovarian carcinoma. Additional cancer targets, including solid tumor targets encompassed by the present application are described in more detail, including representative antigens appropriate for targeting, in, e.g., D'Aloia, M. M., et al., Cell Death and Disease, 9:282 (2018); Yeku, O., et al., Am Soc Clin Oncol Educ Book, 37:193-204 (2017); and Garber K, Nature Biotechnology, 36(3):215-219 (2018); each of which is incorporated herein by reference in its entirety.

As will be appreciated by persons of ordinary skill in the art, the cells can be appropriately designed and optimized to address the particular cancer of choice by engineering the immune receptor to the appropriate cancer antigen. Cells for adoptive T cell therapy (e.g., CAR T cells and TCR-expressing cells) encompassed by this method can be engineered to bind cell surface markers that distinguish any of these cancers. Ideally, the marker is unique to the target cancer, but is not necessarily so. TCRs encompassed by the present methods can be designed to a larger array of specific peptides characteristic of the cancer, including intracellular antigens that may not be recognized by the CAR T approach.

Administration of the cells occurs typically in the form of a therapeutic composition that also comprises carriers, excipients, optional buffering agents, and the like, appropriately formulated for the dose and mode of administration. Cells in adoptive cell therapy can be administered systematically, e.g., via I.V. injection, or locally to the site of infection or tumor.

As described above, the cells to be administered can comprise CD4+ T cells, CD8+ T cells, or CD4+ T cells and CD8+ T cells in any ratio. In some embodiments, the CD4+ T cells have been exposed to Notch receptor agonist and then subsequently mixed with, or administered coordinately with, CD8+ T cells that may or may not have been separately exposed to Notch receptor agonist.

Additional Definitions

As used herein, the term “nucleic acid” refers to any polymer molecule that comprises multiple nucleotide subunits (i.e., a polynucleotide). Nucleic acids encompassed by the present disclosure can include deoxyribonucleotide polymer (DNA), ribonucleotide polymer (RNA), cDNA or a synthetic nucleic acid known in the art.

In some embodiments, the Notch receptor agonist is an affinity reagent that has a specific binding affinity for Notch and, upon binding, induces Notch signaling with the cell. In some embodiments, the indicated affinity reagent is an antibody. As used herein, the term “antibody” encompasses antibodies and antigen binding antibody fragments thereof, derived from any antibody-producing mammal (e.g., mouse, rat, rabbit, and primate including human), that specifically bind to an antigen of interest (e.g., Notch). Exemplary antibodies include multi-specific antibodies (e.g., bispecific antibodies); humanized antibodies; murine antibodies; chimeric, mouse-human, mouse-primate, primate-human monoclonal antibodies; and anti-idiotype antibodies. The antigen-binding molecule can be any intact antibody molecule or fragment thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagent does not have to be naturally occurring or naturally derived, but can be further modified to, e.g., reduce the size of the domain or modify affinity for the Notch receptor as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phophodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/Notch binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with disease or condition (e.g., a cancer, infectious disease, or autoimmune disease). The term “therapeutic effect” refers to the reduction, elimination, or prevention of the disease or condition, symptoms of the disease or condition, or side effects of the disease or condition in the subject.

As used herein, characterization of a cell or population of cells being “positive” (or “+”) for a particular marker refers to the cell or population of cells having the detectable presence of the marker. Often, the marker is present or expressed on the surface of the cell. The marker can be detected using any conventional techniques. To detect the surface expression, for example, the marker can be detected using immune-staining based techniques. For example, an antibody specific for the marker can be exposed to the cell or population of cells and the binding of the antibody can be imaged or detected by flow cytometry. Conversely, use of the term “negative” (or “−”) refers to the absence of a substantial presence in or on the surface of the cell.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

Title: Effect of Notch Signaling on T Cell Development Ex Vivo.

Introduction: As described in more detail above, the role of Notch signaling in differentiation of cell types from HSC to mature T_{E} cells is unclear. The present study aims to clarify the role of Notch signaling for potential influence on the differentiation of T cell subtypes, and to address the potential practical application of this role in cultivating and expanding T cells for adoptive cell therapy.

Results and Discussion:

Initial assays adapted from Delaney, et al., Blood, 106(8): 2693-2699 (2005) (incorporated herein by reference in its entirety) were performed to determine the effect of Notch signaling ex vivo of cells. Briefly, naïve T cells were cultured for 4 hours on plates coated with Retronectin and different concentrations of immobilized DLL1^{Ext IgG} (a Notch agonist) or IgG1 (as control), or were cultured in simple tissue culture (TC) without any immobilized agonist or other moiety. The RNA was isolated and then transcribed into cDNA. Hes1 expression was measured by SYBRgreen q-PCR. This culture method results in Hes1 upregulation, demonstrating Notch signaling. The assay design considers that cell-cell interactions and physical “pulling” or tension applied in the Notch-receptor and ligand interaction is important for signaling. Thus, the ligand is coated on a plate with Rectronectin, which effectively immobilizes the ligand to the plate surface. As illustrated in FIG. 1, dose-dependent Hes1 upregulation occurred in T cells after exposure to immobilized DLL1, showing Notch signaling. The same effect was not observed in the IgG or TC controls. It was noted that low cell density was observed as being important for assay accuracy because it limits cell to cell signaling that could influence the response, which is independent of coated ligand presence or density.

To ascertain the effect of Notch signaling on the phenotype and differentiation state of the T cells, including expression of Notch receptors and ligands, the above assay was expanded. Specifically, at D −1, the tissue culture plates were coated overnight with the Rectronectin and ligand of choice (i.e., immobilized DLL1 or IgG1 control). At D0, 2×10⁵ naïve T cells were plated with anti-CD3/CD28 DYNABEADS® (ThermoFisher) added (e.g., at 3:1 ratio) to stimulate growth and proliferation. Cells were removed at intervals (e.g., every 24 hours) and RNA was isolated and sequenced for quantification of Hes and other gene expression levels. In most assays, gene expression was quantified using Q-PCR using Taqman gene expression assays. A more detailed description of these exemplary methods is set forth below in Example 5.

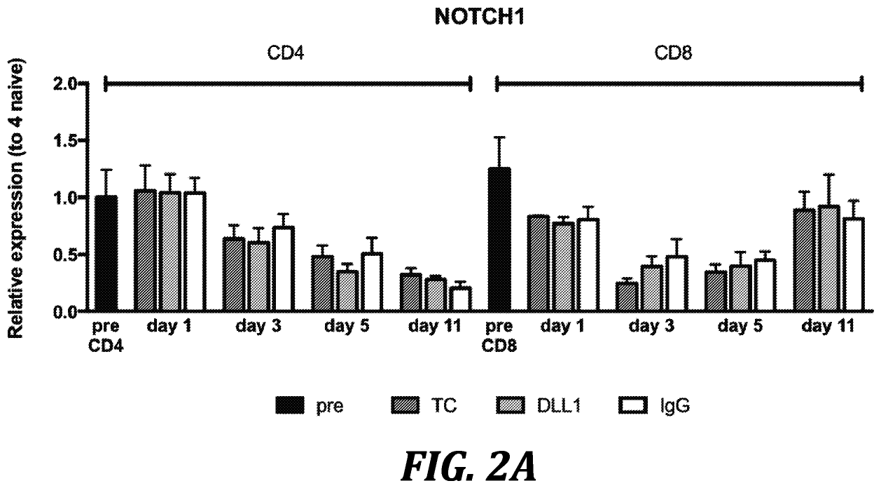

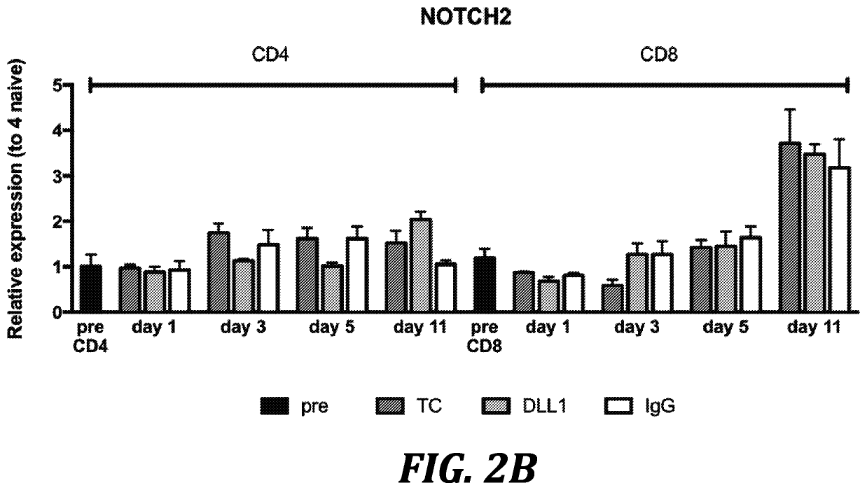

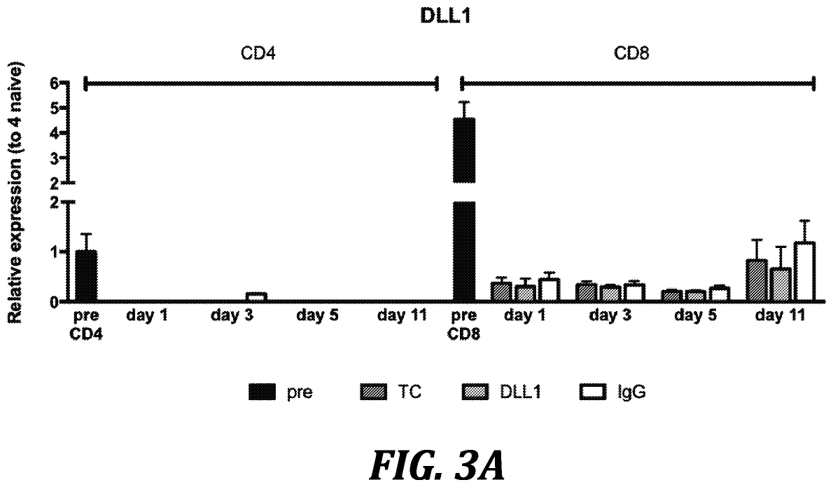

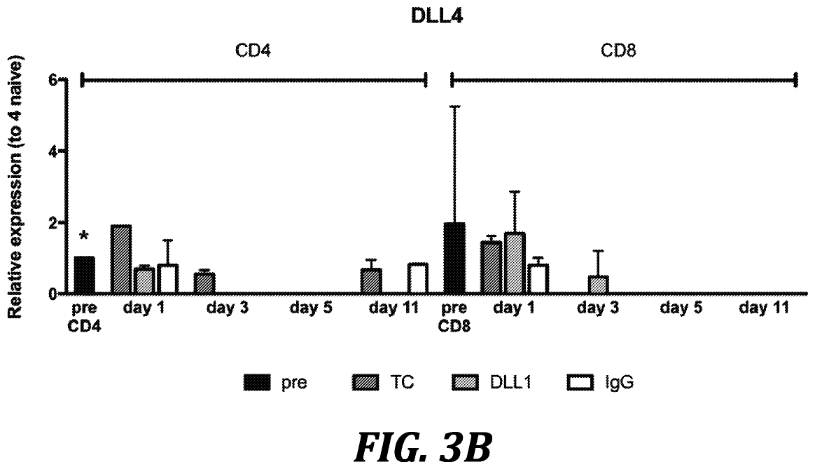

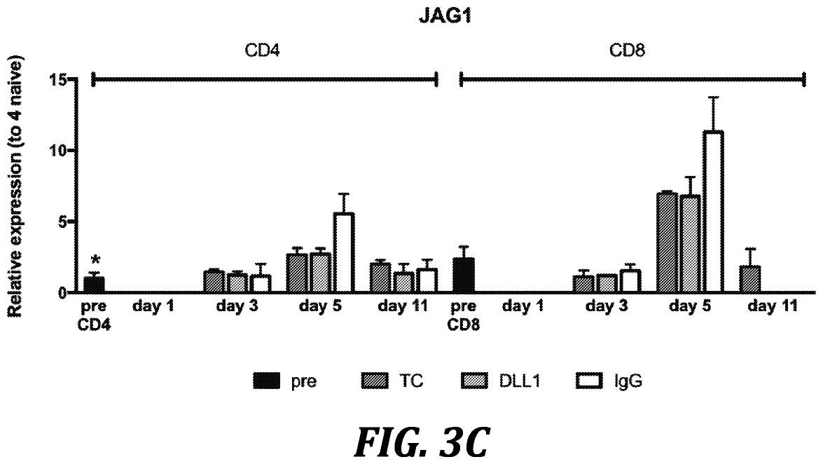

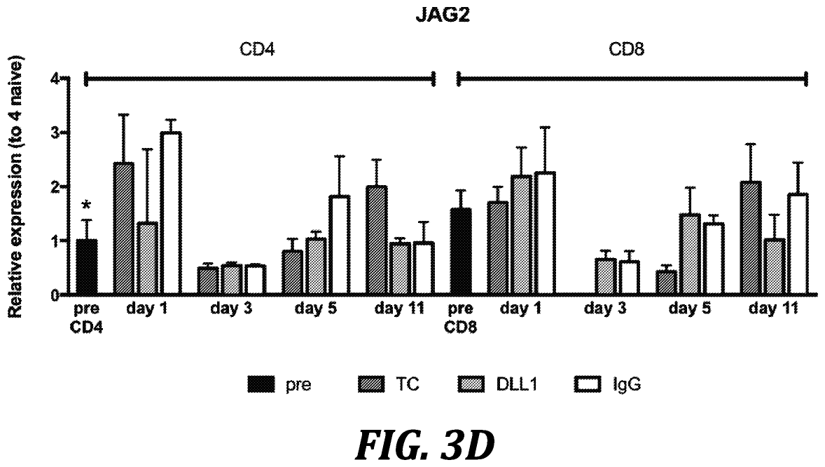

Naïve T cells that were cultured according to this assay design were assessed for relative expression of Notch receptors, Notch ligands, and other cell surface markers. FIGS. 2A and 2B graphically illustrate the relative expression of Notch1 and Notch2 receptors, respectively, in human CD4+ and CD8+ T cells at different time points before and after culture in different conditions. The cultures are indicated in the lower legend (TC=Tissue culture only; DLL1=immobilized DLL1 Notch agonist (experimental condition); IgG=immobilized IgG (control ligand)). As shown, Notch 1 and 2 receptors are not differentially expressed between the different ex vivo culture conditions. Notch1 expression generally declines during culture in both CD4+ and CD8+ T cells, although there is an increase at day 11 in CD8+ cells. Notch 2 stays the same in CD4+ T cells, but increases on day 11 in CD8+ T cells. FIGS. 3A-3D graphically illustrates the relative expression of Notch ligands DLL1, DLL4, JAG1, and JAG2, respectively, in the ex vivo cultivated CD4+ and CD8+ T cells in the different conditions. The cultures are indicated in the lower legend, (TC=Tissue culture control, DLL1=the Notch agonist for experimental condition, and IgG is the control ligand). As shown, JAG1 and JAG2 agonists had some variable expression between conditions. JAG1 generally exhibited an initial increase in expression in both CD4+ and CD8+ cells in all culture conditions, follow by a decrease after day 5. JAG2 exhibited an initial reduction of expression after culture, followed by a general increase up to day 11. DLL1 and DLL4 ligands could only be detected at low levels at most time points after exposure to the experimental culture conditions.

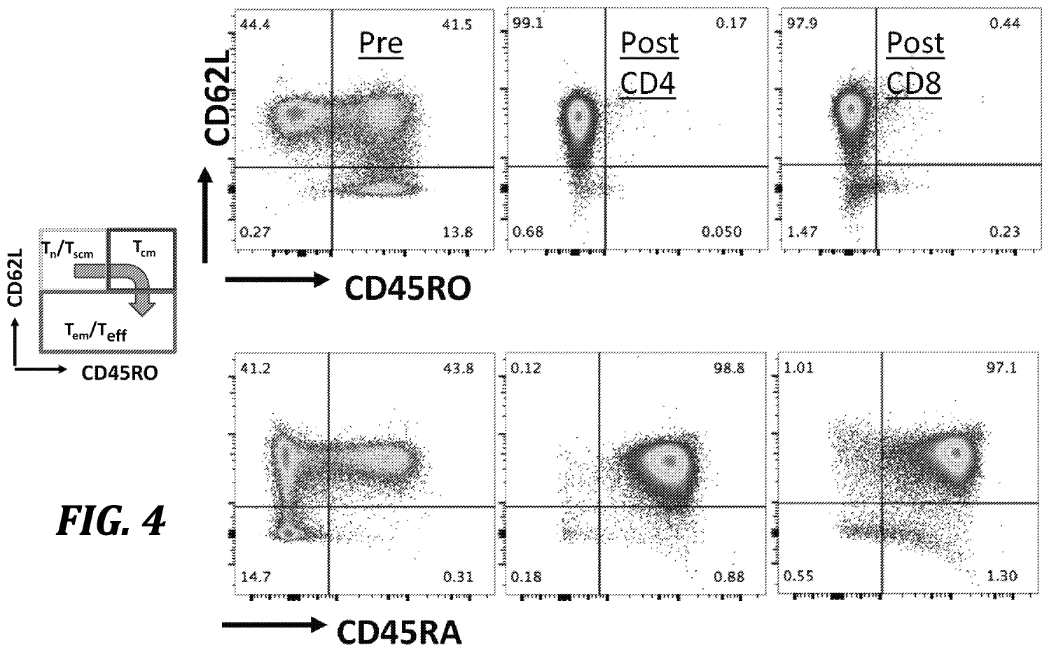

To assess the effect of Notch on the development and efficacy of genetically modified immune cells, e.g., CAR T cells, human naïve T cells cultured in the presence of Notch1 agonist, or control ligand, or TC control, as described above, were further engineered by transduction at day 1 with CD19-specific CAR to create human CD19-CAR T cells ex vivo. The cells were split and media was changed every 2-3 days. The engineered CAR T cells were removed at day 5 to perform counts and assess phenotype. All cells were eventually transferred to tissue culture only plates and assessed at day 11 for phenotype and functionality. Phenotype was assessed using flow cytometry with anti-CD62L, anti-CD45RO, and anti-CDRA antibodies and a gating strategy as illustrated in FIG. 4 to differentiate between T_{N}/T_{SCM}, T_{CM}, and T_{EM}/T_{E} cells.

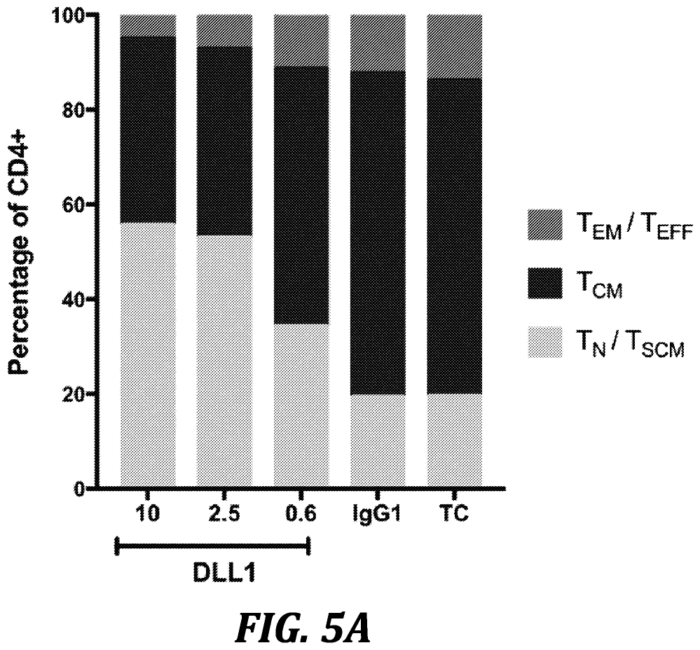

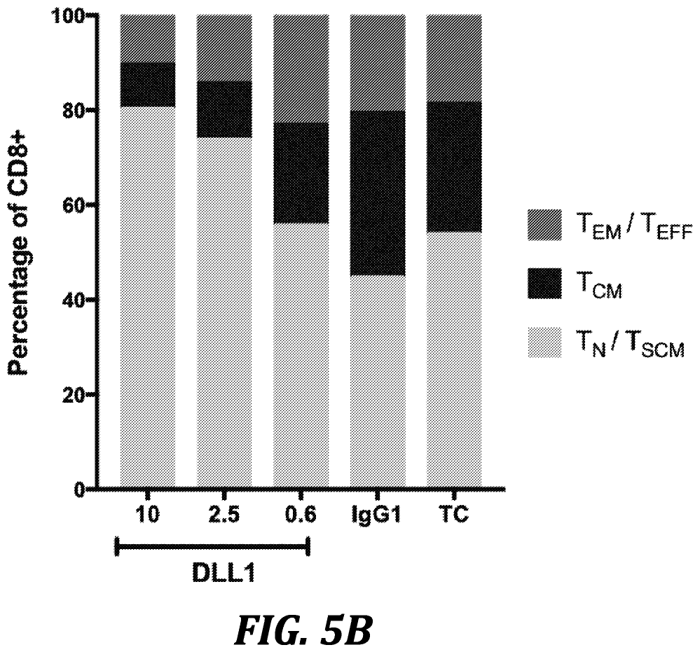

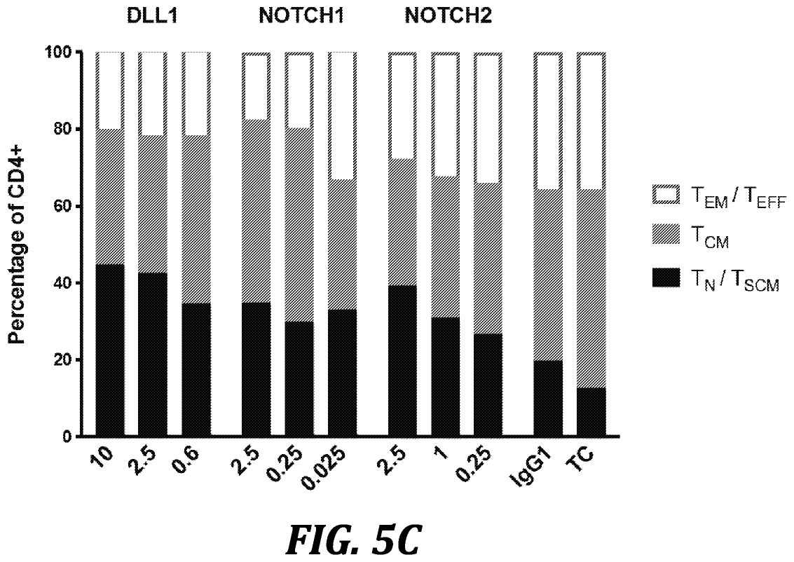

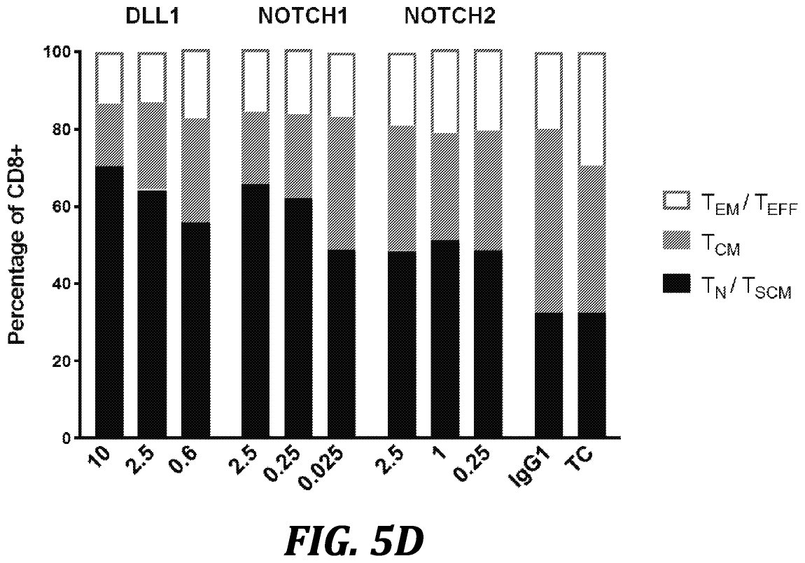

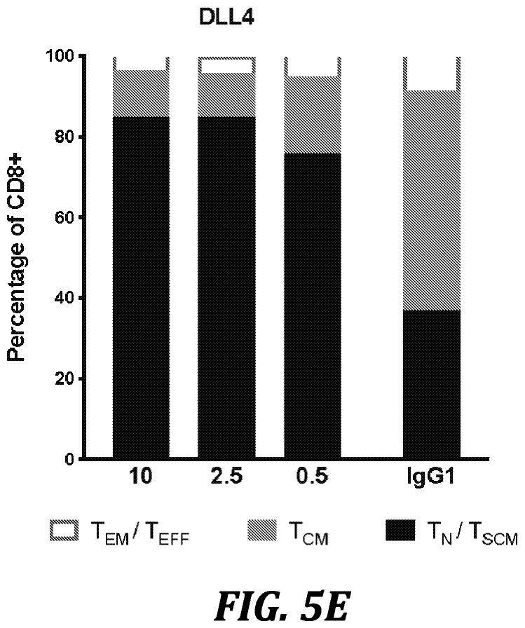

FIGS. 5A-5E illustrate results of flow-based assays to determine the differentiation phenotype of T cells exposed to different Notch agonists versus control ligand and TC control, as described above. Prior to analysis, human naïve T cells were stimulated with anti-CD3/CD28 DYNABEADS® (ThermoFisher)and incubated with the indicated Notch ligand, IgG1 (as an irrelevant ligand control), or in simple tissue culture (TC; as a no ligand control) in media containing IL-2.

FIGS. 5A and 5B graphically illustrate the proportion of T cell subtypes after culture with different doses of DLL1 agonist (and control conditions). As shown, culturing T_{N} on DLL1 plates and engineered to contain CD19-CAR increases the percentage of CD62L+CD45RO− CAR T cells (i.e., the T_{N}/T_{SCM} subset) for both CD4+ cells (FIG. 5A) and CD8+ cells (FIG. 5B), as compared to controls. This effect is dose dependent. The cells were also assessed for expression of a panel of cell surface markers, CD27, CD28, CD95, PD1, LAG3, and CD69 after 5 days of stimulation with anti-CD3/CD28 DYNABEADS® (ThermoFisher)for both the CD4+ T cells and CD8+ T cells. This demonstrates that cultures exposing CAR T_{N} cells to Notch agonist DLL1 results in cells having lower expression of activation markers PD-1, LAG3 and CD69. For CD4+ T cells, DLL1 culturing increased CD27 and CD28 expression, but lower CD95 levels. For CD8+ T cells, expression of CD28 is increased. Other Notch receptor agonists similarly resulted in increased proportions of CD62L+CD45RO− CAR T cells (i.e., the T_{N}/T_{SCM} subset) both CD4+ cells and CD8+ cells, as compared to controls. FIGS. 5C and 5D graphically illustrate elevated proportions of CD62L+CD45RO− CAR T cells (i.e., the T_{N}/T_{SCM} subset) for CD4+ T cells and CD8+ T cells, respectively, after culturing with anti-Notch1 antibody (i.e. N1 antibody) or with anti-Notch2 antibody (i.e. N2 antibody) relative to controls. FIG. 5E graphically illustrate elevated proportions of CD62L+CD45RO− CAR T cells (i.e., the T_{N}/T_{SCM} subset) for CD8+ T cells, respectively, after culturing with Notch agonist DLL4 relative to controls. Accordingly, it is demonstrated that Notch signaling maintains an early developmental phenotype for longer in both CD4+ cells and CD8+ T cells.

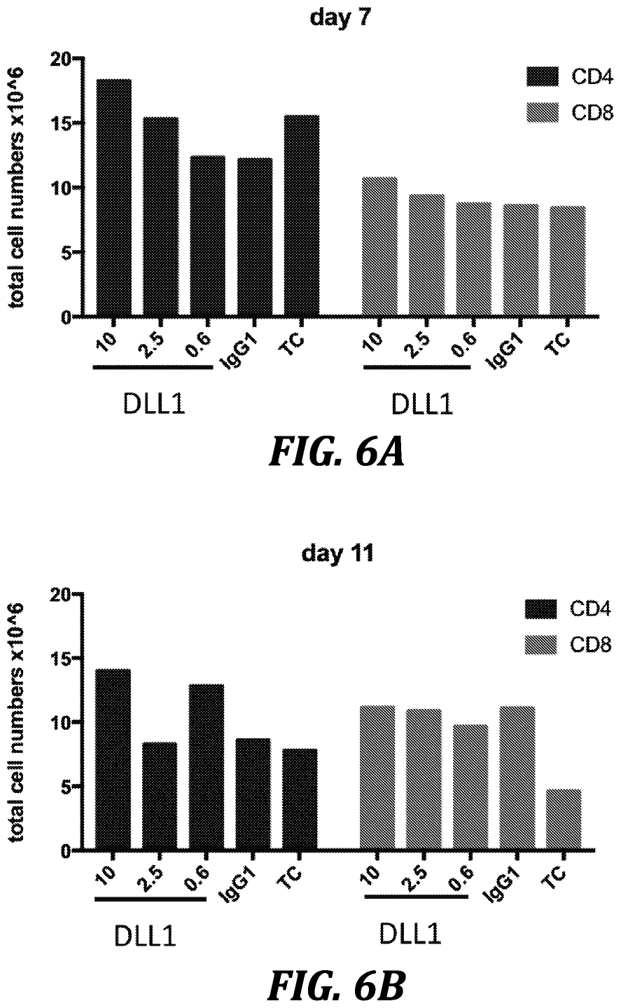

Total cell numbers were also quantified to ascertain whether Notch signaling affects CD4+ T cell and CD8+ T cell proliferation. FIGS. 6A and 6B graphically illustrate the total number of CD4+ T cells and CD8+ T cells measured at day 7 and day 11 in culture with different concentrations of immobilized DLL1, IgG1 control, or TC control. The cultures also included anti-CD3/CD28 DYNABEADS® (ThermoFisher), respectively. As illustrated, Notch signaling does not inhibit CD4+ T cell or CD8+ T cell proliferation. Furthermore, the data indicate that exposure to Notch ligand may even increase CD4+ T cell proliferation at higher concentrations.

Conclusion: These results indicate that culturing T cells in the presence of Notch agonist allow for quantitative signaling through the Notch pathway. Additionally, signaling through Notch during culture of naïve T cells resulted in a less differentiated phenotype, as determined through observations of CD45RO, CD62L, costimulatory molecules (i.e., CD27 and CD28), and activation markers (i.e., PD-1, LAG-3, and CD69). The promotion of less differentiated phenotypes was demonstrated with a variety of different Notch agonists including Notch ligands and anti-Notch antibodies. However, inducing Notch signaling did not inhibit proliferation and functionality, but rather may even promote proliferation.

### Example 2

Title: Performance of Notch-Induced CAR T Cells in Adoptive Cell Therapy In Vivo

Introduction: It is demonstrated in Example 1 that culturing “young” or relatively undifferentiated T cells ex vivo with a Notch agonist allows for quantitative Notch signaling. This resulted in less differentiated phenotypes but did not inhibit (and may even enhance) proliferation or functionality of the cells, including cells that were further engineered to express heterologous CAR. Next, CAR T cells generated from ex vivo cultures of initial T_{N} cells, as described above in Example 1, were assessed for performance in adoptive cell therapy in a Raji murine model.

Results and Discussion:

The Raji lymphoma model in immunodeficient Nod/Scid/gamma chain −/− (NSG) mice, generally described in Sommermeyer et al., Leukemia volume 30, pages 492-500 (2016), incorporated herein by reference in its entirety, was used to assay the persistence and anti-tumor effect of human CAR T cells cultured in the presence or absence of Notch receptor agonist, as described above, prior to administration in the subject mice.

Briefly, human CAR T cells were produced similar to the protocol generally described above. At day −1 the culture plates were prepared by coating 2.5 μg N1 antibody (an anti-Notch receptor agonist; LEAF Biolegend antibody) or IgG1 control ligand. At day 0, naïve T cells were plated at 2×10⁵ CD8+ per well in 24 wells with anti-CD3/CD28 DYNABEADS® (ThermoFisher) at a 3:1 ratio. At day 1, CD19-CAR transduction was performed. At D4, 7-8 week old NSG male mice were injected with 5×10⁵ Raji cells (labeled with GFP/ffluc). The CAR T cells were removed from culture at day 5 (via DYNABEADS® removal) and the cells were counted and assessed for phenotype by flow cytometry. At day 7, the cells were transferred to TC plates/flasks. The media was changed every 2-3 days throughout the culture. Finally, at day 11, the cells were counted and assessed for phenotype and functionality. The NSG mice previously injected with Raji cells were injected with 7×10⁵ EGFRt+ cells (2×10⁵ CD4+ and 5×10⁵ CD8+) CAR T cells cultured in one of the three conditions (i.e., with N1 antibody, IgG1, or TC control). The mice T cell populations were monitored by periodic bleeds. Tumor burden was assessed by bio-layer interferometry (BLI) and the mice were ultimately monitored for survival. More detail for exemplary methods and materials for the ex vivo culture is described below in Example 3.

To ensure all groups received equivalent effective doses of CAR T cells, transduction efficiency was checked prior to infusion. Flow cytometric analysis of pre-infusion T cells was performed after staining with EGFR-biotin followed by streptavidin-PE to identify the frequency of T cells expressing the CAR. The dose was optimized by analysis of pre-infusion flow assay data profiling CD4, CD8, and EGFR expression on lymphocytes (not shown) and determined to be 3×10⁶ total T cells, of which ˜6×10⁵ were EGFR+ with around 80% CD8+ T cells.

As indicated, the phenotypes of the cultured human CAR-expressing CD4+ T cells and CD8+ T cells were assessed at day 5 and day 11 using flow cytometry to characterize expression of a panel of surface markers on the cells. At day 5, N1-exposed CD4+ T cells expressed higher levels of CCR7, CD28, CD27 and CD62L, and lower levels of CD95, PD-1, Lag-3, CD25 and CD69, suggesting a less differentiated/activated phenotype. Similarly, N1-exposed CD8+ T cells express higher levels of CCR7, CD28, CD27 and CD62L, and lower levels of CD95, PD-1, Lag-3, CD25, CD69 and Tim-3, also suggesting a less differentiated/activated phenotype.

At day 11 of culturing before infusion, expression of CD45RO, CD45RA and CD62L was determined for CD8+ T cells cultured on 2.5 μg N1 antibody (“N1-exposed”), IgG or TC control cells, untransduced cells (mock) and compared to staining of a PBMC sample. The N1-exposed T cells show higher percentages of CD62L+ cells, higher expression of CD45RA and lower expression of CD45RO, indicating a less differentiated phenotype. Furthermore, expression of CD95, CCR7, CD28, CD27 and CD62L was determined for the CD8+ and CD4+ T cells cultured on 2.5 μg N1 antibody, IgG or TC control cells and untransduced cells (mock) and compared to staining of a PBMC sample. At day 11 most markers were similar for all conditions, except CCR7 is lower in the IgG condition for CD8+ and CD4+. Expression of additional cell surface markers PD-1, LAG-3, CD25, CD69 and Tim-3 at day 11 pre-infusion was determined for the CD8+ and CD4+ T cells cultured on 2.5 μg N1 antibody, IgG, or TC control cells and untransduced cells (mock), and compared to staining of a PBMC sample. Most markers were similar for all conditions, except CD25 was lower in N1-exposed T cells. This difference was slight for CD8+ T cells, but was more profound for CD4+ T cells.

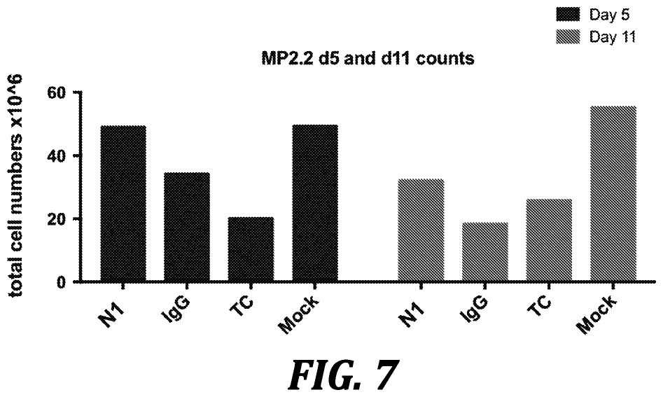

To assess whether Notch signaling using the 2.5 μg N1 anti-Notch receptor agonist antibody culture conditions influences T cell proliferation, the total cell numbers from the cultures were quantified at pre-infusion times. FIG. 7 graphically illustrates the total T cell numbers (CD4+ and CD8+) at day 5 and day 11 for cells incubated with N1 antibody, as compared to IgG1 as negative control or tissue culture (TC) only. This indicates that the less differentiated phenotype is not a result of limiting proliferation in this system. A carboxyfluorescein succinimidyl ester (CFSE) proliferation assay was also conducted to assess proliferation of the N1-exposed CAR T cells after infusion into mice with relevant CAR targets. The CFSE assay measures the proliferation of CAR T cells cultured with CD19+ tumor cells (K562/CD19 or Raji) or in media alone. Cell proliferation is measured by the dilution of CFSE. This assay demonstrated that N1-exposed T cells proliferate against CD19-positive targets (K562-CD19 and Raji), for both CD4+ and CD8+ T cells (not shown).

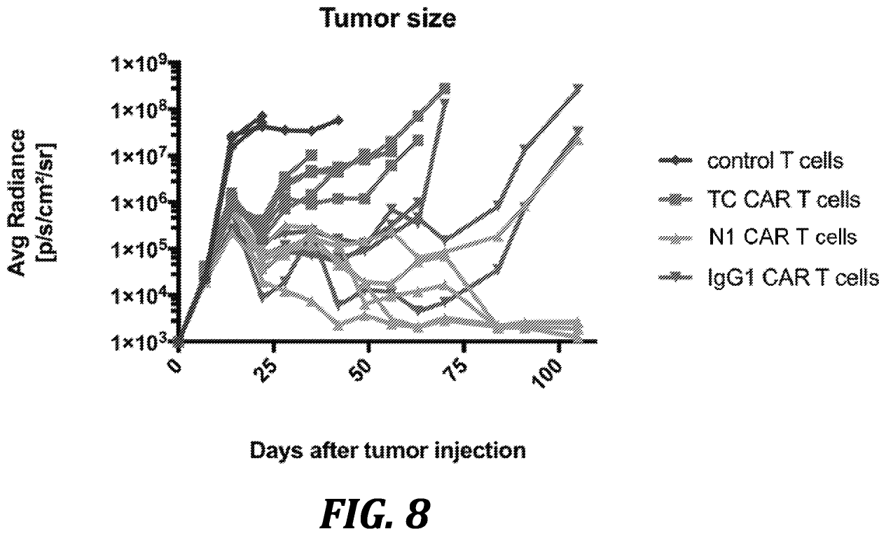

To assess functional performance of the N1-exposed CAR T cells on tumor targets, tumor burden was monitored in mice (previously engrafted with Raji cells expressing firefly luciferase) by bioluminescence imaging over extended periods after administration of the N1-exposed CAR T cells, or with TC or IgG1 control cultures. Photographs of mice were developed showing tumor burden indicated heatmap overlays. At day 29, two of the mice in the Control T cell group (no CAR expression) had already been euthanized due to tumor burden. Four out of five mice treated with N1-exposed CAR T cells were tumor-free by day 84, while both remaining mice in the IgG group had tumors and all mice in the TC group had been euthanized. FIG. 8 graphically illustrates the tumor burden of individuals administered with control T cells, tissue culture (TC) CAR T cells, Notch-1 stimulated CAR T cells, and IgG1 CAR T cells, as indicated by bioluminescence imaging (BLI). All time points are after injection of mice with Raji tumor cells. Each line representing a mouse and symbols depict individual data points. As illustrated, the tumor burdens in the mice receiving N1-exposed CAR T cells were greatly reduced and generally continued to decline over the 100 days post infusion. These data illustrate a remarkable efficacy of the N1-exposed CAR T cells to induce effective responses against tumors in vivo. Moreover, the efficacy is maintained over prolonged periods, indicating long-term persistence of the CAR T cells cultured in conditions inducing Notch signaling.

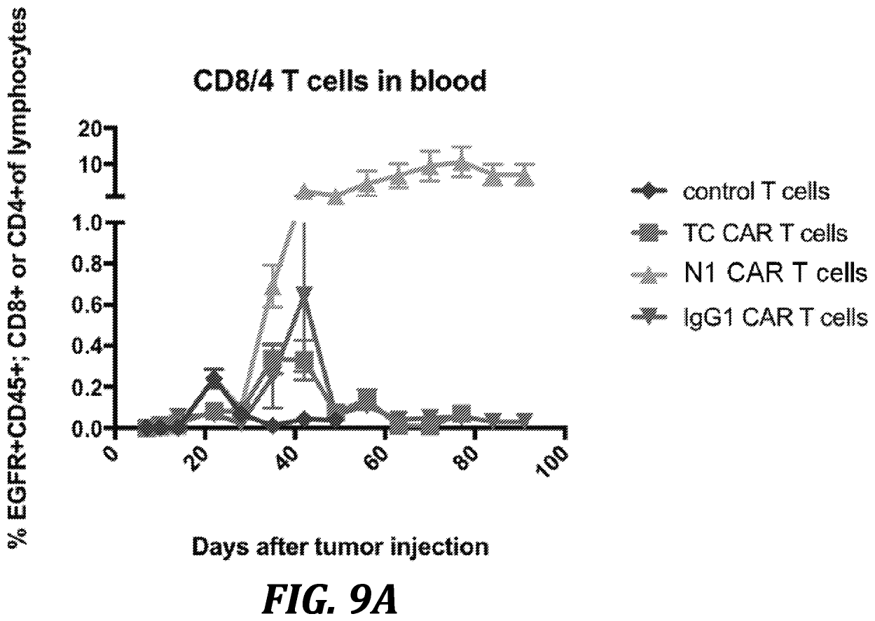

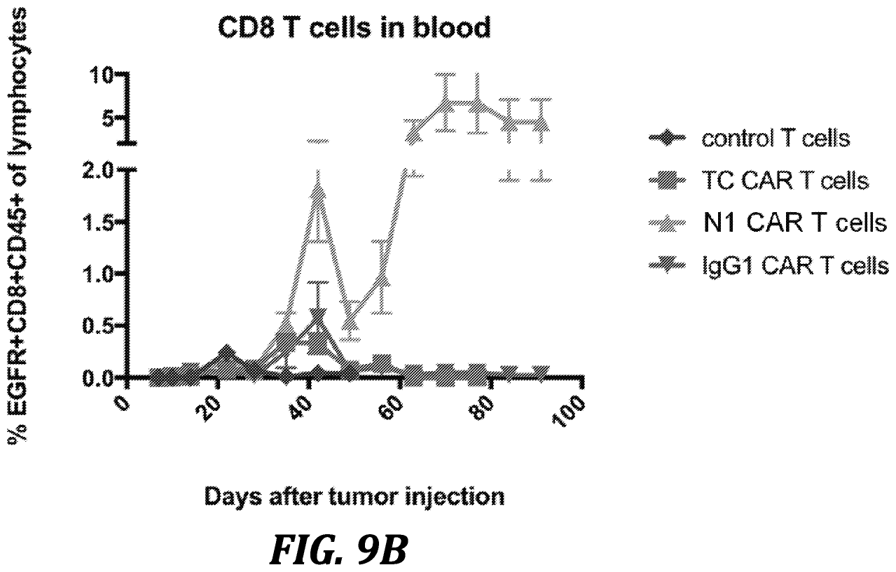

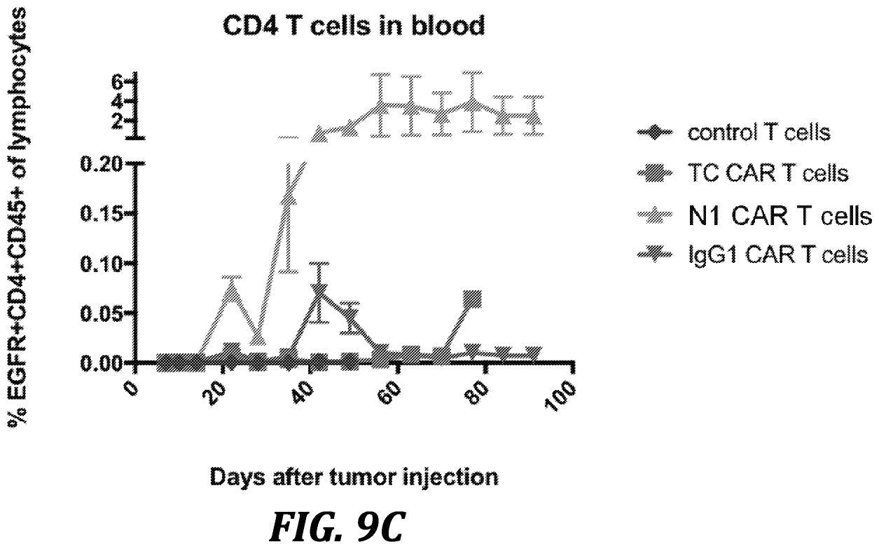

The T cells appearing in the blood of Raji-engrafted mice were assessed at several time points post-infusion and Raji tumor injection. As described above, the Raji-engrafted mice were treated with control T cells, TC CAR T cells, N1-exposed CAR T cells (exposed to Notch1 agonist antibody during the generation of the CAR T cells), and IgG1-exposed CAR T cells. Blood was lysed with ammonium chloride potassium solution, and then stained with antibodies for CD45, CD4, CD8 and EGFR. Data was collected on a Canto II flow cytometer. FIGS. 9A-9C graphically illustrate the levels of T cells in the blood at the multiple time points, with FIG. 9A illustrating the levels of CD8+ and CD4+ T cells, FIG. 9B illustrating the levels of CD8+ T cells, and FIG. 9C illustrating the levels of CD4+ T cells. As shown, Raji mice treated with N1-exposed T cells have much higher frequencies of EGFR+ CD4+ and CD8+ T cells than control groups, from day 35 on. It is noted that tumor burden is already lower at the day 35 timepoint, further indicating prolonged survival and persistence of N1-exposed T cells resulting in enhanced anti-tumor activity over a prolonged period of time.

Cytometric analyses were performed on T cells collected from Raji-engrafted mice at several timepoints after infusion. On day 56 and 91, the analyses were gated for CD45+ cells and staining was performed for CD4+ and CD8+ versus EGFR (not shown). These results demonstrated that mice treated with N1-exposed CAR T cells had higher frequencies of EGFR+ and EGFR− cells in their blood, indicating that Notch1 signaling results in T cells with higher proliferative capacity and persistence, independent of the cells' exposure to antigen in vivo.

To determine whether the effects observed above are due to CAR T cells infused into the mice or GvHD from untransduced T cells (that are EGFR−) and which have been co-infused, the assay was repeated with an additional EGFR sorting prior to infusion to the mice. Like the assay described above, naïve human T cells are incubated with Notch1 agonist antibody, control (IgG1), or in control conditions without IgG1 (TC), transduced with CD 19 CAR and eventually transfused into mice already engrafted with Raji lymphoma cells. However, before adoptive transfer of the T cells, EGFR+ cells were sorted to address whether the Notch1 effect previously observed is due to CAR T cells or to any allo-reactivity by EGFR− cells. Specifically, the cells were stained with EGFR-biotin, followed by streptavidin-PE to allow the EGFR+ cells. The CAR transduction efficiency was similar for the different groups (not shown). Expression of surface markers on the transduced T cultured cells was also assessed by flow cytometry to ascertain the phenotype of the transduced T cultured cells. At day 5 (pre-infusion), both N1-exposed CD4+ T cells and N1-exposed CD8+ T cells show higher percentages of CD62L+ cells, higher expression of CD45RA and lower expression of CD45RO. These data indicate a less differentiated phenotype.

In a similar assay, additional surface markers were assessed at day 5 (pre-infusion) of the CD4+ and CD8+ CART cells. Specifically, expression of CD95, CCR7, CD28, CD27, CD62L, as well as PD-1, Lag-3, CD25, CD69 and Tim-3 was determined. For both the CD4+ CAR T cells and the CD8+ CAR T cells, the phenotypes had differences between the culturing conditions, with N1-exposed T cells expressing higher levels of CCR7, CD28, CD27 and CD62L, and lower levels of Lag-3 and CD69 compared to the IgG1-exposed or TC control cells, ultimately suggesting a less differentiated/activated phenotype.

To address a later time point, expression of surface markers was assessed again by flow cytometry at day 11 (pre-infusion) on the transduced T cells to ascertain the phenotype of the transduced T cultured cells. Both N1-exposed CD4+ T cells and N1-exposed CD8+ T cells continued to exhibit higher percentages of CD62L+ cells, higher expression of CD45RA and lower expression of CD45RO compared T cells incubated with IgG1 control or in TC. This indicates the continued and prolonged maintenance of a less differentiated phenotype during ex vivo culture after with Notch signaling by the N1 anti-Notch receptor agonist antibody.

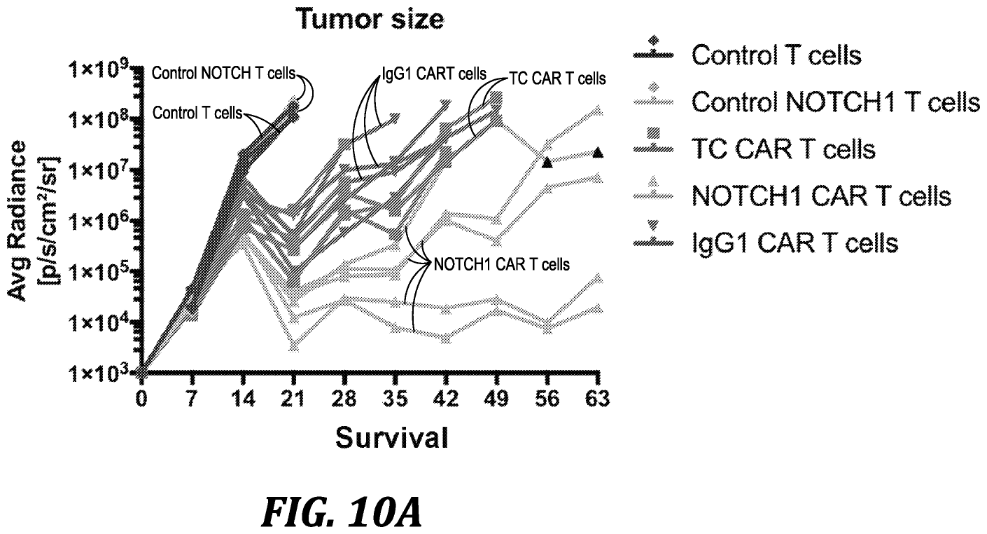

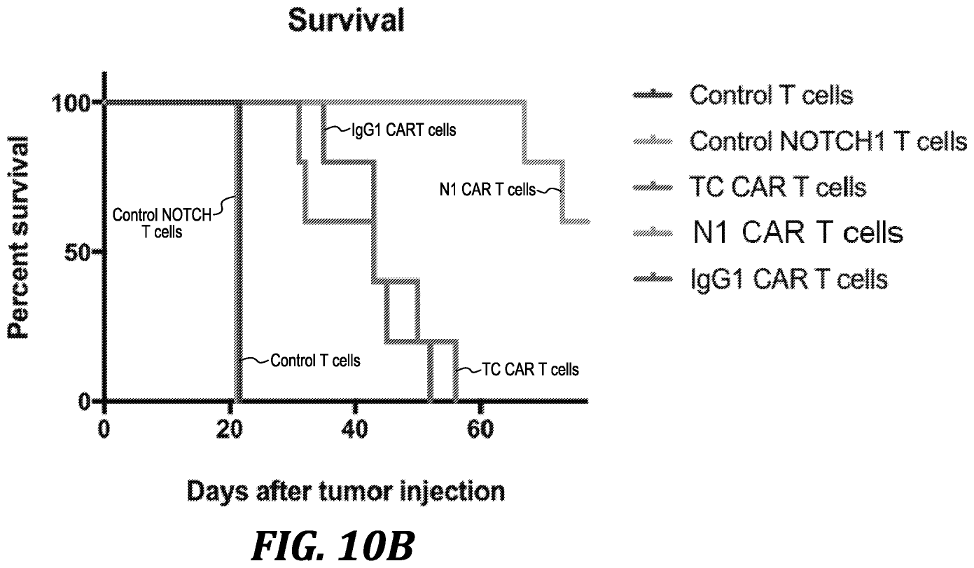

To assess functional performance of these N1-exposed CAR T cells (sorted for EGFR+) on tumor targets, tumor burden was monitored in Raji-engrafted mice by bioluminescence imaging (BLI) as described above. The Raji cells expressed firefly luciferase for imaging. For comparison, the mice were administered CAR T cells previously exposed to N1 anti-Notch receptor agonist, IgG1 control, or TC (no IgG1 control). Images of mice were developed with tumor burden indicated in heatmap overlays. By day 21, a clear increase in antitumor activity was observed in the CD19 CAR N1 group compared to the controls. FIG. 10A graphically illustrates the tumor sizes, as indicated by bioluminescent radiance, at times after engraftment of mice with Raji cells, with each line representing a mouse, and symbols depict individual data points. As illustrated, the tumor sizes in the mice receiving N1-exposed CAR T cells were greatly reduced compared to control CAR T cells. FIG. 10B illustrates survival curves of the Raji-engrafted mice. As shown, individuals transfused with the N1-exposed CAR T cells had significantly extended survival with more than half of the subject remaining alive by the end of the study. In contrast, all Raji-engrafted mice receiving equivalent doses of control T cells (no CAR expression or N1 exposure) or control N1-exposed T cells (no CAR expression) died by day 20 after engraftment. Raji-engrafted mice receiving TC control CAR T cells (no N1 exposure) or IgG1-exposed CAR T cells had extended survival times but all of the mice in each group eventually died between day 50 and 60 post-engraftment. This illustrates a remarkable efficacy over prolonged periods of the N1-exposed CAR T cells to induce effective responses against tumors in vivo. Furthermore, this data confirms that the performance of the adoptive cell therapy, as described above, is due to the Notch-stimulated (e.g., N1-exposed) CAR T cells themselves and not allo-reactivity of any EGFR− cells.

Conclusion: These results clearly demonstrate that CAR T cells cultured in the presence of Notch agonists retain a relatively undifferentiated state for prolonged periods of time. Upon administration to subjects with tumors, the administered CAR T cells exhibit proliferation upon exposure to the relevant target as well as prolonged survival and persistence within the subject, demonstrating a prolonged resistance to exhaustion. Practically, this resulted in significantly reduced tumor size over prolonged periods. Finally, these effects were determined to be the result of the CAR T cells cultured in the presence of Notch ligands, and not due to allo-reactivity of any non-transduced T cells.

### Example 3

Title: Persistence of Notch-Induced CAR T Cells Exposed to Prolonged Presence of Antigen.

Introduction: A major obstacle to therapeutic applications of CAR T cells in anti-viral and anti-cancer settings is their exhaustion, i.e., limited temporal effector efficacy, during prolonged or chronic exposure to target antigen. Exhausted CAR T cells are typically found to have low proliferative capacity, low production of cytokines, express high levels of inhibitory surface receptors, and experience high rates of apoptosis. These exhaustion traits severely limit efficacy of the adoptive CAR T therapeutic approach. Recent work has indicated that CAR structure, and especially the structure of the CAR ectodomains, can critically impact functionality and exhaustion of the CAR expressing T cells. See, e.g., Long, A. H., et al., Nat. Med., 21(6):581-590 (2015), incorporated herein by reference in its entirety.

It is demonstrated above that CAR T cells that have been generated in the presence of Notch agonist results in a less differentiated phenotype without inhibition of proliferation and functionality. Additionally, it is demonstrated above that administration of these CART cells generated in the presence of Notch agonist have enhanced efficacy and persistence in vivo as compared to control CAR T cells that were not exposed to a Notch agonist. It was demonstrated in NSG mice with engrafted Raji lymphoma that adoptive transfer of the CAR T cells with induced Notch signaling resulted in significantly reduced tumor burden and enhanced survival over prolonged periods compared to adoptive transfer of control T cells, and control CAR T cells that were not exposed to Notch agonist.

To specifically assess persistence characteristics and susceptibility to exhaustion, the CAR T cells exposed to Notch agonist were provided prolonged exposure to antigen conditions.

Results and Discussion:

As described above in more detail, naïve T cells were cultured with Notch agonist or control conditions (IgG1 antigen or no antigen TC control). The culture medium also contained anti-CD3/CD28 DYNABEADS® (ThermoFisher) at a 3:1 ratio. At day 1 the T cells were transduced to express CD19-specific CAR. The transduction efficiency and consistency among the culture groups was confirmed. Unlike the assay format described above in Example 1, the target antigen was added to the CAR T cell culture multiple times in the course of the assay to replicate chronic antigen exposure. Specifically, in two different trials, K562-CD19 cells or Raji lymphoma cells were added to the CAR T cell cultures at day 2, day 4, and day 7 at a 1:1 effector:target ratio in a 96-well plate format. The target cells were not irradiated and the medium lacked IL-2. The cultures were assessed by flow cytometry to determine normalized T cell numbers during the course of the assay.

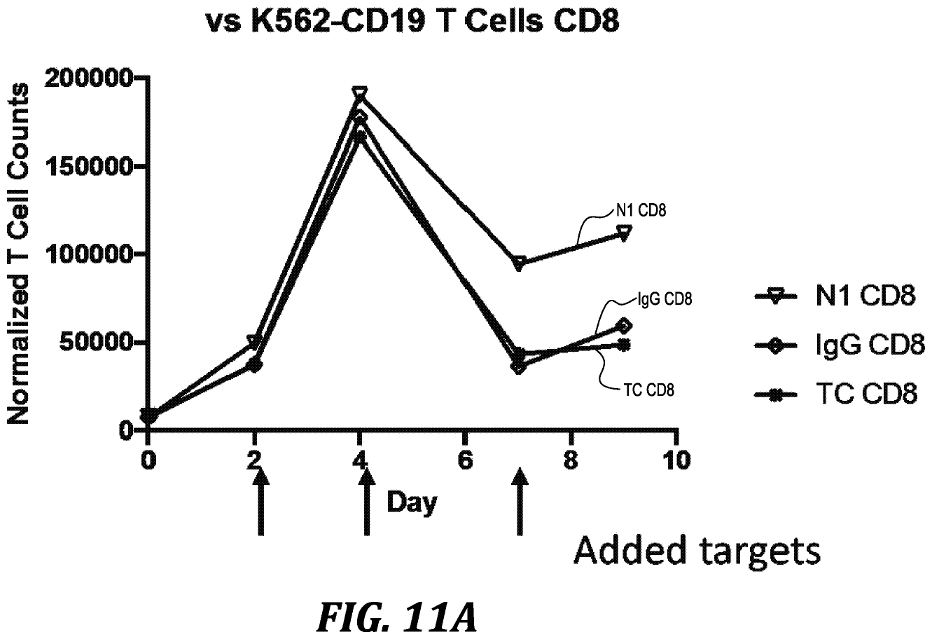

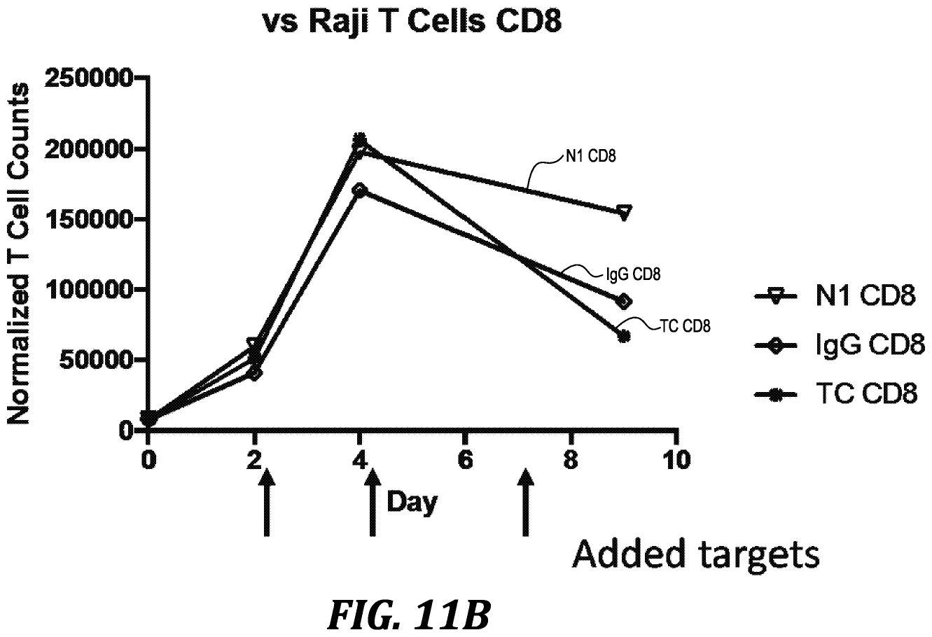

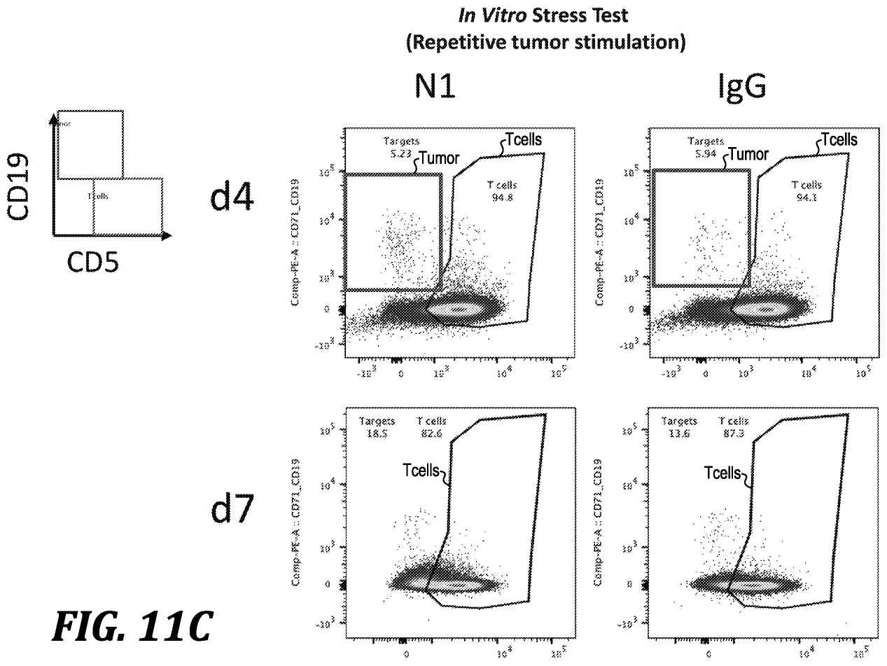

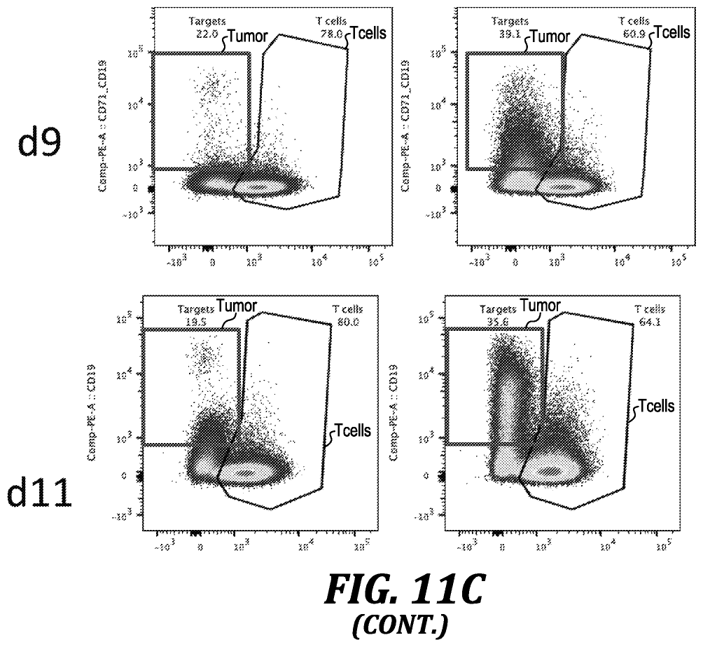

FIGS. 11A and 11B graphically illustrate results of the repeated antigen exposure assays. As illustrated in FIG. 11A, the CD8+ CAR T cells that were cultured with N1 anti-Notch receptor antibody (a Notch agonist) retained significantly higher normalized cells numbers than the control cell cultures after the second and third administrations of target K562-CD19 cells. With respect to administration of Raji cells, as illustrated in FIG. 11B, the CD8+ CAR T cells cultured with N1 antibody exhibited higher normalized cells numbers than the control cell cultures after the first compared to the IgG1 control culture. Furthermore, the CD8+ CAR T cells cultured with N1 antibody exhibited significantly higher numbers compared to both control cultures after the second and third administrations of target Raji cells. FIG. 11C graphically illustrate that CD8⁺ CAR T cells that were cultured with N1 anti-Notch receptor antibody eliminate CD19+ tumor cells more efficiently in the repetitive stimulation assay than those cultured with control IgG1.

Conclusion: These results demonstrate that induced Notch signaling in CAR T cells results in enhanced persistence, and more specifically a lack of exhaustion in environments of continued and chronic target antigen exposure. The CAR T cells exposed to Notch agonist maintained higher proliferative activity in the constant presence of cells expressing the target antigen compared to similar CAR T cells that were not exposed to Notch agonist. Considering that all assessed CAR T cells expressed the same CD19 specific CAR, the reduced susceptibility to exhaustion is attributable to the induction of Notch signaling in the T cells. Accordingly, this indicates that induced Notch signaling is applicable to T cells expressing other CARs to reduce susceptibility to exhaustion and enhance the proliferative capacity and persistence of the cells in therapeutic applications.

### Example 4

Title: Effect of Induced Notch Signaling in CD4+ CAR T Cells vs. CD8+ CAR T Cells.

Introduction: The above examples demonstrate that induced Notch signaling cultured T cells results in a less differentiated phenotype, yet still permits and even promotes proliferation and functionality of the cells. CAR T cells with induced Notch signaling were demonstrated to result in enhanced antitumor effect and persistence in vivo in murine tumor models. Furthermore, in vitro assays demonstrated that CAR T cells with induced Notch signaling exhibited increased persistence and resistance to exhaustion when presented with repeated and prolonged exposure to antigen.

This example describes assays demonstrating that induced Notch signaling has independent effects on CD4+ T cells and CD8+T cells, and mixing populations of CD4+ T cells and CD8+T cells with induced Notch signaling provides a synergistic effect in anti-tumor response.

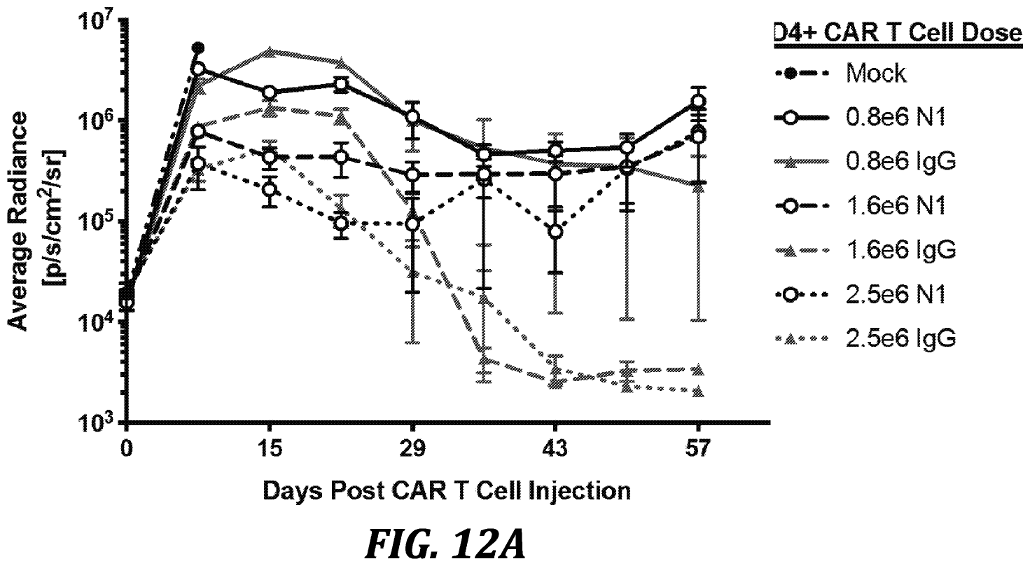

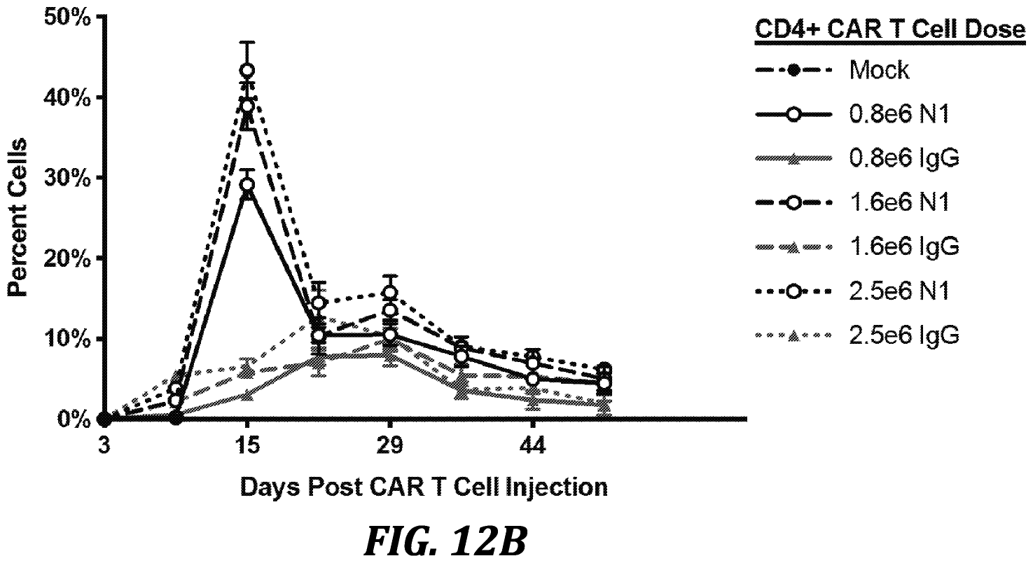

Results and Discussion: First, the effects of induced Notch signaling on CD4+ T cells were examined. CD4+ CAR T cells, as described above, were cultured on plates coated with 2.5 μg of anti-Notch antibody (N1) or IgG1. After 11 days of culture, the N1-exposed CD4+ CAR T cells were infused at different doses into NSG-Raji mice, as described above. Tumor burden was assessed by bioluminescence imaging using firefly luciferase. As graphically illustrated in FIG. 12A, there is no detectable difference in antitumor efficacy by infusing only N1-exposed CD4+ CAR T cells. The levels of CAR T cells were assessed at several time points after infusion. Specifically, blood was collected at various claim points after the infusion of N1-exposed CD4+ CAR T cells. The blood was lysed with ammonium chloride potassium solution and then stained with antibodies for CD45, CD4, and EGFR. Data was collected on a Canto 2-1 flow cytometer. These assays revealed that the mice treated with N1-exposed CD4+ CAR T cells have much higher frequencies of EGFR+ CD4+ T cells than control groups at d15, which contract by d22. Accordingly, CD4+ CAR T cells exhibit strong proliferative capacity as a result of Notch signaling induction. However, this alone does not lead to significantly enhanced antitumor effect.

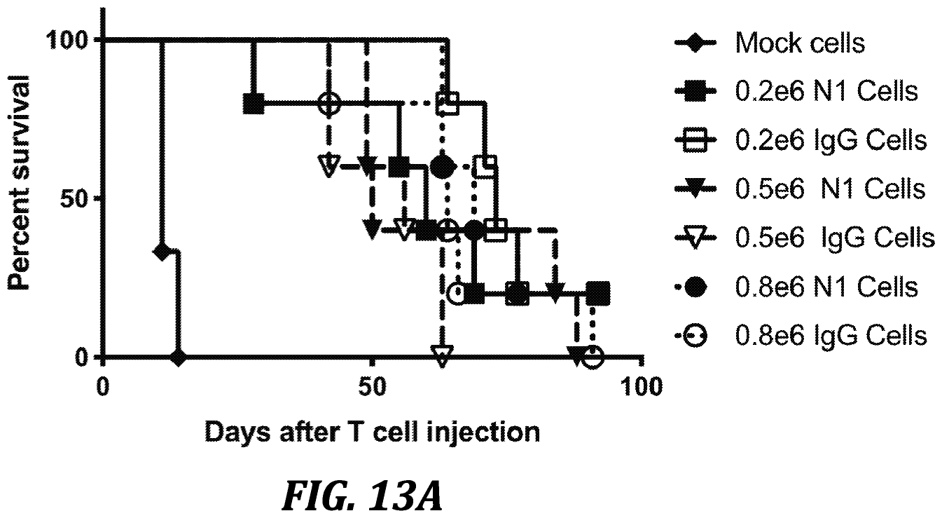

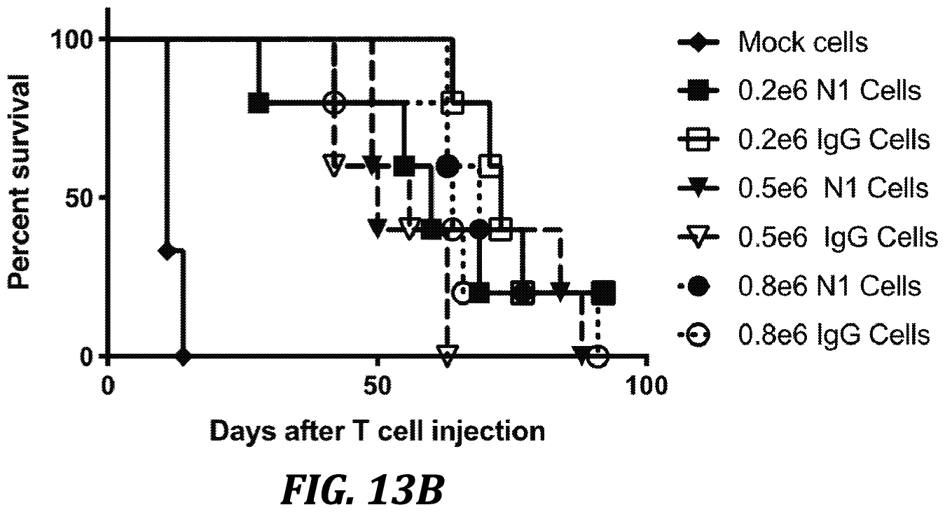

Similar assays were performed for CD8+ CAR T cells. CD8+ CAR T cells, as described above, were cultured on plates coated with 2.5 μg anti-Notch antibody (N1) or IgG1 as a control. After 11 days of culture, the N1-exposed CD8+ CAR T cells were infused at different doses into NSG-Raji mice, as described above. Tumor burden was assessed by bioluminescence imaging using firefly luciferase. As graphically illustrated in FIG. 13A, enhanced antitumor effect was observed after infusion of only N1-exposed CD8+ CAR T cells, although this difference of effect decreased over time. The levels of CD8+ CAR T cells were assessed at several time points after infusion, as described above. Interestingly, there was no clear peak expansion of the CD8+ CAR T cells due to incubation with N1 antibody. The levels of CD8+ CAR T cells at their peak were less than about 2% of lymphocyte singlets, in contrast to the CD4+ CAR T cells, which achieved a peak of about 30 to 45% on day 14 post-infusion. Survival of the NSG-Raji mice infused with the N1-exposed CD8+ CAR T cells was assessed. As illustrated in FIG. 13B, there is little extended survival of NSG-Raji mice infused with the N1-exposed CD8+ CAR T cells versus NSG-Raji mice infused with CD8+ CAR T cells exposed to the IgG control.

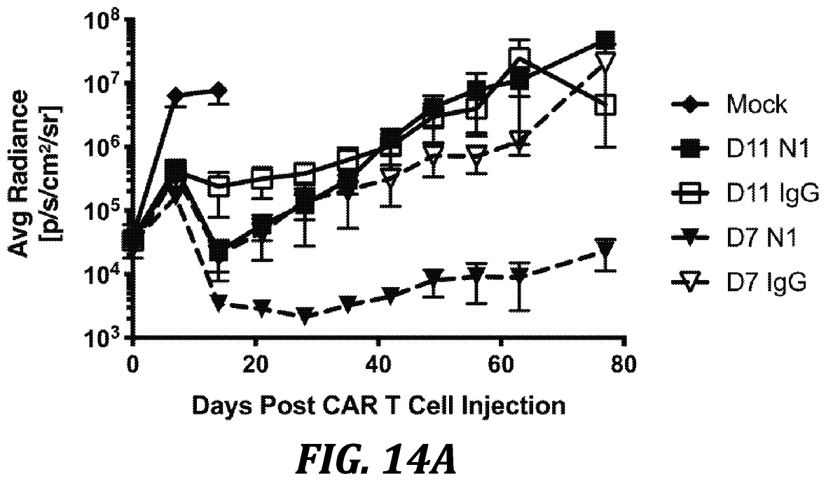

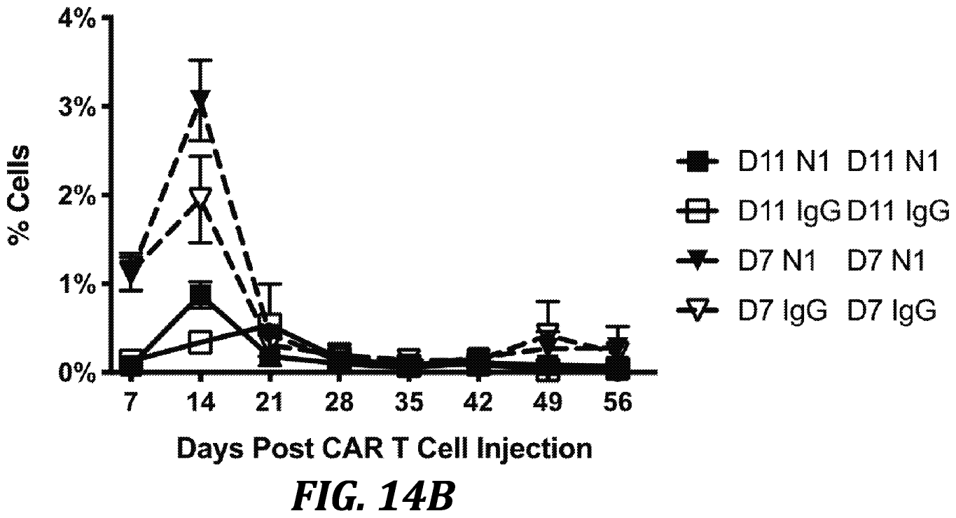

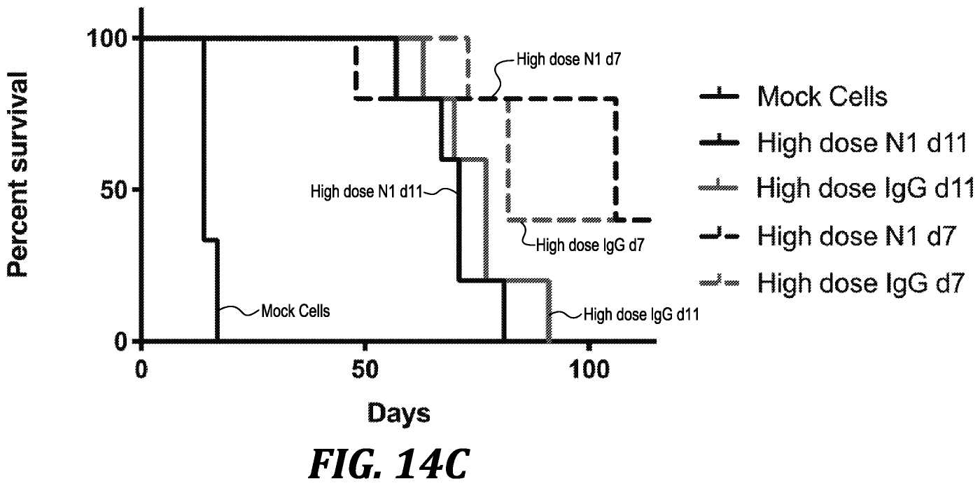

For further assessment, two different cultures of CD8+ CAR T cells were examined. CD8+ CAR T cells were cultured on plates with N1 antibody agonist or IgG1 for 7 days or for seven days followed by four additional days in normal tissue culture flasks (D11 group). The CD8+ CAR T cells were infused into NSG-Raji mice, as described above. The effect of the different infusions on tumor burden was tested by bioluminescence imaging of firefly luciferase overtime. As illustrated in FIG. 14A, there was enhanced reduction of tumor burden resulting from infusion of CD8+ CAR T cells exposed to N1 agonist for seven days. Furthermore, as illustrated in FIG. 14B, CD8+ CAR T cells exposed to N1 agonist for seven days exhibited increased proliferation in vivo, peaking around day 14 post-infusion. However, as illustrated in FIG. 14C, these enhancements of CD8+ CAR T cell functionality did not result in increased survival compared to CD8+ CAR T cells exposed to IgG controls.

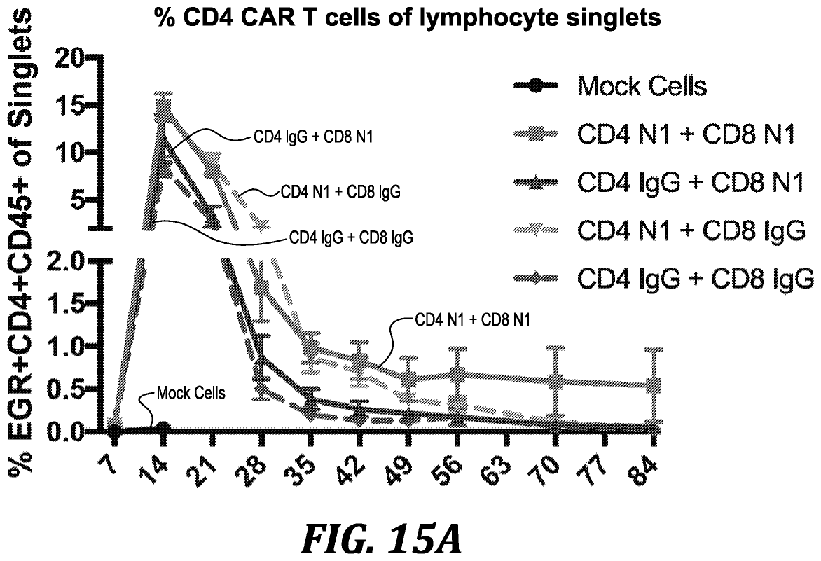

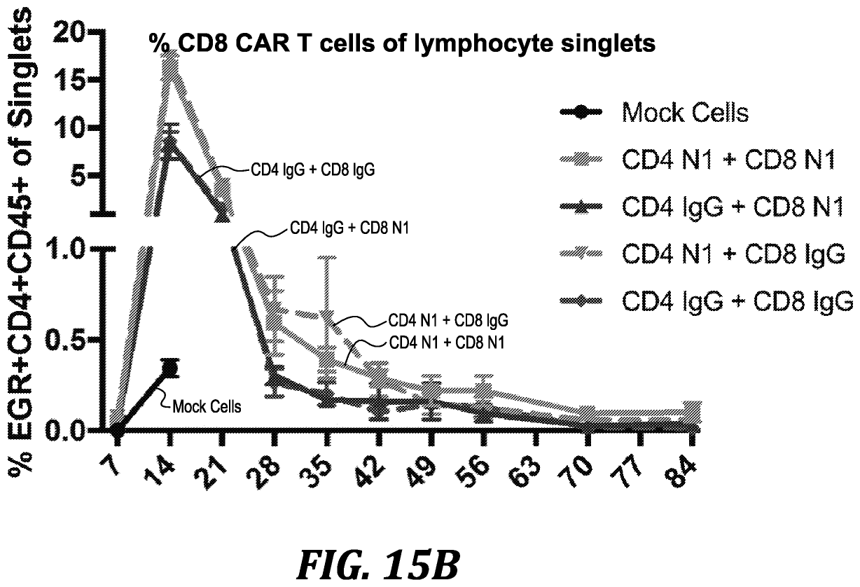

These results indicate that induced Notch signaling has independent or different effects on CD4+ T cells and CD8+ T cells. The assays addressing CD4+ T cells demonstrated that exposure to Notch agonist in culture results in higher proliferative capacity of the CD4+ T cells, with the peak expansion between days 10 and 14 post-infusion exceeding 40% of CAR T cells singlets versus less than 10% of CAR T cells exposed to the IgG control. However, despite the relatively high level of expansion in vivo, this did not lead to measurable improvement antitumor efficacy. In contrast, the assays addressing CD8+ T cells demonstrated that exposure to Notch agonist in culture results a relatively minor T cell expansion peak after infusion. Interestingly, the Notch-induced CD8+ T cells exhibited better tumor control after infusion although the tumor eventually grows to within equivalents of control CD8+ T cell infusions. In view of these independent effects, coordination of CD4+ T cells and CD8+ T cells with Notch signaling induction was examined. Four cultures of CAR T cells were separately generated: CD4+ CAR T cells exposed to Notch agonist, N1 antibody; CD4+ CAR T cells exposed to control IgG1 antibody; CD8+ CAR T cells exposed to Notch agonist, N1 antibody; and CD8+ CAR T cells exposed to control IgG1 antibody. Combinations of the CD4+ and CD8+ cultures were mixed together on day 11 at a 1:1 ratio and were infused into NSG-Raji mice. See, e.g., Hudecek, M., et al., The Nonsignaling Extracellular Spacer Domain of Chimeric Antigen Receptors Is Decisive for In Vivo Antitumor Activity, Cancer Immunology Research, 125-135 (2015), incorporated herein by reference in its entirety, for disclosure of application of CD19-CAR T cells in NSG-Raji mice. Proliferation of the CD4+ CAR T cells and CD8+ CAR T cells were determined overtime for each combination. As illustrated in FIGS. 15A and 15B, infusions where CD4+ CAR T cells previously exposed to Notch agonist resulted in the greatest proliferation of both CD4+ CAR T cells and CD8+ CAR T cells, regardless of whether the CD8+ CAR T cells were previously exposed to Notch agonist. This demonstrates that induced Notch signaling in CD4+ T cells during culture increases proliferation and prolonged persistence of both CD4+ CAR T cells and CD8+ CAR T cells, and the remarkable antitumor efficacy observed in the examples described above.

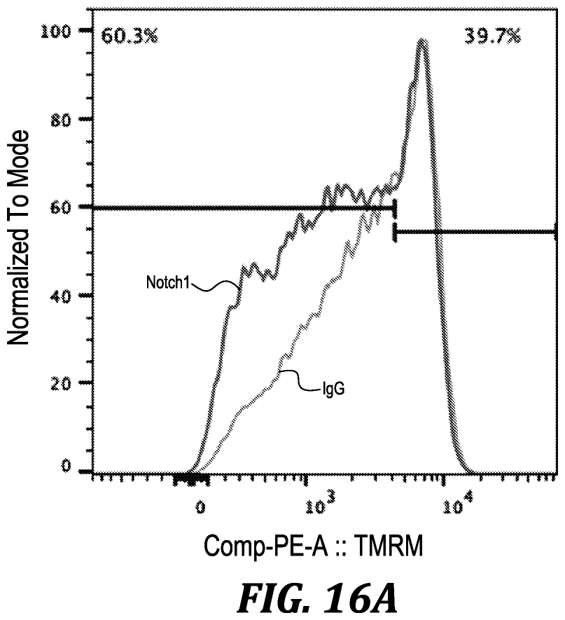

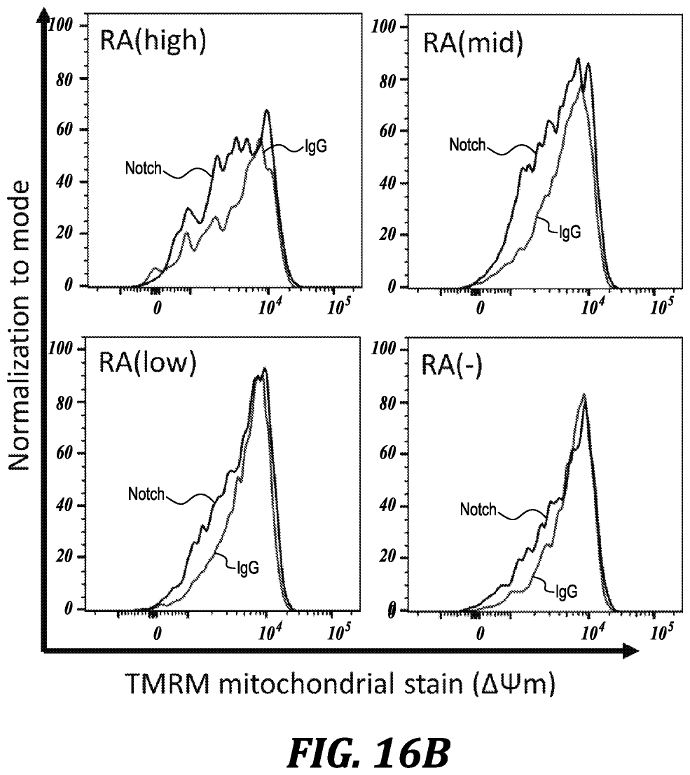

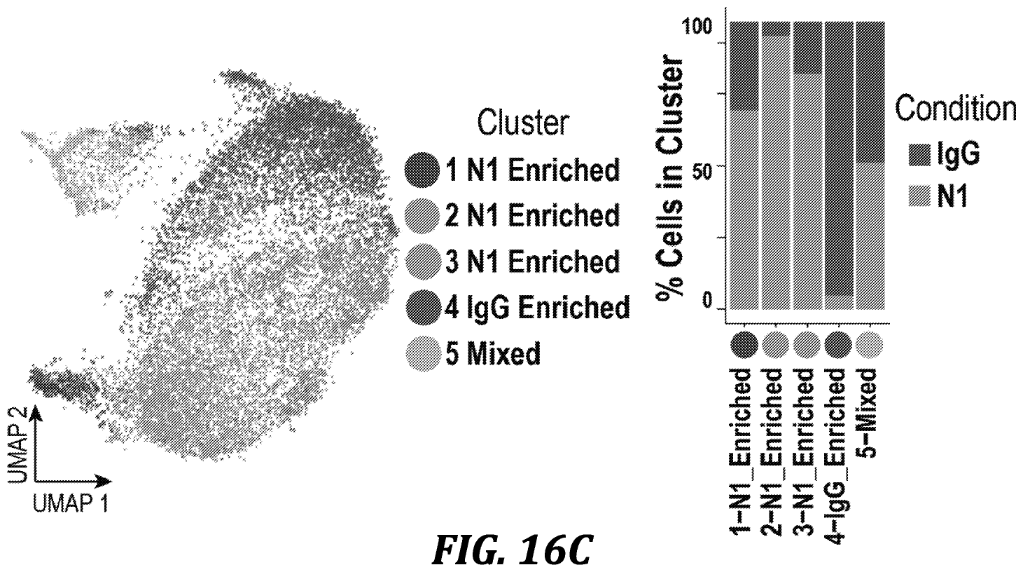

To further characterize the phenotypes of CD4+ CAR T cells incubated with N1 agonist antibody, a further assay was performed using a tetramethylrhodamine, methyl ester (TMRM) stain to label the mitochondria in the CD4+ CAR T cells with induced Notch signaling. Naive CD4+ T cells were cultured on plates with 2.5 μg immobilized N1 anti-Notch antibody for 11 days. As illustrated in FIG. 16A, CD4+ CAR T cells incubated with N1 antibody had lower mitochondrial membrane potential, indicating increased metabolic fitness. Further flow cytometric analysis indicated that such reduced mitochondrial membrane potential induced by Notch signaling is mainly in CD45RA high/CD45RO low CD4 cells. See FIG. 16B. Ultimately, induction of Notch signaling in culture results in distinct molecular profiles on the CD8+ T cell surface. See e.g. FIG. 16C.

Conclusion: These results demonstrate that induced Notch signaling in T cells affects CD4+ T cells and CD8+ T cells differently. In isolation, CD4+ T cells are induced by Notch signaling to have greater proliferation, but also the exhibit less antitumor effect. In contrast, CD8+ T cells are induced by Notch signaling to have greater initial antitumor effect, but do not exhibit significant proliferation. However, when combined CD4+ T cells and CD8+ T cells that have been previously exposed to Notch agonist in culture exhibit high proliferation, prolonged persistence, and enhanced in particular effect. The results indicate that induced Notch signaling, specifically in CD4+ T cells, can lead to this effect.

### Example 5

Title: Comparison of the Differentiation State of Starting T Cell Populations on the Impact of Notch Signaling in Cultured T Cells.

Introduction: It is demonstrated above that induced Notch signaling in T cells maintains an early, i.e. relatively undifferentiated, state while promoting proliferative and antitumor capabilities. This results in significantly increased longevity and resistance to exhaustion in vivo. It is also demonstrated that Notch signaling affects CD4+ T cells and CD8+ T cells differently, and that some of the remarkable qualities observed from Notch stimulated T cells results from synergistic cooperation of Notch exposed CD4+ T cells and CD8+ T cells.

The results obtained in the above examples were based on assays using isolated naïve (i.e., T_{N}) T cells. In this Example, the effects of induced Notch signaling were investigated for different initial T cell subsets.

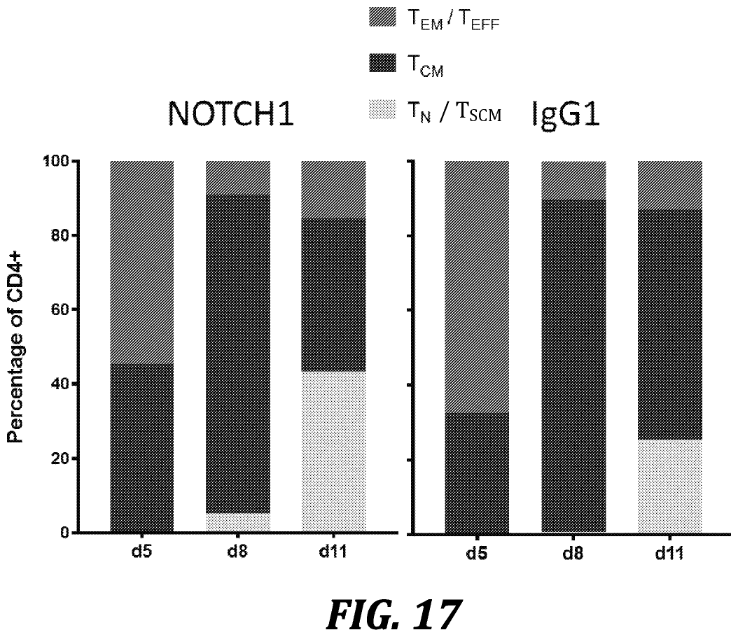

Results and Discussion: In the first assay, peripheral blood mononuclear cells (PBMCs) were sorted to provide a population of CD4+ T cells depleted of naïve (i.e., T_{N}) T cells. CD45RO+ CD4T cells were isolated by a method wherein antibodies for other markers (i.e., CD8 and CD45RA) were contacted to the cells, and bound cells were depleted by magnetic activated cell sorting. The retained cells were CD45RO+, CD62L+ and CD62L−, and CD4+. The resulting cell population was incubated on plates coated with anti-Notch1 antibody, i.e. N1 agonist antibody, IgG1 control, or tissue culture control without ligand. At days 5, 8, and 11 after start of the culture, the cell population starting from CD4+ naïve depleted cells were assessed for expression of CD28, CD27 and CD62L to ascertain developmental state of the cells. By day 5, the cells exposed to N1 agonist had higher expression of CD28 and CD62L compared to cells exposed to IgG control. By day 8, the cells exposed to N1 agonist had lower expression of CD35 and higher expression of CD27 compared to cells exposed to IgG control. FIG. 17 graphically illustrates the percentage of the CD4+ T cells from days 5, 8, and 11 of culture that are categorized as T_{EM}/T_{EFF}, T_{CM}, or T_{N}/T_{SCM} based on the expression profile of the developmental markers. As illustrated, induced Notch signaling results in maintenance of less differentiated states in CD4+ T cells that have already started to differentiate (i.e., CD4+ T cells that have been initially depleted of undifferentiated CD4+ T cells (T_{N})) as compared to the IgG control.

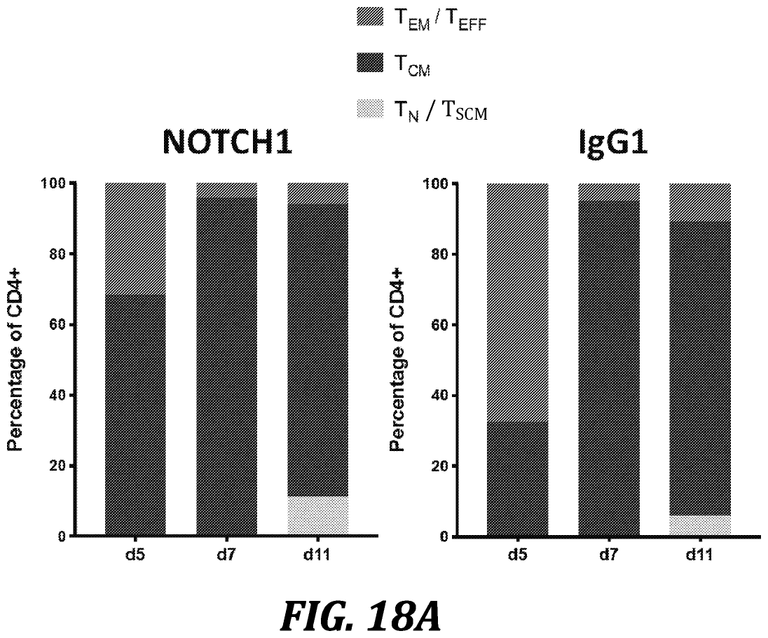

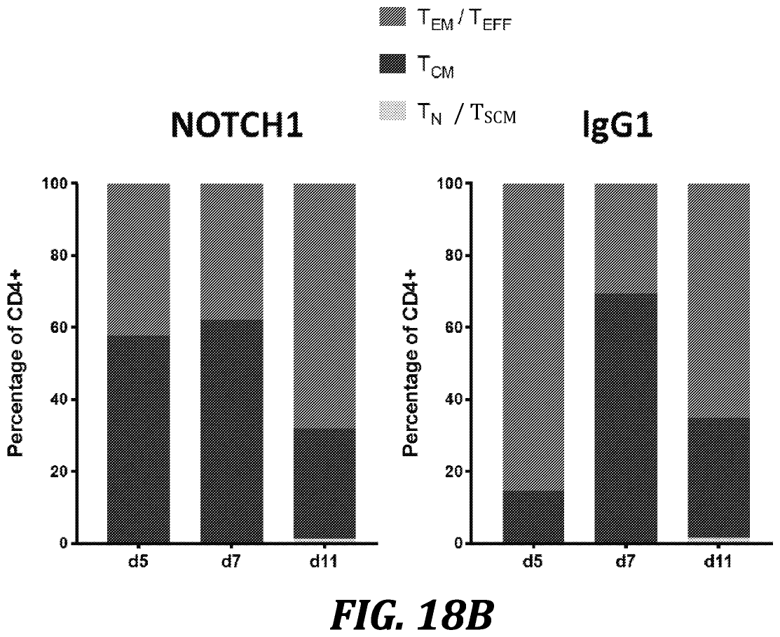

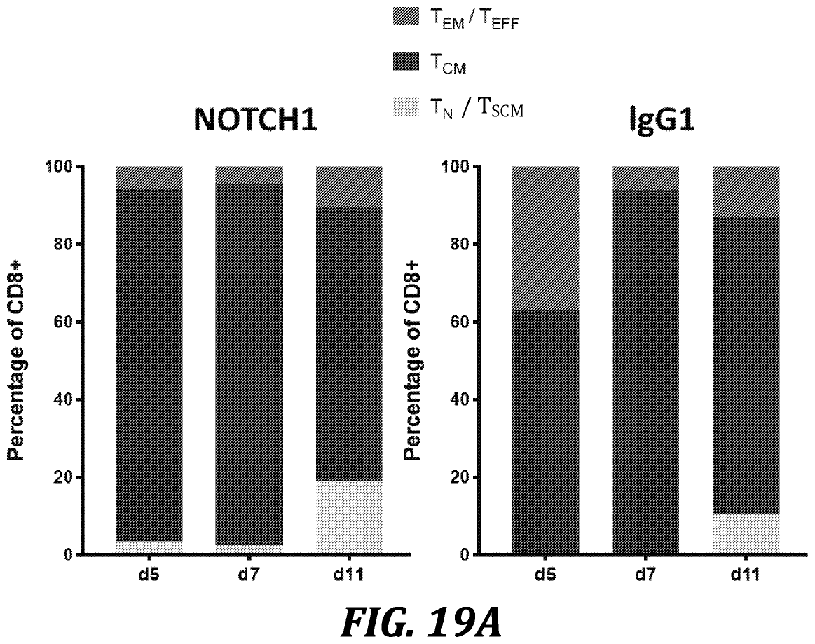

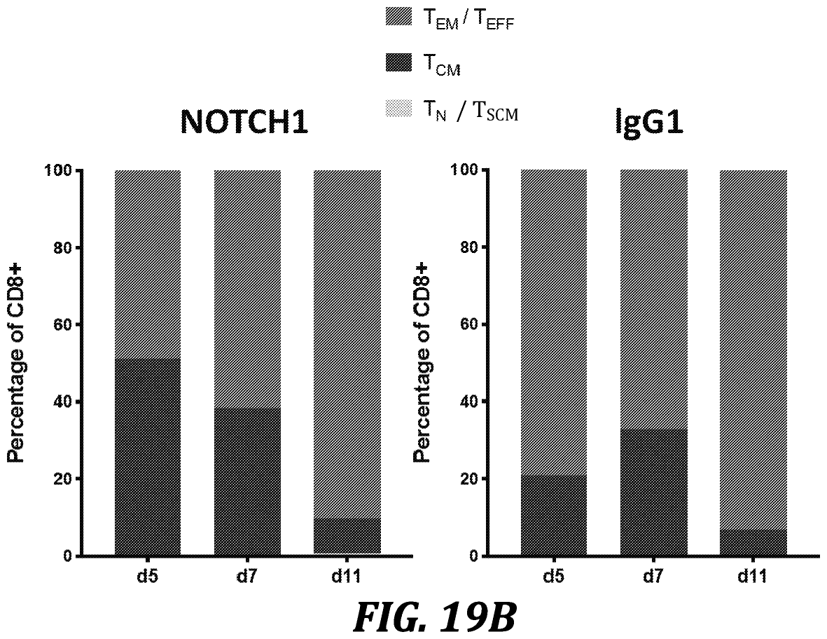

Next, initial PBMC populations were sorted for CD4+ T_{CM} cells, CD8+ T_{CM} cells, CD4+ T_{EM} cells, and CD8+ T_{EM} cells. Briefly, CD4+ and CD8+ T cells were isolated from PBMC using magnetic activated cell sorting. Subsequently, CD4+ and CD8+ T cells were labeled with antibodies for CD45RO and CD62L and sorted by flow cytometry based on expression of these markers into T_{N}/T_{SCM} (CD45RO−CD62L+), T_{CM} (CD45RO+/CD62L+) and T_{EM} (CD45RO+CD62L−). The different sorted cell populations were separately incubated on plates coated with anti-Notch1 antibody, i.e. N1 agonist antibody, IgG1 control, or tissue culture control without ligand. At days 5, 8, and 11 after start of the culture, the cell populations were assessed for expression of CD27, CD28, CD62L, PD-1, and CD25 markers to ascertain developmental and activation state of the cells after incubation. As illustrated in FIG. 18A, CD4+ T_{CM} cells incubated with Notch receptor agonist maintained a significantly higher proportion of T_{CM} cells at day 5 compared to CD4+ T_{CM} cells incubated with IgG1 control, which had a higher proportion of more developed T_{EM}/T_{EFF} cells. By day 11, the Notch-induced culture had higher proportion of T_{N} cells. As illustrated in FIG. 18B, CD4+ T_{EM} cells incubated with Notch receptor agonist developed a significantly higher proportion of T_{CM} cells at day 5 compared to CD4+ T_{CM} cells incubated with IgG1 control. As illustrated in FIG. 19A, CD8+ T_{CM} cells incubated with Notch receptor agonist maintained a significantly higher proportion of T_{CM} cells at day 5, and even developed detectable T_{N} cells, compared to CD8+ T_{CM} cells incubated with IgG1 control, which had a higher proportion of more developed T_{EM}/T_{EFF} cells. By day 11, the Notch-induced culture had higher proportion of T_{N} cells compared to the IgG1 control group. As illustrated in FIG. 19B, CD8+ T_{EM} cells incubated with Notch receptor agonist developed a significantly higher proportion of T_{CM} cells at day 5 compared to CD8+ T_{CM} cells incubated with IgG1 control.

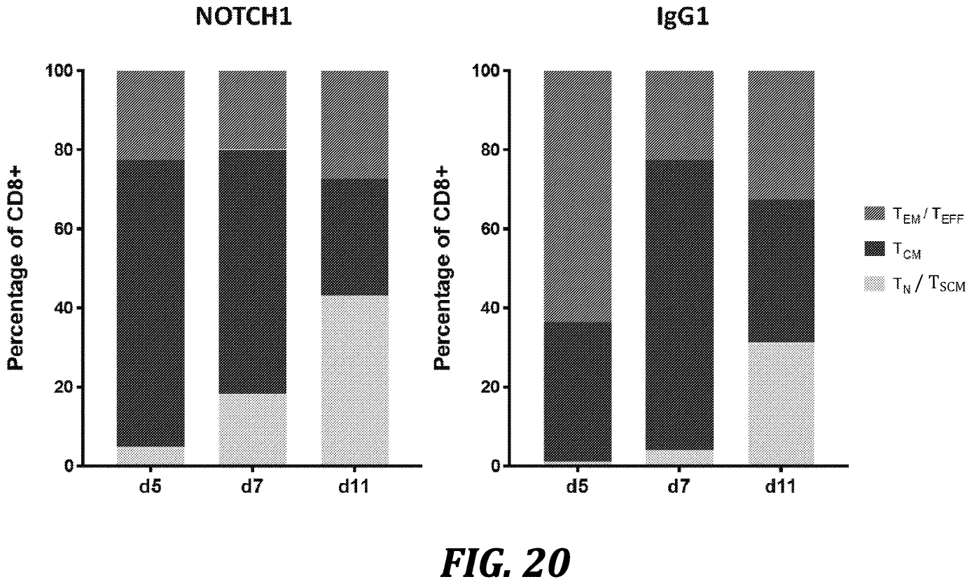

Considering that the effect of induced Notch signaling on the different sorted T cell subsets with similar, the effect on all cultures was tested. The bulk CD8+ T cells were sorted and cultured in anti-Notch1 agonist antibody, N1, or IgG1 antibody control. Cells were tested at days 5, 7, and 11 after initiation of the culture for expression of developmental cell markers CD28, CD27, CD62L, PD-1, LAG3, and CD25. As illustrated in FIG. 20, after five days of culture, the bulk cells cultured with N1 antibody had a significantly higher proportion T_{CM} cells, and detectable T_{N} cells, compared to the IgG control group which had a higher proportion of T_{EM}/T_{EFF} cells. Over time, the bulk cells cultured with N1 antibody developed a higher proportion of T_{N} cells compared to the IgG1 control group.

Conclusion: These data demonstrate that culturing of T cells with Notch1 agonist had the same effect regardless of the developmental state of the T cells. More specifically the same effects were observed in naïve T cells, naïve depleted T cells, sorted T cell subsets (including for CD4+ and CD8+ T cells), and bulk T cells. Regardless of the grouping, exposure to Notch receptor agonist resulted in maintenance or development of a less differentiated state in the cell populations, as indicated by higher expression of CD28 and CD27, and lower expression of PD-1, LAG-3 and CD25. Accordingly the effects of Notch signaling as demonstrated above is not restricted to any particular T cell developmental state.

### Example 6

Title: Exemplary Assays Demonstrating Persistence and Lack of Exhaustion in the Context of Chronic Antigen Exposure In Vivo for CAR T Cells with Induced Notch Signaling.

Introduction: As indicated above, a major obstacle to therapeutic applications of CAR T cells in anti-viral and anti-cancer settings is their exhaustion, i.e., limited temporal effector efficacy, during prolonged or chronic exposure to target antigen, thus limiting efficacy of the adoptive CAR T therapeutic approach.

It is demonstrated above that CAR T cells that have been generated in the presence of Notch agonist results in a less differentiated phenotype for a prolonged period but without inhibition of proliferation and functionality. These Notch-induced CAR T cells were shown to have enhanced efficacy and persistence in vivo in NSG mice engrafted with Raji lymphoma, resulting in significantly reduced tumor burden and enhanced survival over prolonged periods compared to adoptive transfer of control T cells, and control CAR T cells that were not exposed to Notch agonist. Finally, in vitro studies showed that Notch-induced CAR T cells had less susceptibility to exhaustion in an environment with repeated and persistent exposure to the target antigen. The cultured Notch-induced CAR T cells maintained higher proliferative activity in the constant presence of cells expressing the target antigen compared to similar CAR T cells that were not exposed to Notch agonist.

This Example describes exemplary assays to confirm the reduced susceptibility to exhaustion of Notch-induced CAR T cells in vivo.

Experimental Design:

Experimental and control T cells can be generated as described in more detail above, i.e., including culture steps on plates with Notch ligand, IgG1 control ligand, or tissue culture alone). The cultures can also include anti-CD3/CD28 DYNABEADS® (ThermoFisher) at a 3:1 ratio for activation. Culture can then incorporate transduction to engineer for CAR (e.g., anti-CD19 or ROR1) or TCR expression.

In one exemplary assay, the reduced susceptibility of Notch-induced CAR T cells can be addressed in a Raji-ffluc model, as generally described in more detail in Hudecek, M., et al., Cancer Immunol Res., 2015, 3(2):125-135, incorporated herein by reference in its entirety. Briefly, Raji cells can be transduced with lentivirus encoding an ffluc/eGFP fusion gene to allow labeling of the cells, and then sorted for expression of eGFP to derive the Raji-ffluc cells. As indicated above, ˜5×10⁵ Raji-ffluc cells can be engrafted in NOD/SCID/γ chain−/− (NSG) mice to create a murine lymphoma model. After a designated period of time, for example 7 days, the mice are administered Notch-induced CD19 CAR T cells, IgG1-incubated CAR T cells (irrelevant antigen control), or tissue culture control CAR T cells. After administration of the CAR T cells, the NSG mice receive one or more additional administrations of Raji-ffluc cells to ensure continued exposure of the administered CAR T cells to the cognate antigen.

In another exemplary assay, the reduced susceptibility of Notch-induced CAR T cells can be addressed in a ROR1+ murine model, as generally described in more detail in Hudecek, M., et al., Clin Cancer Res., 2013, 19(12):3153-3164, incorporated herein by reference in its entirety. Briefly, the receptor tyrosine kinase-like orphan receptor 1 (ROR1) is a candidate for immunotherapy that has been addressed with CAR-modified T-cells. ROR1 is a 120-kDa glycoprotein containing extracellular immunoglobulin (Ig)-like, Frizzled, and Kringle domains. ROR1 is expressed during embryogenesis but is absent from normal adult tissues, apart from a subset of immature B-cell precursors, and low-level expression on adipocytes. ROR1 was first shown to be expressed in B-cell chronic lymphocytic leukemia (B-CLL) by transcriptional profiling, and was subsequently identified on the surface of many cancers including mantle cell lymphoma (MCL), acute lymphoblastic leukemia (ALL) with a t(1;19) chromosome translocation, and a subset of lung, breast, colon, pancreas, renal, and ovarian cancers (14-21). In both lung adenocarcinoma and t(1;19) ALL, ROR1 cooperates in oncogenic signaling, and knockdown of ROR1 with siRNA exposed a critical role for this molecule in maintaining tumor cell survival.

For the exemplary assay, K562/ROR1 and Raji/ROR1 cells are generated by lentiviral transduction of the cells with the full-length ROR1 gene. Anti-CD19 and anti-ROR1 CAR expressing T cells are generated as described in more detail above, including exposure to Notch-1 ligand or appropriate control. NSG mice receive an initial administration of the K562/ROR1 and Raji/ROR1 cells (e.g., ˜0.5×10⁶ cells). After a period of time, e.g., 7 days, the mice receive administrations of the Notch-induced CAR T cells, or the control CAR T cells alternatively incubated with the appropriate controls. After administration of the CAR T cells, the NSG mice can receive one or more additional administrations of K562/ROR1 or Raji/ROR1 cells to ensure continued exposure of the administered CAR T cells to the cognate antigen.

In either assay design, the T cells can be sampled from the mice over time from the mice and assayed/monitored for proliferation state, cytotoxicity, and cytokine production, as described in more detail above. Furthermore, the mice can be monitored for tumor burden using, e.g., bioluminescence analysis, and for survival in the context of repeated administration of Raji-ffluc cells, as described in more detail above.

Expected Results

In view of the above results demonstrating prolonged survival, persistence, and activation within the subject, and reduced susceptibility to exhaustion in environments of continued or repeated exposure to antigen, the present assays are expected to demonstrate that the administered Notch-induced CAR T cells maintain a less-differentiated state over prolonged periods of time in vivo compared to the CAR T cells that did not receive Notch signaling during culture. This prolonged persistence is expected to manifest in a reduced rate of exhaustion during prolonged and/or repeated antigen exposure in vivo leading to enhanced reduction and/or clearance of tumor burden and increased survival of the subject.

### Example 7

Title: Exemplary Methods and Materials to Harvest, Culture, Modify, and Characterize T Cells.

Cells

Naïve T cells can be isolated using the EASYSEPTM Human Naive CD4+ and CD8+ T Cell Isolation kits (Stem Cell Technologies, catalog #19555/19258, respectively) or EASYSEP™ Human Memory CD4+T Cell Enrichment kit (Stem Cell Technologies, catalog #19157).

Coating Plates with Notch1 Antibody

PBS is used to dilute antibodies for coating. Notch1 wells are coated with 2.5 μg/mL LEAF-purified Notch1 antibody (Biolegend catalog #352104), DLL1, DLL4, or N2 antibody (Biolegend catalog #348301). IgG wells are coated with 2.5 μg/mL human IgG. Retronectin (Takara) is used at 5 μg/ml for coated wells. The coating volume used is 0.5 mL per well in a 24 well format, 1 mL per well in a 12 well format, and 2 mL per well in a 6 well format.

Preparation of Virus

Concentrated fmc63 (anti-CD19-41BB CAR) lentivirus is generated using the packaging and envelope vectors PCHGP-2, pCMV-Rev2, and pCMV-G with the calcium-phosphate transfection protocol. After three days of virus production, virus was concentrated using PEG and ultracentrifugation, and then resuspended in DMEM. Viral titer was measured before use.

Transduction

On the day before transduction, cells are plated at a density of 2e5 cells/well in Notch1/retronectin-, human IgG/retronectin-, or TC-treated plates. Cells are cultured with anti-CD3/CD28 DYNABEADS® (ThermoFisher) at a 3:1 ratio and 50 u/mL IL2 for 24 hours before transduction. The DYNABEADS® are then removed to avoid overstimulation. Cells are transduced by centrifugation at 800×g for 90 minutes at 32 C using concentrated fmc63 virus, polybrene (0.43 μg/mL), and 50 IU/ml IL2. A half-media change is performed 6 hours after transduction.

Culturing

At 72 hours after T cell stimulation (48 hours after transduction), cells are transferred to coated or TC-treated 12 well plates and given a half-media change plus an additional 500 uL media. Five days after T cell stimulation, magnetic DYNABEADS® are removed and cells are transferred to coated or TC-treated 6 well plates. At day 7 after T cell stimulation, cells are transferred to normal tissue culture T25 or T75 flasks. (1 well to one T25 flask or 3 wells to one T75 flask.)

In Vitro Assays

Proliferation (as in Receptor Affinity and Extracellular Domain Modifications Affect Tumor Recognition by ROR1-Specific Chimeric Antigen Receptor T Cells by Michael Hudecek, Maria-Teresa Lupo-Stanghellini, Paula L. Kosasih, Daniel Sommermeyer, Michael C. Jensen, Christoph Rader and Stanley R. Riddell DOI: 10.1158/1078-0432.CCR-13-0330 Published June 2013).

T cells were labeled with 0.2 μmol/L carboxyfluorescein succinimidyl ester (CFSE; Invitrogen), washed, and plated in triplicate with stimulator cells in medium without exogenous cytokines. After 72-hour incubation, cells were labeled with anti-CD4 or CD8 mAb, anti-EGFR mAb and propidium iodide, and analyzed by flow cytometry to assess cell division of live CD4/CD8+ T cells.

For phenotype and activation analysis in vitro and for mouse bleed analysis, cells were stained with monoclonal antibodies for 20 min, followed by a wash in flow buffer and data acquisition on a Canto II flow cytometer. Data was analyzed in Flowjo (Treestar).

While illustrative embodiments have been illustrated and described, it will be appreciated that various changes can be made therein without departing from the spirit and scope of the invention.

1. A method of culturing a naïve T (T_{N}) cell in vitro, comprising exposing a naïve T (T_{N}) cell to a medium comprising Notch receptor agonist for a time sufficient to induce Notch receptor signaling in the cell.

2. The method of claim 1, wherein the method comprises exposing a population of cells comprising a plurality of naïve T (T_{N}) cells to a medium comprising Notch receptor agonist for a time sufficient to induce Notch receptor signaling in the plurality of T_{N} cells.

3-4. (canceled)

5. The method of claim 1, wherein the T_{N} cell is further characterized as CD62L+, CD45RA+, CD45RO−, CD95−, and/or CCR7+.

6. The method of claim 1, wherein the exposing lasts for a period (“the exposure time”) of at least about 12 hours.

7-8. (canceled)

9. The method of claim 2, wherein the percentage of the T_{N} cells in the population changes, after the exposing, by less than about 50%.

10-11. (canceled)

12. The method of claim 1, wherein the Notch receptor agonist comprises a domain of a mammalian Notch receptor ligand that binds to a mammalian Notch1, Notch2, Notch3, or Notch4 receptor.

13. The method of claim 1, wherein the Notch receptor agonist is or comprises a Delta protein, a Jagged protein, an anti-Notch antibody, or a fragment or derivative thereof, that binds to a mammalian Notch receptor, or any combination thereof.

14. The method of claim 1, wherein the Notch receptor agonist, upon binding to a Notch receptor, induces a conformational change of the Notch Receptor resulting in exposure of an S2 cleavage site in the negative regulatory region (NRR) of the Notch receptor.

15-16. (canceled)

17. The method of claim 13, wherein the Notch receptor agonist is or comprises Delta Like Ligand 1 (DLL1), Delta Like Ligand 3 (DLL3), Delta Like Ligand 4 (DLL4), Jagged1, Jagged2, Dlk1, Dlk2, DNER, EGFL 7, F3/contactin, a fragment thereof, a derivative thereof, or any combination thereof.

18-20. (canceled)

21. The method of claim 13, wherein the anti-Notch antibody or an antigen binding fragment thereof binds to an epitope in the Notch extracellular domain (NECD) that is not in the negative regulatory region (NRR) of the Notch receptor.

22. (canceled)

23. The method of claim 1, wherein the Notch receptor agonist is present at a concentration of about 0.01 μg/ml to about 100 μg/ml.

24. The method of claim 1, wherein the Notch receptor agonist is immobilized on a surface or scaffold.

25. (canceled)

26. The method of claim 1, wherein the medium further comprises one or more cytokines, or a biologically active fragment thereof, in an effective concentration that modulates T_{N} cell differentiation, wherein the one or more cytokines is selected from IL-1, IL-lb, IL-2, IL-4, IL-6, IL-7, IL-10, IL-12, IL-15, IL-17, IL-21, IL-23, IL-27, IFN-γ, TNF-α, and TGFβ.

27-28. (canceled)

29. The method of claim 1, further comprising isolating the T_{N} cell, or one or more progeny cells thereof, from the medium after the exposure.

30. The method of claim 2, wherein the method further comprises isolating the population of TN cells, or one or more progeny cells thereof, from the medium after exposure, and at least about 40% of the population of the T_{N} cells or one or more progeny cells thereof after the exposure are T cells having the characteristics of CD62L+ and CD45RO−.

31. The method of claim 1, further comprising transducing the T_{N} cell, or one or more progeny cells thereof, with a heterologous nucleic acid molecule comprising a sequence that encodes an immune receptor.

32. The method of claim 31, wherein the immune receptor is or comprises an antigen receptor that comprises an extracellular domain that specifically binds to an antigen of interest, wherein the extracellular domain is operatively linked to an intracellular domain that activates the T cell upon binding of the extracellular domain to the antigen of interest.

33. The method of claim 31, wherein the immune receptor is a T cell receptor (TCR) that specifically binds to a peptide of interest bound to a major histocompatibility complex (MHC) molecule or a chimeric antigen receptor.

34. The method of claim 29, further comprising administering the T_{N} cell, or one or more progeny cells thereof, to a subject in need thereof.

35-39. (canceled)

40. The method of claim 34, wherein the subject has a condition selected from cancer, infectious disease, and autoimmune disease.

41. A T cell produced by the method of claim 1.

42. A therapeutic composition comprising a plurality of cells recited in claim 41 and an effective carrier.

43. (canceled)

44. A method of reducing or preventing exhaustion of a T_{N} cell expressing a chimeric antigen receptor or of a population of T_{N} cells expressing a chimeric antigen receptor, the method comprising exposing the T_{N} cell or the population of T_{N} cells to a medium comprising a Notch receptor agonist for a time sufficient to induce Notch receptor signaling in the cell.

45. A method of generating a T_{N} cell or a population of T_{N} cells expressing a chimeric antigen receptor, the method comprising modifying the T_{N} cell or the population of T_{N} cells to express a chimeric antigen receptor and exposing the T_{N} cell or a population of T_{N} cells to a medium comprising a Notch receptor agonist, wherein the Notch receptor agonist reduces or prevents exhaustion of the T_{N} cell.

46. (canceled)


SEQUENCE LISTING (17 sequences)

SEQ ID NO: 1 - protein, 685 aa, Rattus rattus
MTPGSRSACRWALLLLAVLWPQRAAGSGIFQLRLQEFANERGMLANGRPCEPGCRTFFRICLKHYQATFSEGPCTFGNVSTPVLGTNSFVIRDKNSGSGRNPLQLPFNFTWPGTFSLNIQAWHTPGDDLRPETSPGNSLISQIIIQGSLAVGKNWKSDEQNNTLTRLRYSYRVVCSDNYYGDSCSRLCKKRDDHFGHYECQPDGSLSCLPGWTGKYCDQPICLSGCHEQNGYCSKPDECNCRPGWQGPLCNECIPHNGCRHGTCTIPWQCACDEGWGGLFCDQDLNYCTHHSPCKNGSTCSNSGPRGYTCTCLPGYTGEHCELELSKCASNPCRNGGSCKDHENSYHCLCPPGYYGQHCEHSTLTCADSPCFNGGSCRERNQGASYACECPPNFTGSNCEKKVDRCTSNPCANGGQCLNRGPSRTCRCRPGFTGTHCELHISDCARSPCAHGGTCHDLENGPVCTCPAGFSGRRCEVRITNDACASGPCFNGATCYTGLSPNNFVCNCPYGFVGSRCEFPVGLPPSFPWVAVSLGVGLVVLLVLLVMVAVAVRQLRLRRPDDDSREAMNNLSDFQKDNLIPAAQLKNTNQKKELEVDCGLDKSNCGKLQNHTLDYNLAPGFLGRGSTPGKYPHSDKSLGEKVPLRLHSEKPACRISAICSPRDSMYQSVCLISEERNECVIATEV

SEQ ID NO: 2 - protein, 257 aa, Rattus rattus
SGIFQLRLQEFANERGMLANGRPCEPGCRTFFRICLKHYQATFSEGPCTFGNVSTPVLGTNSFVIRDKNSGSGRNPLQLPFNFTWPGTFSLNIQAWHTPGDDLRPETSPGNSLISQIIIQGSLAVGKNWKSDEQNNTLTRLRYSYRVVCSDNYYGDSCSRLCKKRDDHFGHYECQPDGSLSCLPGWTGKYCDQPICLSGCHEQNGYCSKPDECNCRPGWQGPLCNECIPHNGCRHGTCTIPWQCACDEGWGGLFCDQ

SEQ ID NO: 3 - protein, 1219 aa, Rattus rattus
MRSPRTRGRPGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPECNKAICRQGCSPKHGSCKLPGDCRCQYGWQGLYCDKCIPHPGCVHGTCNEPWQCLCETNWGGQLCDKDLNYCGTHQPCLNRGTCSNTGPDKYQCSCPEGYSGPNCEIAEHACLSDPCHNRGSCKETSSGFECECSPGWTGPTCSTNIDDCSPNNCSHGGTCQDLVNGFKCVCPPQWTGKTCQLDANECEAKPCVNARSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSESGGKFTCDCNKGFTGTYCHENINDCEGNPCTNGGTCIDGVNSYKCICSDGWEGAHCENNINDCSQNPCHYGGTCRDLVNDFYCDCKNGWKGKTCHSRDSQCDEATCNNGGTCYDEVDTFKCMCPGGWEGTTCNIARNSSCLPNPCHNGGTCVVNGDSFTCVCKEGWEGPICTQNTNDCSPHPCYNSGTCVDGDNWYRCECAPGFAGPDCRININECQSSPCAFGATCVDEINGYQCICPPGHSGAKCHEVSGRSCITMGRVILDGAKWDDDCNTCQCLNGRVACSKVWCGPRPCQLHKGHGECPNGQSCIPVLDDQCFVRPCTGAGECRSSSLQPVKTKCTSDSYYQDNCANITFTFNKEMMSPGLTTEHICSELRNLNILKNVSAEYSIYIACEPSLSANNEIHVAISAEDIRDDGNPVKEITDKIIDLVSKRDGNSSLIAAVAEVRVQRRPLKNRTDFLVPLLSSVLTVAWVCCLVTAFYWCVRKRRRKPSSHTHSAPEDNTTNNVREQLNQIKNPIEKHGANTVPIKDYENKNSKMSKIRTHNSEVEEDDMDKHQQKVRFAKQPVYTLVDREEKAPSGTPTKHPNWTNKQDNRDLESAQSLNRMEYIV

SEQ ID NO: 4 - protein, 264 aa, Rattus rattus
SGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTIQPDSIIEKASHSGMINPSRQWQTLKQNTGIAHFEYQIRVTCDDHYYGFGCNKFCRPRDDFFGHYACDQNGNKTCMEGWMGPECNKAICRQGCSPKHGSCKLPGDCRCQYGWQGLYCDKCIPHPGCVHGTCNEPWQCLCETNWGGQLCDK

SEQ ID NO: 5 - protein, 317 aa, Homo sapiens
YFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCE

SEQ ID NO: 6 - protein, 305 aa, Homo sapiens
SGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCE

SEQ ID NO: 7 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 8 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 9 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPVAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 10 - protein, 1999 aa, Homo sapiens
MQPPSLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPGCEVNPDNCVSHQCQNGGTCQDGLDTYTCLCPETWTGWDCSEDVDECEAQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 11 - protein, 1218 aa, Homo sapiens [sequence omitted]

SEQ ID NO: 12 - protein, 1238 aa, Homo sapiens
MRAQGRGRLPRRLLLLLALWVQAARPMGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTGLLVPVLCGAFSVLWLACVVLCVWWTRKRRKERERSRLPREESANNQWAPLNPIRNPIERPGGHKDVLYQCKNFTPPPRRADEALPGPAGHAAVREDEEDEDLGRGEEDSLEAEKFLSHKFTKDPGRSPGRPAHWASGPKVDNRAVRSINEARYAGKE

SEQ ID NO: 13 - protein, 231 aa, Homo sapiens
DVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVV

SEQ ID NO: 14 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 15 - protein, 587 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 16 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 17 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV